SEQUENCE LISTING (160 sequences)

SEQ ID NO: 1 - DNA, 1037 bp, Zea mays
ccaagggctaccccgtgccggttgccaaggaggagaggccggcgccgggcggcgacccgtgcccgcggtgcgggtcgcgggacaccaagttctgctactacaacaactacaacacgtcgcagccgcgccacttgtgcaagtcgtgccgccgctactggaccaagggcgggtccctccgcaacgtccccgtcgggggcggcacccgcaagagcagcagcagctcctcatcatcctcggcggcgacgacgacgacgacgacgacgtcaacctcgcccggcgccgcgcccaaggccacgaaacggtcgaagaactctaagcgccgccgcgtcgcgcctgccccggaccccgccgcgcccggcacggacgcctccaccgccgacgtcgcgagcacggcaccgtccgcagcgacggtggctgcctcggagaagccgagcgcgacggagcacgcagcggcggctgtcgccactgagaagccgccggccgcgcctccggtgtccgtcggcgctttcgcggacacgtccccggcgccagatgctgggagcggcggcgttagggagctgctgccgcacccgagccgcttcgagtggccgtcgggctgcgacctggggccgccctactggggctggggcaccagcgtgctcgccgacaccgacccggcgctgttcctcaatctgccgtgagtgacccgtgacaacgccatggcgcccatggttaactagtcaaccatcatcacacggtcgacgagatgagaggaacagagcacgcagtgcgtcgggctcttgaagctaggtgctgtagtagtctcctaagcttactgttgtctagaccgacagtctcgcgtacttgtgtagttgtgttgatggtttttaatcgccgctttgtactgcaattctagtaaaaaggctagcatgggatagccgggctctgcacggatctgtgcttgctttgacagtttgatctgattggggtgctttcttattttaaggccatgagaatcgcaagaacagggagcggcgaacttcgaagaaaaaaaaaaa

SEQ ID NO: 2 - DNA, 678 bp, Zea mays
aagggctaccccgtgccggttgccaaggaggagaggccggcgccgggcggcgacccgtgcccgcggtgcgggtcgcgggacaccaagttctgctactacaacaactacaacacgtcgcagccgcgccacttgtgcaagtcgtgccgccgctactggaccaagggcgggtccctccgcaacgtccccgtcgggggcggcacccgcaagagcagcagcagctcctcatcatcctcggcggcgacgacgacgacgacgacgacgtcaacctcgcccggcgccgcgcccaaggccacgaaacggtcgaagaactctaagcgccgccgcgtcgcgcctgccccggaccccgccgcgcccggcacggacgcctccaccgccgacgtcgcgagcacggcaccgtccgcagcgacggtggctgcctcggagaagccgagcgcgacggagcacgcagcggcggctgtcgccactgagaagccgccggccgcgcctccggtgtccgtcggcgctttcgcggacacgtccccggcgccagatgctgggagcggcggcgttagggagctgctgccgcacccgagccgcttcgagtggccgtcgggctgcgacctggggccgccctactggggctggggcaccagcgtgctcgccgacaccgacccggcgctgttcctcaatctgccgtga

SEQ ID NO: 3 - protein, 225 aa, Zea mays
KGYPVPVAKEERPAPGGDPCPRCGSRDTKFCYYNNYNTSQPRHLCKSCRRYWTKGGSLRNVPVGGGTRKSSSSSSSSSAATTTTTTTSTSPGAAPKATKRSKNSKRRRVAPAPDPAAPGTDASTADVASTAPSAATVAASEKPSATEHAAAAVATEKPPAAPPVSVGAFADTSPAPDAGSGGVRELLPHPSRFEWPSGCDLGPPYWGWGTSVLADTDPALFLNLP

SEQ ID NO: 4 - DNA, 1582 bp, Zea mays
cttgctatcctgatccaacgtcacactgatactatatactcatcgactgctgctagtatatactagctgcgcagtttgaacgacgaattgaaatcttcagctttttatttattattgattgactcgatcgtatatcttcctgttccacccatctcctatctattgatgctttattcatttccttttctcatctttaccctggttcgattttgcagcaccagcagcagcagcaccaccaccagcagcaaggccacggccacctgcagattgccgtcagcggcagcggcgacgccaatcatgagctcctccagcaaccaatcatggggggagcgcttcccgatggaggaggaggaggtggtggcgggggacaggttgggggccccgccaagcccatgtccatggcggagcgcgcgcgcctcgcgaggatcccactgccggagccgggactcaagtgcccgcgctgcgactcaaccaacaccaagttctgctacttcaacaactactccctcacgcagccgcgccacttctgccgggcctgccgccgctactggacgcgtggcggcgcgctccgcaacgtgccggtcggtggcggataccgccgccacgccaagcgcgccaagcccaagcagcagcagcagcacgcggccgccgggaccggagctgccaacggcgcgatgcagcagcctcccgctgggtctatggcgtcgtcggccgccgcctgcaccgcgaccacgacgatgacgaccaacgcgctggacgccgggcccggcggcatgctgcccatgctgccgccgctcgtccgcctagcagacttcgacgcaatgagcctcggctccagcttctccgggatatcgtccatggggaagcccggatccatcggcgcggcctgctacccgcactctgtcggcgggctggagcagtggagggtgcagcagatgcagagcttcccgttcttgcatgcgatggaccagggcccgctggggccacctctggccatggcgatggcggcgccaggagggatgttccagctaggtctagacaccaccagtgataacagccgtggcggtggcggcggcggctgcggcgaagacgggtcgtctgcgggagaggcgctccatatgatgcaagcagccaccaagagggagagctacccggcaccaccaagagccatgtacggcgaccaacaccacaatcacctcgctgctgctggtggctacacttcctattccaccaatgctgctgcaggtaaccatctcttgtaatggccggccgatcgatcgatcgagagctcaacaattcaagtgtcctgctatagctagctactacgacgtcgtgctacgatcgtatcggtttcggttcgttcctacaaatatagcctagagatagagtgtctctgtctgtgtgatcgatggtattgttatgatcatatagaaaagaccagtgtagcatgcatgcacttgttgcaatgtttgctttcaagaagaaactggagggaggagaggctcggtttggatgctgatcatgcacaaatattactagtgtctacagctgctacttcattataaaaaaaaaaaaaaaaaaa

SEQ ID NO: 5 - DNA, 870 bp, Zea mays
atgtccatggcggagcgcgcgcgcctcgcgaggatcccactgccggagccgggactcaagtgcccgcgctgcgactcaaccaacaccaagttctgctacttcaacaactactccctcacgcagccgcgccacttctgccgggcctgccgccgctactggacgcgtggcggcgcgctccgcaacgtgccggtcggtggcggataccgccgccacgccaagcgcgccaagcccaagcagcagcagcagcacgcggccgccgggaccggagctgccaacggcgcgatgcagcagcctcccgctgggtctatggcgtcgtcggccgccgcctgcaccgcgaccacgacgatgacgaccaacgcgctggacgccgggcccggcggcatgctgcccatgctgccgccgctcgtccgcctagcagacttcgacgcaatgagcctcggctccagcttctccgggatatcgtccatggggaagcccggatccatcggcgcggcctgctacccgcactctgtcggcgggctggagcagtggagggtgcagcagatgcagagcttcccgttcttgcatgcgatggaccagggcccgctggggccacctctggccatggcgatggcggcgccaggagggatgttccagctaggtctagacaccaccagtgataacagccgtggcggtggcggcggcggctgcggcgaagacgggtcgtctgcgggagaggcgctccatatgatgcaagcagccaccaagagggagagctacccggcaccaccaagagccatgtacggcgaccaacaccacaatcacctcgctgctgctggtggctacacttcctattccaccaatgctgctgcaggtaaccatctcttgtaa

SEQ ID NO: 6 - protein, 289 aa, Zea mays
MSMAERARLARIPLPEPGLKCPRCDSTNTKFCYFNNYSLTQPRHFCRACRRYWTRGGALRNVPVGGGYRRHAKRAKPKQQQQHAAAGTGAANGAMQQPPAGSMASSAAACTATTTMTTNALDAGPGGMLPMLPPLVRLADFDAMSLGSSFSGISSMGKPGSIGAACYPHSVGGLEQWRVQQMQSFPFLHAMDQGPLGPPLAMAMAAPGGMFQLGLDTTSDNSRGGGGGGCGEDGSSAGEALHMMQAATKRESYPAPPRAMYGDQHHNHLAAAGGYTSYSTNAAAGNHLL

SEQ ID NO: 7 - DNA, 1225 bp, Zea mays
gcccccctcctttcaactcgctcgctcgctcgccttccatctttctccctctgtcggcagtctgcagcagcccagcgccgtcgcgcatgcaggaggcgtcatcggcggcggcggcgggggccgagcccggccgtcgggcggcgcagcatcagttcgccggcgtggacctccggcggcccaaggggtacgcggcgccggcgccggcgccggcggtgggcgagggggacccgtgcccgcggtgtgcgtcgcgggacaccaagttctgctactacaacaactacaacacctcccagccgcgccacttctgcaagggctgccgccgctactggaccaagggtggcacgctgcgcaacgtccccgtcggcggcggcacccgcaagaagccctcctcctcctcctcgtcgtcgtcctacgtggccgccgcggacgccgacaggcagcccaagaagaagcccgccagcaagaagcgccgcgtcgtggcgccggccccggagctcgccaccgcggccgacccaggcaagacggcgaccaccaccacgacgacgagcgagatcaccacggagactggcgcgctggaggactccgactccctggcgcacctgctgctgcagcccgggacagaggacgcggaggccgtcgcgctcggcctcggcctctccgacttcccctccgccgggaaggcggtgctggacgacgaggactcgttcgtgtggcccgccgcgtcgttcgacatgggcgcgtgctgggccggcgcagggttcgccgacccggaccccgcctgcatcttcctcaacctcccgtgacagccacacacggtcacggcgccgaggcgtacgtcctcctgctctgctctgctctgcgcctgatgatgagtgcaaggaaatggagcgctttgattcttcagctatttgatgcatctgctaattgatttggcagtggtggtaaggtacacgcacgatgagctgactgcgtggttcttggtttgggccttagtatatgatgacgatgacgatgatactagttgtatgtgtgacgtgagagacgatcgcgacgacgaccttgaattttagtttctgagatgaaaaaggtcaactagtggcctctgtatgactttagttttactcatgaactcatcagatggctggctatggctgtggctgtggctgtggctaactcatcgtcaaaaaaaaaaaaaaaaaaaaaaaaaaaaaaaaaaaaaaaaaaa

SEQ ID NO: 8 - DNA, 723 bp, Zea mays
atgcaggaggcgtcatcggcggcggcgggggccgagcccggccgtcgggcggcgcagcatcagttcgccggcgtggacctccggcggcccaaggggtacgcgccgccggcgccggcggtgagcgagggggacccgtgcccgcggtgtgcgtcgcgggacaccaagttctgctactacaacaactacaacacctcccagccgcgccacttctgcaagggctgccgccgctactggaccaagggcggcacgctgcgcaacgtccccgtcggcggcggcacccgcaagaagccctccttgtcgtcgtcgtcgtcgtcgtcctacgcggccgccgcggacgccgacaggcagcccaagaagaagcccgccagcaagaagcgccgcgtcgtggcgccggccccggagctcgccgccgcggccgacccaggcaagacggcacccaccacgacgacgacgacgacgacgacgagcgagatcaccacggagactggcgcgctggaggactccgactccctggcgcacctgctgctgcagcccgggacagaggacgcggaggccgtcgcgctcgggctcggcctctccgacttcccctccgccgggaaggcggtgctggacgacgaggactcgttcgtgtggcccgccgcgtcgttcgacatgggcgcgtgctgggccggcgcagggttcgccgacccggaccccgcatgcatcttcctcaacctcccgtga

SEQ ID NO: 9 - protein, 240 aa, Zea mays
MQEASSAAAGAEPGRRAAQHQFAGVDLRRPKGYAPPAPAVSEGDPCPRCASRDTKFCYYNNYNTSQPRHFCKGCRRYWTKGGTLRNVPVGGGTRKKPSLSSSSSSSYAAAADADRQPKKKPASKKRRVVAPAPELAAAADPGKTAPTTTTTTTTTSEITTETGALEDSDSLAHLLLQPGTEDAEAVALGLGLSDFPSAGKAVLDDEDSFVWPAASFDMGACWAGAGFADPDPACIFLNLP

SEQ ID NO: 10 - DNA, 1490 bp, Zea mays
cttcttcccagcgacaagagaaaggattagaaaaaggaaagatccatggacatgatctccggcagcactgcagcaacatcaacaccccacaacaaccaacaggcggtgatgttgtcatcccccattataaaggaggaagctagggacccaaagcagacacgagccatgccccaaataggtggcagtggggagcgtaagccgaggccgcaactacctgaggcgctcaagtgcccacgctgcgactccaacaacaccaagttttgctactacaacaattatagcatgtcacaaccacgctacttttgcaaggcttgccgccgctattggacacatggtggtaccctccgcaatgtccccattggtggtgggtgtcgcaagaacaaacatgcctctagatttgtcttgggctctcacacctcatcgtcctcatctgctacctatgcaccattatcccctagcaccaacgctagctctagcaatatgagcatcaacaaacatatgatgatggtgcctaacatgacgatgcctaccccaacgacaatgggcttattccctaatgtgctcccaacacttatgccgacaggtggaggcgggggctttgacttcactatggacaaccaacatagatcattgtccttcacaccaatgtctctacctagccaggggccagtgcctatgctggctgcaggagggagtgaggcaacaccgtctttcctagagatgctgagaggagggatttttcatggtagtagtagctataacacaagtctcacgatgagtggtggcaacaatggaatggacaagccattttcgctgccatcatatggtgcaatgtgcacaaatgggttgagtggctcaaccactaatgatgccagacaactggtggggcctcagcaggataacaaggccatcatgaagagcagtaataacaacaatggtgtatcattgttgaacctctactggaacaagcacaacaacaacaacaacaacaacaacaacaacaacaacaacaacaacaacaagggacaataaggttagtgtgccagaccgtggaagcgttgctgctataaataatgcaattgggtagtagtacccagtgaaatcaggagagactagtagcctagggtgcattttgatttatttagttttggtcaagatgacaagtcatcatgaatcaccctttttattcatttgcatgttttgatgagagttcatttgcagtaagctatatatcattggataattaatcatttggttggatcatcagttcctatgggcattttcttcattttttctattctttaatttattaactaatgtatagcttttccaacttaactacttaatttggatagatgaagcatgttgtgtaacatgaattatggtagccagaaccgtatctatatactattggtaataacaatttgttgttgcatatgtatcttttgaatgtgttattccttttgtttcaactttcaatgagaaaattt

SEQ ID NO: 11 - DNA, 987 bp, Zea mays
atggacatgatctccggcagcactgcagcaacatcaacaccccacaacaaccaacaggcggtgatgttgtcatcccccattataaaggaggaagctagggacccaaagcagacacgagccatgccccaaataggtggcagtggggagcgtaagccgaggccgcaactacctgaggcgctcaagtgcccacgctgcgactccaacaacaccaagttttgctactacaacaattatagcatgtcacaaccacgctacttttgcaaggcttgccgccgctattggacacatggtggtaccctccgcaatgtccccattggtggtgggtgtcgcaagaacaaacatgcctctagatttgtcttgggctctcacacctcatcgtcctcatctgctacctatgcaccattatcccctagcaccaacgctagctctagcaatatgagcatcaacaaacatatgatgatggtgcctaacatgacgatgcctaccccaacgacaatgggcttattccctaatgtgctcccaacacttatgccgacaggtggaggcgggggctttgacttcactatggacaaccaacatagatcattgtccttcacaccaatgtctctacctagccaggggccagtgcctatgctggctgcaggagggagtgaggcaacaccgtctttcctagagatgctgagaggagggatttttcatggtagtagtagctataacacaagtctcacgatgagtggtggcaacaatggaatggacaagccattttcgctgccatcatatggtgcaatgtgcacaaatgggttgagtggctcaaccactaatgatgccagacaactggtggggcctcagcaggataacaaggccatcatgaagagcagtaataacaacaatggtgtatcattgttgaacctctactggaacaagcacaacaacaacaacaacaacaacaacaacaacaacaacaacaacaacaacaagggacaataa

SEQ ID NO: 12 - protein, 328 aa, Zea mays
MDMISGSTAATSTPHNNQQAVMLSSPIIKEEARDPKQTRAMPQIGGSGERKPRPQLPEALKCPRCDSNNTKFCYYNNYSMSQPRYFCKACRRYWTHGGTLRNVPIGGGCRKNKHASRFVLGSHTSSSSSATYAPLSPSTNASSSNMSINKHMMMVPNMTMPTPTTMGLFPNVLPTLMPTGGGGGFDFTMDNQHRSLSFTPMSLPSQGPVPMLAAGGSEATPSFLEMLRGGIFHGSSSYNTSLTMSGGNNGMDKPFSLPSYGAMCTNGLSGSTTNDARQLVGPQQDNKAIMKSSNNNNGVSLLNLYWNKHNNNNNNNNNNNNNNNNKGQ

SEQ ID NO: 13 - DNA, 1665 bp, Zea mays
cttttctctatatacaactaaaggactcatcatctctcttcctctccctcgtcttcctgcgtgcacaaccggatccttcgccttgctactgttcggcgcttccacaacagctagcttgctgaaaggaaggagccagcgcccatggacatgacctccaacaccagcaacagcgctgcagcaacatcatcggctccccacaaccaccagccggtgcaggaggccatggtgccatctccaaccggaaaggagcaaggcaggaacaccaagaaagcgcggccgggccggcggcaacggggaactaagccgcggccactacaggacacggcgctcaactgcccgcgctgctgctccaccaacaccaagttttgctactacaacaactacaacctgacgcagccgcgctacttctgcaaggcatgccgccgctactggacacaaggcggcaccctccgcaacgtccccgtcggcggcggctgccgcaggaacaagcgcgcctccggctcctgctcgtcctctgccgccgccgccgcaccgctatgctccagcgctaccgccaccggcgccgagatggtgaacaccgtcaacacgcgcctgatgctgatgtctaccgccggtaccaccatgtcgacgacgatatccatgccctccccggcgacaggcttgtttccgagtagcatgctcccgacgacatttacgccgacaggcagtagcggcttccacctcgccatggacgaccagcagcagcagggtttcctgcccttcgcgccacagtctttgtcccaccaccatcaccaggcgctggagctggctcctgcagggaatgacacgacgccaactttcctggacatgctgacaggagggtatcttgatggcggtagaagcatcagcgacagcagctacggcgctggtctcgccatgagcggtggtggcagcaatggaatggacatgccgtttctgctgcctgggatgggggccgcctcagcaaccgatcccatgcagacgcagctgctggcgggaatgagaatgaacaatgtagcaggtgggctccagtgggtgtcatcagagcctgacaactacaccaacgacgacggtgcttatgcagcaggaccagccgctggactgcagcatcaccagcagcagcaggttgttggcgatcagcagcagcagcaggagaacaaagcgaatcagcgcagcaaaaacaacaacggcggcggcggcggtgggtcgtcggtgtacagcttctactggaccaacaccagcaacagcgatggggcagagttgtagtgtgcctctgccagtgctagagccacaggagacgcgaaatcgctgctgcaaactccctcgcgacgaggaaggggacgcgattcaccgttaagcactagctcagtttatagaggataggatgcatattcgcgtttgattaatctgcattgtggtcgtgatggcaagtcatcatgcacatcgccctttttgttcaaatgcatgcgttagttcgttttcatcagagtttacttgctgcagtctatcatcgccggcagagattagttaggtgtctatgcttttatattcatcagtagttctggataataatgtcttcgtattcctcagtttctttgttaagtcgtctaaaactttccatgtgtaaaaaaaaaaaaaaaaaaaaaa

SEQ ID NO: 14 - DNA, 1143 bp, Zea mays
atggacatgacctccaacaccagcaacagcgctgcagcaacatcatcggctccccacaaccaccagccggtgcaggaggccatggtgccatctccaaccggaaaggagcaaggcaggaacaccaagaaagcgcggccgggccggcggcaacggggaactaagccgcggccactacaggacacggcgctcaactgcccgcgctgctgctccaccaacaccaagttttgctactacaacaactacaacctgacgcagccgcgctacttctgcaaggcatgccgccgctactggacacaaggcggcaccctccgcaacgtccccgtcggcggcggctgccgcaggaacaagcgcgcctccggctcctgctcgtcctctgccgccgccgccgcaccgctatgctccagcgctaccgccaccggcgccgagatggtgaacaccgtcaacacgcgcctgatgctgatgtctaccgccggtaccaccatgtcgacgacgatatccatgccctccccggcgacaggcttgtttccgagtagcatgctcccgacgacatttacgccgacaggcagtagcggcttccacctcgccatggacgaccagcagcagcagggtttcctgcccttcgcgccacagtctttgtcccaccaccatcaccaggcgctggagctggctcctgcagggaatgacacgacgccaactttcctggacatgctgacaggagggtatcttgatggcggtagaagcatcagcgacagcagctacggcgctggtctcgccatgagcggtggtggcagcaatggaatggacatgccgtttctgctgcctgggatgggggccgcctcagcaaccgatcccatgcagacgcagctgctggcgggaatgagaatgaacaatgtagcaggtgggctccagtgggtgtcatcagagcctgacaactacaccaacgacgacggtgcttatgcagcaggaccagccgctggactgcagcatcaccagcagcagcaggttgttggcgatcagcagcagcagcaggagaacaaagcgaatcagcgcagcaaaaacaacaacggcggcggcggcggtgggtcgtcggtgtacagcttctactggaccaacaccagcaacagcgatggggcagagttgtag

SEQ ID NO: 15 - protein, 380 aa, Zea mays
MDMTSNTSNSAAATSSAPHNHQPVQEAMVPSPTGKEQGRNTKKARPGRRQRGTKPRPLQDTALNCPRCCSTNTKFCYYNNYNLTQPRYFCKACRRYWTQGGTLRNVPVGGGCRRNKRASGSCSSSAAAAAPLCSSATATGAEMVNTVNTRLMLMSTAGTTMSTTISMPSPATGLFPSSMLPTTFTPTGSSGFHLAMDDQQQQGFLPFAPQSLSHHHHQALELAPAGNDTTPTFLDMLTGGYLDGGRSISDSSYGAGLAMSGGGSNGMDMPFLLPGMGAASATDPMQTQLLAGMRMNNVAGGLQWVSSEPDNYTNDDGAYAAGPAAGLQHHQQQQVVGDQQQQQENKANQRSKNNNGGGGGGSSVYSFYWTNTSNSDGAEL

SEQ ID NO: 16 - DNA, 1553 bp, Zea mays
cccgtgtaccgtacacagcaggatcctccgccttccttgtgttccgcgcttccacaacagtttaccgcaaggaaggaaccatggacatgaactccaacgccaacaacagcactgccgcagcagcatcggctcccatcaacaaccagcaggaggctgtggtgtcatccccaaccagaaaggagcaagccaggaaccccaagaaggcgcgggcggcgccgcagcaggcgggcggcagcggggagcctaggccgcggcctccgccggacgcggcgcacagctgcccgcgctgctcctccaccaacacaaagttctgctactacaacaactacaacctgacgcagccgcgctacttctgcaagacgtgccgccgctactggacacacggcggcaccctccgcaacgtccccgtcggcggcggctgccgcaggaacaagcgcgcctccagctcctcgtccccgtttccgggcccctccagcaccgccgccaccagcgccgcgatggagaagaccgtcagcacgcggctgatgctgatggcgaccagcaccatggcgatgccctccccgacggcaggcctgtttgttcccgatgacatgtccccggcattcacgccgacgacgggcggtagcggcttcgacgacctcgccggcatggacgagcagcaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgacgacggtggttatgcagcaggaccagccgccggagtgcagcagcagcagcagcagcagcagcagcagattaatggtggtgatcaccagaagcaggacgagaacaaagaggcggggaacggcaaaggcaacgacgacggcggcggcgggtcgtcgtcggtgtacagcttctggatgaacaccagcggcagcgacggggcagaggggtagtgcgccactgccagtgccagccacaggaggaggcgaaagtgctgctgcaaactccctcgcatcatctggtcggtgcaagtgcagcagacatccttcgtcgacgcaatcaaatcatcaaaaggtggcaacagggagtgaaagggggaagaagttcaccatccagcgtagaggatagggtgcatgttcatgtttgatttatctgcattgtggtcggtcgtggttgcaagtcatcttgcaccaccctttgttttggttcagtttgcatgcgttcgttcgtcagagttttacttgcagcagtctttggatcgccggcagagattagttaggtatctatgctttgttttcgtcagttcgttctggataatgtcttaataattcctcagtttatttctttgttaactaaaaaaaaaaaaaaaa

SEQ ID NO: 17 - DNA, 1056 bp, Zea mays
atggacatgaactccaacgccaacaacagcactgccgcagcagcatcggctcccatcaacaaccagcaggaggctgtggtgtcatccccaaccagaaaggagcaagccaggaaccccaagaaggcgcgggcggcgccgcagcaggcgggcggcagcggggagcctaggccgcggcctccgccggacgcggcgcacagctgcccgcgctgctcctccaccaacacaaagttctgctactacaacaactacaacctgacgcagccgcgctacttctgcaagacgtgccgccgctactggacacacggcggcaccctccgcaacgtccccgtcggcggcggctgccgcaggaacaagcgcgcctccagctcctcgtccccgtttccgggcccctccagcaccgccgccaccagcgccgcgatggagaagaccgtcagcacgcggctgatgctgatggcgaccagcaccatggcgatgccctccccgacggcaggcctgtttgttcccgatgacatgtccccggcattcacgccgacgacgggcggtagcggcttcgacgacctcgccggcatggacgagcagcaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgacgacggtggttatgcagcaggaccagccgccggagtgcagcagcagcagcagcagcagcagcagcagattaatggtggtgatcaccagaagcaggacgagaacaaagaggcggggaacggcaaaggcaacgacgacggcggcggcgggtcgtcgtcggtgtacagcttctggatgaacaccagcggcagcgacggggcagaggggtag

SEQ ID NO: 18 - protein, 351 aa, Zea mays
MDMNSNANNSTAAAASAPINNQQEAVVSSPTRKEQARNPKKARAAPQQAGGSGEPRPRPPPDAAHSCPRCSSTNTKFCYYNNYNLTQPRYFCKTCRRYWTHGGTLRNVPVGGGCRRNKRASSSSSPFPGPSSTAATSAAMEKTVSTRLMLMATSTMAMPSPTAGLFVPDDMSPAFTPTTGGSGFDDLAGMDEQHQQGFLPFSPLSLSDQAPELAPGGGGDTTPSFLDMLTGGYLDGGGYGGMSGGSDAMDMPFSLPEMGPPTTDPMPFQLQWTSSELDNYINDDGGYAAGPAAGVQQQQQQQQQQINGGDHQKQDENKEAGNGKGNDDGGGGSSSVYSFWMNTSGSDGAEG

SEQ ID NO: 19 - DNA, 1553 bp, Zea mays; identical to SEQ ID NO: 16

SEQ ID NO: 20 - DNA, 1056 bp, Zea mays; identical to SEQ ID NO: 17

SEQ ID NO: 21 - protein, 351 aa, Zea mays; identical to SEQ ID NO: 18

SEQ ID NO: 22 - DNA, 1585 bp, Zea mays
tttgttttctagcttttgtcagcgaaccttcgccctgaaaaccccacccactcttcctctccgatcctcctagcgcctcacttgcttcattccattccattccattccactacccaatacccatctccccgccggcctttcccctcccgcgtgccgatctgcctccggaggctagctcgaccggccgatctagctcgcgcgccgccgctttcagctcgatcagctgccgcacgtcgtccaccgacccgccgtcctgtgagcgcgatcgacagattactaagatggctggcgcggggtgtgcgacagccgtgcagcggccggcggcggcgggggcgccggctgctgtggcaaggaccgcggtcggggcaggcggcgcggtcgccgacccgcgcgcggaggcgctgcggtgcccgcgctgcgactcggccaacaccaagttctgctactacaacaactactcgctgtcgcagccgcgccacttctgcaaggcgtgcaagcgctactggacgcgcgggggcacgctccgcaacgtccccgtcggcgggggctgccgcaagaacaagcgctcctccaggagcggcggcggcgggaggaatgggtcctcctcctccttctcctcctcctcggcggactcgacggcgctgcccctcccgccgcctccgacgacgatggggtccctgccgtcgtcgctggggctgcccgggggcgcgtcgctcctcctcgggtccgcgctccctggcggtgccggtgaccaccaccacctcggccttttccaggcggccatgcagtcggtggtctcctccgacgcgaccgcctacgacgagatgcagcagcaacagcagacgcagctggaccacctgctgggcctcggatacggaggaggcgccggcgcgcagatccagctcaagccgtggatgcaggaggccgccggggcaggcggcgcgcccgggatcatggacagcttctacgcgccgctgctgtccagctccctcgtgccggggctggaggagctgcacgtcaaggcggaggccgccggagccggggatcaccagcagaagccgtcatcaagggaccagcagagcgccagctgggactgggagctgccgacgccgtcgtcgtccaacgtcgacgccaacgtcgtcaccgcgtctgacgcgctcatggccgccgccgccgccgcgtccatgaaccccgctgttagtgttagctccacgccctccacggcaccaaccgccccctcctcgttcttatactggggcaacagcggcattggcggcgctgccgcggcctggccagacctcgccaactgcggatcctccattgccacgctcttctagccaagccgcgcctggctgtcctgctggcctcgttaacttggtttttttgtaccttaatttctggttaatttttttcttgactgaaataacatgtaaatttgcggggaaaagaaaactgagatgagagagagagaacgctgtgaaagaagagagctagaccatctcaccacctcatagggcagaaacttagacaaatcaaacaaatgatccaatccaatatttcaagagaattccttcgccgcgactat

SEQ ID NO: 23 - DNA, 1056 bp, Zea mays
atggctggcgcggggtgtgcgacagccgtgcagcggccggcggcggcgggggcgccggctgctgtggcaaggaccgcggtcggggcaggcggcgcggtcgccgacccgcgcgcggaggcgctgcggtgcccgcgctgcgactcggccaacaccaagttctgctactacaacaactactcgctgtcgcagccgcgccacttctgcaaggcgtgcaagcgctactggacgcgcgggggcacgctccgcaacgtccccgtcggcgggggctgccgcaagaacaagcgctcctccaggagcggcggcggcgggaggaatgggtcctcctcctccttctcctcctcctcggcggactcgacggcgctgcccctcccgccgcctccgacgacgatggggtccctgccgtcgtcgctggggctgcccgggggcgcgtcgctcctcctcgggtccgcgctccctggcggtgccggtgaccaccaccacctcggccttttccaggcggccatgcagtcggtggtctcctccgacgcgaccgcctacgacgagatgcagcagcaacagcagacgcagctggaccacctgctgggcctcggatacggaggaggcgccggcgcgcagatccagctcaagccgtggatgcaggaggccgccggggcaggcggcgcgcccgggatcatggacagcttctacgcgccgctgctgtccagctccctcgtgccggggctggaggagctgcacgtcaaggcggaggccgccggagccggggatcaccagcagaagccgtcatcaagggaccagcagagcgccagctgggactgggagctgccgacgccgtcgtcgtccaacgtcgacgccaacgtcgtcaccgcgtctgacgcgctcatggccgccgccgccgccgcgtccatgaaccccgctgttagtgttagctccacgccctccacggcaccaaccgccccctcctcgttcttatactggggcaacagcggcattggcggcgctgccgcggcctggccagacctcgccaactgcggatcctccattgccacgctcttctag

SEQ ID NO: 24 - protein, 351 aa, Zea mays
MAGAGCATAVQRPAAAGAPAAVARTAVGAGGAVADPRAEALRCPRCDSANTKFCYYNNYSLSQPRHFCKACKRYWTRGGTLRNVPVGGGCRKNKRSSRSGGGGRNGSSSSFSSSSADSTALPLPPPPTTMGSLPSSLGLPGGASLLLGSALPGGAGDHHHLGLFQAAMQSVVSSDATAYDEMQQQQQTQLDHLLGLGYGGGAGAQIQLKPWMQEAAGAGGAPGIMDSFYAPLLSSSLVPGLEELHVKAEAAGAGDHQQKPSSRDQQSASWDWELPTPSSSNVDANVVTASDALMAAAAAASMNPAVSVSSTPSTAPTAPSSFLYWGNSGIGGAAAAWPDLANCGSSIATLF

SEQ ID NO: 25 - DNA, 1638 bp, Zea mays
attctccaccacctccgagcttgccagaggactccacctctttcttctcgcatgcttccttttctttccaaacacaaatgcgcagcaaagaaagtgcaaatcataaagaaaagagcagaattcaaagcagccggaaagctgactagctagaaggagtgaagagagagaagtatatctcagcaacgcctccatggtcttcccttcaccttcagtgcaggtctacctagatccacatccacctaattggaataaccagcagcagcaaggtcagcagccgagggcgaatgggggagctgatgcaccgttgctgcccctgggtccggcggcagctacatcggcggctccggagcaaggtgggttgcctagaagctcatcaatggctaatgccgccgtggctgcgcacgcgaggcccaactcaatggcggagcgcgcgcggctggcgcggatgccgcatccggagccagcgctcaagtgcccgcgttgcgaatccaccaacaccaagttctgctactacaacaactactccctctcccagccccgccacttctgcaagacgtgccgccgctactggacgcgcggcggatcccttcgcaacgtccccgtaggtggaggctgccgtcgcaacaagcgttcgtccaagtcctcatcctcctctgctgggtcttcctcctcaaagatgtctccctcaggaaggctactgggtggtccatcagctacaccgtccaccaccccaggcactaccggtgcgatcattactccgggtctcagctctttctctcaccacttgccgttcttgggctccatgcacccgtcagggcccaacctaggtctagctttctccgccggactgccgctagtcggcatgcagcacctggacacggtggatcagtttccggtggcaagcggcggcggcaccaccataggtgcatctctagagcagtggagagtgcgacagcagcagcagcagttcccattcatgactgggggaatactggatctgtcgcagccgccgacgtaccaattcggtttggaagctaaccgaggaggcagcggctcagctgcggtggcgttcaattcaggacagaccacgacgaccagtgctaccacgggaaggcaggaagggtcatcaaagaagatgggggatagtaaaggagaagatatgagcttacagaagcagtacatggtacctctacgccacggatcaggatcacacggtgtctgggatgggagtgctggtggaactggcagcaacggtggtggtactggcaatggcggttcaagttggccaatgaacatgattcctggattccattcttcgtccactagcggttgcaatgacagtggcttgtcgtagtactctctagctaggggctggggcaatgtcctatcaagaaaaatattttggagatgcatggttgagtctgagcaaatgctagctatagctaggacgatggaggaacaggcgatgaccaatccacaggcatgttcatcatctgctattcttcatcggaattgtagaagcacactgctacctcatgatggtcacaggtaggagtttaaagtaaagctgctctttgtttaaacctttttctgtcatttggttttgaacttaaaggtgttctctgcattgtgtccata

SEQ ID NO: 26 - DNA, 1158 bp, Zea mays
atggtcttcccttcaccttcagtgcaggtctacctagatccacatccacctaattggaataaccagcagcaaggtcagcagccgagggcgaatgggggagctgatgcaccgttgctgcccgtgggtccggcggcagctacatcggcggctccggagcaaggtgggttgcctagaagctcatcaatggctaatgccgccgtggctgcgcacgcgaggcccaactcaatggcggagcgcgcgcggctggcgcggatgccgcatccggagccagcgctcaagtgcccgcgttgcgaatccaccaacaccaagttctgctactacaacaactactccctctcccagccccgccacttctgcaagacgtgccgccgctactggacgcgcggcggatcccttcgcaacgtccccgtaggtggaggctgccgtcgcaacaagcgttcgtccaagtcctcatcctcctctgctgggtcttcctcctcaaagatgtctccctcaggaaggctactgggtggtccatcagctacaccgtccaccaccccaggcactaccggtgcgatcattactccgggtctcagctctttctctcaccacttgccgttcttgggctccatgcacccgtcagggcccaacctaggtctagctttctccgccggactgccgctagtcggcatgcagcacctggacacggtggatcagtttccggtggcaagcggcggcggcaccaccataggtgcatctctagagcagtggagagtgcgacagcagcagcagcagttcccattcatgactgggggaatactggatctgtcgcagccgccgacgtaccaattcggtttggaagctaaccgaggaggcagcggctcagctgcggtggcgttcaattcaggacagaccacgacgaccagtgctaccacgggaaggcaggaagggtcatcaaagaagatgggggatagtaaaggagaagatatgagcttacagaagcagtacatggtacctctacgccacggatcaggatcacacggtgtctgggatgggagtgctggtggaactggcagcaacggtggtggtactggcaatggcggttcaagttggccaatgaacatgattcctggattccattcttcgtccactagcggttgcaatgacagtggcttgtag

SEQ ID NO: 27 - protein, 385 aa, Zea mays
MVFPSPSVQVYLDPHPPNWNNQQQGQQPRANGGADAPLLPVGPAAATSAAPEQGGLPRSSSMANAAVAAHARPNSMAERARLARMPHPEPALKCPRCESTNTKFCYYNNYSLSQPRHFCKTCRRYWTRGGSLRNVPVGGGCRRNKRSSKSSSSSAGSSSSKMSPSGRLLGGPSATPSTTPGTTGAIITPGLSSFSHHLPFLGSMHPSGPNLGLAFSAGLPLVGMQHLDTVDQFPVASGGGTTIGASLEQWRVRQQQQQFPFMTGGILDLSQPPTYQFGLEANRGGSGSAAVAFNSGQTTTTSATTGRQEGSSKKMGDSKGEDMSLQKQYMVPLRHGSGSHGVWDGSAGGTGSNGGGTGNGGSSWPMNMIPGFHSSSTSGCNDSGL

SEQ ID NO: 28 - DNA, 1729 bp, Zea mays
gctcctctccctgctccgacgtctttataaaccgccacaccccggtaccactaccccaccccagcccccatcctctgattcataccacaaagctagaacctgaaagcagccagccacaccactccgctccactccacctttgccatctccaagaaagaacccagcctcttctgcttcaagaagccttttttgatcctgagttatcgccatccatcaccatgatccaagaactgctcggcggggccgccatggaccagctcaagagcgtgaatgagtccctgcccctgttgctgcactcggtcatctccaacccatcgcccacgtcgtcgtcgtcgacgtcgtcgcgctcgtctgcgcagcagcatcagcagcagcggtcgacgtcggcaacgtcgtcgccgcaagcagggcagcagcagcagcagcagggccaggggcaaggagcggagcagacgcctctgcggtgccccaggtgtaactcctccaacaccaagttctgctactacaacaactacaacctcacccagccgcgccacttctgcaagacgtgccgccggtactggaccaagggcggcgcgctccgcaacgtgcccatcggcggcggctgccgcaagccgcgccccatgcccacgcccgtcacgaagccggcggtctcatgcaaggccgtgggcggcgcgcagtcgctgggcctcggcgtcggcttgggcatgggcgccggccccggaccctgggcgtcctcgcagcaggcggccgccgcgcagctcatggcgctgctcaacagcgccaggagcgtgcagggaggcggcggcggcaacatgcacaggctcctcggcctcgacgccgtggcccacctgcccctccatgtcctgccgggcgcgggcaataatgccggtggcacggcgccgtcgttctggccgcaggccgcgccgcgtgtcatccccgcaccgccgcacatggactcccagctcggcatggggccgctgggccagcacgacgtgctgtcgagcctcgggctaaagctgcccccgccatcgccgtcgccggcggcaagctactacagcgaccagctgcacgcggtggtgagcagcgccgccggacgcggacacgagtacgaaacagccgcctgcgccacgtcactgccttgcaccacggcgctgacctccctcccgccggccgcgtcgagcgtgtccgctgcactggccagcgccgccacggtcgggctagacctcccgctggtctccctctccgcgcccgagatgcagtactgggccgggccggcggcgatgtccgtggcgtggccggacttgcccacccccaacggcgcgttcccgtgagagacaaacatggacctcatctgctgtacggatggcgcagctgtttacccacccacatgacacactagctagtacgtaatcagtagctcgatcacttcagccttcagtgtagctttatcttgccgttaatttgactgtgttatgagtgagaactccactctgggggaggttattaatcaatggtgtttctattactcttgtttttgttcactagtgggagcatgggagtaacttttcttactgttattaatcagagtttttattactcttgttggtaatatctttttctatttatcttctctatatctgcatgcttgtgagctgtatgtagtgtagtagcaatagtagaagtgatccataactttgtatctgttgttaaaaaaaaaaaaaaaaaa

SEQ ID NO: 29 - DNA, 1392 bp, Zea mays
atgatccaagaactgctcggcggggccgccatggaccagctcaagagcgtgaatgagtccctgcccctgttgctgcactcggtcatctccaacccatcgcccacgtcgtcgtcgtcgacgtcgtcgtcgcgctcgtctgcgcagcagcatcagcagcagcggtcgacgtcggcaacgtcgtcgccgcaagcagggcagcagcagcagcagcagggccaggggcaaggagcggagcagacgcctctgcggtgccccaggtgtaactcctccaacaccaagttctgctactacaacaactacaacctcacccagccgcgccacttctgcaagacgtgccgccggtactggaccaagggcggcgcgctccgcaacgtgcccatcggcggcggctgccgcaagccgcgccccatgcccacgcccgtcacgaagccggcggtctcatgcaaggccgtgggcggcgcgcagtcgctgggcctcggcgtcggcttgggcatgggcgccggccccggaccctgggcgtcctcgcagcaggcggccgccgcgcagctcatggcgctgctcaacagcgccaggagcgtgcagggaggcggcggcggcaacatgcacaggctcctcggcctcgacgccgtggcccacctgcccctccatgtcctgccgggcgcgggcaataatgccggtggcacggcgccgtcgttctggccgcaggccgcgccgcgtgtcatccccgcaccgccgcacatggactcccagctcggcatggggccgctgggccagcacgacgtgctgtcgagcctcgggctaaagctgcccccgccatcgccgtcgccggcggcaagctactacagcgaccagctgcacgcggtggtgagcagcgccgccggacgcggacacgagtacgaaacagccgcctgcgccacgtcactgccttgcaccacggcgctgacctccctcccagctcggcatggggccgctgggccagcacgacgtgctgtcgagcctcgggctaaagctgcccccgccatcgccgtcgccggcggcaagctactacagcgaccagctgcacgcggtggtgagcagcgccgccggacgcggacacgagtacgaaacagccgcctgcgccacgtcactgccttgcaccacggcgctgacctccctcccgccggccgcgtcgagcgtgtccgctgcactggccagcgccgccacggtcgggctagacctcccgctggtctccctctccgcgcccgagatgcagtactgggccgggccggcggcgatgtccgtggcgtggccggacttgcccacccccaacggcgcgttcccgtgagagacaaacatggacctcatctgctgtacggatggcgcagctgtttacccacccacatgacacattga

SEQ ID NO: 30 - protein, 463 aa, Zea mays
MIQELLGGAAMDQLKSVNESLPLLLHSVISNPSPTSSSSTSSSRSSAQQHQQQRSTSATSSPQAGQQQQQQGQGQGAEQTPLRCPRCNSSNTKFCYYNNYNLTQPRHFCKTCRRYWTKGGALRNVPIGGGCRKPRPMPTPVTKPAVSCKAVGGAQSLGLGVGLGMGAGPGPWASSQQAAAAQLMALLNSARSVQGGGGGNMHRLLGLDAVAHLPLHVLPGAGNNAGGTAPSFWPQAAPRVIPAPPHMDSQLGMGPLGQHDVLSSLGLKLPPPSPSPAASYYSDQLHAVVSSAAGRGHEYETAACATSLPCTTALTSLPARHGAAGPARRAVEPRAKAAPAIAVAGGKLLQRPAARGGEQRRRTRTRVRNSRLRHVTALHHGADLPPAGRVERVRCTGQRRHGRARPPAGLPLRARDAVLGRAGGDVRGVAGLAHPQRRVPVRDKHGPHLLYGWRSCLPTHMTH

SEQ ID NO: 31 - DNA, 1257 bp, Zea mays
cttcgatctccaatccccagctagctaactatccaatggcgggccaagtgatggaagctcaggcgtgccggctgcagcctcccaccatggcgccgttcgcgccactacctcatcagtacaacaacacctgcaagcacctcaacacgacgatggccgccacttccggcaacaacaacagcaacggcagcaccgctaccgcggctggcggtgcggcggccgacggcatggccgcctacctccagcacctgcagcaggcgggcgccgaggcggcggccaagagcggcggcggcggcggcgcggcgcgcggggagcagtgcccgcggtgcgcgtcgcgcgacaccaagttctgctactacaacaactacaacacgtcccagccgcgccacttctgccgcgcctgccgacgctactggacgctgggcgggtccctccgcaacgtccccgtcggcgggtccacgcgcaagcgcccgcgcctggcgcaccaccaccagcacgccaggctcgctcctccggcgccgcacgtcttcggcctcacgccgatgatgcctcctcccttgcaaccgtcgtcgtcgacatcgtcgtcgcagggacagggacagggacagggacagagcggcggcctcctcggctcactgttcgcgctcggcgccgcgggcgcggggccgccgctgctcgaaggccgcggcgcagggtcgtcgttcgacttcgacctcgggctcggactcccgacgggggccttacacctgggcgcggccggacaaatgcaaggcctcgggctcatggggggaggaggtgcctccgcagccgggtcgtcgtccttcctctggcccgccgcggggctgctggtggacaacgacagcgtggacacgtggaagatgccaggcgccgctgcgggttcaatgtggccagacttctcttttccggcggcagcggcgccacaaaccgccggattgcttcatggcggaggccacctgatgtgagacgagacgccgcgcttccttcgacgtactaccccctagcgctagccctagattctagcgcatgcagcccggtgtccgggccggtttgcattgcatagcatacatagtagttggtctttttttcagcattgtattattggatcatgtgtacgtaattaagccgtgcttcagttttccttgactctgttaagctataaagggccctccaaatctgcagttttggggcgtttgtttcagaaaatcatattctagaattcgattctatcacaaaaaaaaa

SEQ ID NO: 32 - DNA, 945 bp, Zea mays
atggcgggccaagtgatggaagctcaggcgtgccggctgcagcctcccaccatggcgccgttcgcgccactacctcatcagtacaacaacacctgcaagcacctcaacacgacgatggccgccacttccggcaacaacaacagcaacggcagcaccgctaccgcggctggcggtgcggcggccgacggcatggccgcctacctccagcacctgcagcaggcgggcgccgaggcggcggccaagagcggcggcggcggcggcgcggcgcgcggggagcagtgcccgcggtgcgcgtcgcgcgacaccaagttctgctactacaacaactacaacacgtcccagccgcgccacttctgccgcgcctgccgacgctactggacgctgggcgggtccctccgcaacgtccccgtcggcgggtccacgcgcaagcgcccgcgcctggcgcaccaccaccagcacgccaggctcgctcctccggcgccgcacgtcttcggcctcacgccgatgatgcctcctcccttgcaaccgtcgtcgtcgacatcgtcgtcgcagggacagggacagggacagggacagagcggcggcctcctcggctcactgttcgcgctcggcgccgcgggcgcggggccgccgctgctcgaaggccgcggcgcagggtcgtcgttcgacttcgacctcgggctcggactcccgacgggggccttacacctgggcgcggccggacaaatgcaaggcctcgggctcatggggggaggaggtgcctccgcagccgggtcgtcgtccttcctctggcccgccgcggggctgctggtggacaacgacagcgtggacacgtggaagatgccaggcgccgctgcgggttcaatgtggccagacttctcttttccggcggcagcggcgccacaaaccgccggattgcttcatggcggaggccacctgatgtga

SEQ ID NO: 33 - protein, 314 aa, Zea mays
MAGQVMEAQACRLQPPTMAPFAPLPHQYNNTCKHLNTTMAATSGNNNSNGSTATAAGGAAADGMAAYLQHLQQAGAEAAAKSGGGGGAARGEQCPRCASRDTKFCYYNNYNTSQPRHFCRACRRYWTLGGSLRNVPVGGSTRKRPRLAHHHQHARLAPPAPHVFGLTPMMPPPLQPSSSTSSSQGQGQGQGQSGGLLGSLFALGAAGAGPPLLEGRGAGSSFDFDLGLGLPTGALHLGAAGQMQGLGLMGGGGASAAGSSSFLWPAAGLLVDNDSVDTWKMPGAAAGSMWPDFSFPAAAAPQTAGLLHGGGHLM

SEQ ID NO: 34 - DNA, 1494 bp, Zea mays
ggccgagcgggtggagctctagccacgcccacgctcccgcctccccggcaaacacactcacgcccgccgaggccacatcctaacacgttgtagacgccgccaccgcctgacgccatgcaggagttccagtccatccctggcctggccgggcggctcttcggcggggccgcggcccccggcgacctccggcgcgcgcaggcggcgcagcatggccccggcggagcgcggtgcggcggtgcctcgcccgcggcgccggaggcggtgaagtgcccgcggtgcgagtctaccaacaccaagttctgctactacaacaactacaacctgtcgcagccgcgccacttctgcaagagctgccgccggtactggaccaagggcggcgtcctgcgcaacgtccccgtgggcggcggctgccgcaaggcgaagcgcgcctcgtcctcggcgtcggcgtcggcgtcggtgtcctcgcccgcctcgtcctcggcgccgtccacgcccgcgtcggccgacgcgggcaagaacccgcgccgcgcctcggcgtcctcgccctcgccccgctccaacagcggcagcgcgagccccacggccgccgccgcgacgaccccgaccccgaccgatccgaccacccccgccacgccgtcgtcgaacggcgtcgccttcacgggcagccaccactcgtcgaaccccttctccacgatcgacgtggcggccgcgccaccggcgccgatattcgccgaccaggcggcggcgctggcgtccctcttcgcgcctccccctccgccgccgctcccggtgttcagcttcgcggcggcgcagccgaaggaggaggaagcgcccaccaattcggagctgcagcaacacctcgccgcacaggcggcgccgtcgtcgtcggtctccgaggacatcatggcgccgttcgcatccctggacgccgctgggatattcgagctcggcgacgccgcgtcagccgcggcgtactggagcgccgggagctgctgggcggacgtccaggacccgagcttgtacctaccctagcgtacgtgcttgtttagttacctatcgcgatcgcttgattaaagccgcggttagatcttaacgacggccggccacgagctccgtctctctagctattactccatgtcctccctgcctctctctctctgtcttgtttattggtgatttggcttcacaggctgcgtgtctagaaagctaacatctgtgttccgtgtctgcattcaccctgcgagtgtggcggatcattgcgctgctgcgatagaattaatcgcattattattgcttgtttactctttgtaatagtaacttgtatgaatacatgcatgtttggggggggggggaagcaacgtaacgtaaccacgagtgtccgtgtgttatgttgtgatgcgcggtcaaagccgatggtcaggtgtatcgccaggggatggatgatggaacaatggtgcgtgtcattttggttaaaaaaaaaaaaaaaaaaaaaaa

SEQ ID NO: 35 - DNA, 918 bp, Zea mays
atgcaggagttccagtccatccctggcctggccgggcggctcttcggcggggccgcggcccccggcgacctccggcgcgcgcaggcggcgcagcatggccccggcggagcgcggtgcggcggtgcctcgcccgcggcgccggaggcggtgaagtgcccgcggtgcgagtctaccaacaccaagttctgctactacaacaactacaacctgtcgcagccgcgccacttctgcaagagctgccgccggtactggaccaagggcggcgtcctgcgcaacgtccccgtgggcggcggctgccgcaaggcgaagcgcgcctcgtcctcggcgtcggcgtcggcgtcggtgtcctcgcccgcctcgtcctcggcgccgtccacgcccgcgtcggccgacgcgggcaagaacccgcgccgcgcctcggcgtcctcgccctcgccccgctccaacagcggcagcgcgagccccacggccgccgccgcgacgaccccgaccccgaccgatccgaccacccccgccacgccgtcgtcgaacggcgtcgccttcacgggcagccaccactcgtcgaaccccttctccacgatcgacgtggcggccgcgccaccggcgccgatattcgccgaccaggcggcggcgctggcgtccctcttcgcgcctccccctccgccgccgctcccggtgttcagcttcgcggcggcgcagccgaaggaggaggaagcgcccaccaattcggagctgcagcaacacctcgccgcacaggcggcgccgtcgtcgtcggtctccgaggacatcatggcgccgttcgcatccctggacgccgctgggatattcgagctcggcgacgccgcgtcagccgcggcgtactggagcgccgggagctgctgggcggacgtccaggacccgagcttgtacctaccctag

SEQ ID NO: 36 - protein, 305 aa, Zea mays
MQEFQSIPGLAGRLFGGAAAPGDLRRAQAAQHGPGGARCGGASPAAPEAVKCPRCESTNTKFCYYNNYNLSQPRHFCKSCRRYWTKGGVLRNVPVGGGCRKAKRASSSASASASVSSPASSSAPSTPASADAGKNPRRASASSPSPRSNSGSASPTAAAATTPTPTDPTTPATPSSNGVAFTGSHHSSNPFSTIDVAAAPPAPIFADQAAALASLFAPPPPPPLPVFSFAAAQPKEEEAPTNSELQQHLAAQAAPSSSVSEDIMAPFASLDAAGIFELGDAASAAAYWSAGSCWADVQDPSLYLP

SEQ ID NO: 37 - DNA, 2080 bp, Zea mays
ctttcaggcgtcgccaactccacttgtcagatcttgcccgtatggtatggagctcgccggagcagcctcccctcacccgccgccggaatcccacgtggcgccgccccgcccacctccgcagccgccggaagaggatttatgtgaagatagaggggacatgagtgtcaccggcgaaaagccatgcacacatcgggaatcagatgttggtcagacaaatagttgtagccttaacaattccagtgagtgtgagaatcatacacccagcaacgacgaaatatcggaaccagagtccaatttggagatggccaagactgatcagggcggcgatgtgccgagcggagagaaggtcctgaagaagccagacaagatcctgccgtgccctcgctgcaacagcatggacacgaagttctgctactacaacaactacaacatcaagcagccaaggcatttctgcaagagctgtcagaggtactggaccgcaggcggaagcatgagaaacatccccgttggtgctgggaggcgcaagagcaagagctccagcgcgagttgccgcagcgtgttgattcccgttccaggcagcagtagtgtagctaatcctggcggagaggcttccctgtttccgttgtccgtaaaagcaaaccaagcagcagttagcttcgggcctgattcccccctctgcacctccatggcctccgtgctgaagatcggaggaggaggagagcaggttaagagctgcagccctgcctcagcagcagcacagcccaggaacggagaaacccagacccagacgtgcgcaccttcttctgctgctacaacaccatcagatggtccagggaatggattgcagaaaggagcagagagcgcacaccaaaaccaaaaccaaaaccaaaacggaatcattgggcacagcaacggagtcactccagtgcatcctataccgttcttccccggaccgcctttcgtgtacccctggagtccagcatggaacggcattcccgccatggcagcggcggtgtgcgcagccccagcgacagccgaagcagcgatttcatcagaacacggcaccgcgagcagcaccgtccagtggaacgtgccaccagcgatcgtgcccgtgctgccaccgggattctgcggcccgatcccagtcccggtaatcccgccgccttccgtctggccactgatcactccctggcccaacgcagcatggagcgcgccgtggctcgggcctagcgctagcgtgccgccggggtcatctcggagcagcggcagcagcacgtgctccgacagcggctgcggctccggctcccccgtcctgggaaagcactcgagggagtcgaggccgcagggcgacgagaaggcggaacgacggtgcctgtggatccccaagacgctccggatcgacgaccctgtcgaggccgccaagagctcgatctggacgacgctcgggatcgagcctggcgaccggggcatgttcaggccgttccagtcgaagcatggacggcagcaggagctgcaagcgtccggcgccgctcgcgccctgcaggccaacccggcggctctgtcgcgctcgcagtcttttcaggagacgacgtgattactacactacagagcagagcgttgctgaaacctgtgtggcattactattcagagacgattgtatttcaacttcaaggggggaaatgagagagagagcgcgagagagagattggcctgtttggttcactacctcagttgccacactttgcctaacttttctgactaaggttagttattcaattcgaacgactaacattaggcaaagtgtggcatatttagtcataaaccaaacatgccatatatgttctgtacaatatgattggcaacataggtgctgctgctgtacaaggcagcggacaccgttactgtaactgtaaccattggaagagtgcatcctcaaacagtgtgttgtttccatgtagagcgtgcccccaacatgttgggttggaggtgatcaggtttccagcgcgtgcgtgcttggttgtacaagaataactgtcaatagcttcccttcaaaaaaaaaaaaaaaaaaaaaaaaaaaaaa

SEQ ID NO: 38 - DNA, 1548 bp, Zea mays
atggagctcgccggagcagcctcccctcacccgccgccggaatcccacgtggcgccgccccgcccacctccgcagccgccggaagaggatttatgtgaagatagaggggacatgagtgtcaccggcgaaaagccatgcacacatcgggaatcagatgttggtcagacaaatagttgtagccttaacaattccagtgagtgtgagaatcatacacccagcaacgacgaaatatcggaaccagagtccaatttggagatggccaagactgatcagggcggcgatgtgccgagcggagagaaggtcctgaagaagccagacaagatcctgccgtgccctcgctgcaacagcatggacacgaagttctgctactacaacaactacaacatcaagcagccaaggcatttctgcaagagctgtcagaggtactggaccgcaggcggaagcatgagaaacatccccgttggtgctgggaggcgcaagagcaagagctccagcgcgagttgccgcagcgtgttgattcccgttccaggcagcagtagtgtagctaatcctggcggagaggcttccctgtttccgttgtccgtaaaagcaaaccaagcagcagttagcttcgggcctgattcccccctctgcacctccatggcctccgtgctgaagatcggaggaggaggagagcaggttaagagctgcagccctgcctcagcagcagcacagcccaggaacggagaaacccagacccagacgtgcgcaccttcttctgctgctacaacaccatcagatggtccagggaatggattgcagaaaggagcagagagcgcacaccaaaaccaaaaccaaaaccaaaacggaatcattgggcacagcaacggagtcactccagtgcatcctataccgttcttccccggaccgcctttcgtgtacccctggagtccagcatggaacggcattcccgccatggcagcggcggtgtgcgcagccccagcgacagccgaagcagcgatttcatcagaacacggcaccgcgagcagcaccgtccagtggaacgtgccaccagcgatcgtgcccgtgctgccaccgggattctgcggcccgatcccagtcccggtaatcccgccgccttccgtctggccactgatcactccctggcccaacgcagcatggagcgcgccgtggctcgggcctagcgctagcgtgccgccggggtcatctcggagcagcggcagcagcacgtgctccgacagcggctgcggctccggctcccccgtcctgggaaagcactcgagggagtcgaggccgcagggcgacgagaaggcggaacgacggtgcctgtggatccccaagacgctccggatcgacgaccctgtcgaggccgccaagagctcgatctggacgacgctcgggatcgagcctggcgaccggggcatgttcaggccgttccagtcgaagcatggacggcagcaggagctgcaagcgtccggcgccgctcgcgccctgcaggccaacccggcggctctgtcgcgctcgcagtcttttcaggagacgacgtga

SEQ ID NO: 39 - protein, 515 aa, Zea mays
MELAGAASPHPPPESHVAPPRPPPQPPEEDLCEDRGDMSVTGEKPCTHRESDVGQTNSCSLNNSSECENHTPSNDEISEPESNLEMAKTDQGGDVPSGEKVLKKPDKILPCPRCNSMDTKFCYYNNYNIKQPRHFCKSCQRYWTAGGSMRNIPVGAGRRKSKSSSASCRSVLIPVPGSSSVANPGGEASLFPLSVKANQAAVSFGPDSPLCTSMASVLKIGGGGEQVKSCSPASAAAQPRNGETQTQTCAPSSAATTPSDGPGNGLQKGAESAHQNQNQNQNGIIGHSNGVTPVHPIPFFPGPPFVYPWSPAWNGIPAMAAAVCAAPATAEAAISSEHGTASSTVQWNVPPAIVPVLPPGFCGPIPVPVIPPPSVWPLITPWPNAAWSAPWLGPSASVPPGSSRSSGSSTCSDSGCGSGSPVLGKHSRESRPQGDEKAERRCLWIPKTLRIDDPVEAAKSSIWTTLGIEPGDRGMFRPFQSKHGRQQELQASGAARALQANPAALSRSQSFQETT

SEQ ID NO: 40 - DNA, 1195 bp, Zea mays
ggctagctctagcagccctccgtatatcccagagagcgaggtggtgaccacgcgcgcgcccacagggccgggccagcgctagacactcagctgattgcactgagcagtgagcactagctaactctcggccgaatcgctgggcgtctattaagccgacgccaacgacgacggctcctcggcatgcgaggtcctgctcgcccgggggatggcgcctgcagcttcgatcctctcggtcaccgccgtcgccggttccaagcgtccggccgcttccgacgctgagctcccgctcctcgacctcgactcctcctcgctccaccagcagcaggtcagtagcagcgtgatcaatcctacgtacgtaccgtaccgacgacggcacgcacggcagttctgcgatgatcgatcactgacgtatatactacgcgtctgcggctgtgggctggcatggcagggtgacaaggctgggcgcaagggccaggaccaggaccaccaccagcagcagcagctggagtgcccgcgctgccgctccaccaacaccaagttctgctactacaacaactacagcacggcgcagccgcgccacttctgccgcgcgtgccgccgctactggacgcacggcggcacgctgcgcgacgtcccggttggcggggcctcgcgccgcgccggtgggggcggcaagcggcgcagggtctcctccgccgagacctcgtcgtcgtcgccgccggtgcctgcgtcgctcgcggacgcgtgcctgtccgacctcccgtccgtcttcccgttcctcagcgacggcagcttcttcccgcagctcgacctcggcgccgtcgtgcttgcaccgccggccttctcctcctcgtggcggtcggtggccccggacttctacgacgggctcgcgccgtggggcgacatcgccggcctcgacctcagctggacaccaccggggagcgccagccggtcgccgtcttgagggggctccggttttagcagtgcggcctcatgagccatcctagcaagcagtgtgttccgttttgatgagccatcctatcctatcctatccagctaggaaaatttaagcagctcgatcaaaattcgcctatgccttcactgttagatcagtgaataatcttgatctgtagttggagccaggatgtaagatctgtgcagtgatttatcatgacaataaaattaagtatgtgtc

SEQ ID NO: 41 - DNA, 618 bp, Zea mays
atggcgcctgcagcttcgatcctctcggtcaccgccgtcgccggttccaagcgtccggccgcttcagacgctgagctcccgcttctcggcctcgactcctcctcgctccaccagcagcagggtgacaaggctgggcgcaagggccaggaccaggaccaccagcagcagctggagtgcccgcgctgccgctccaccaacaccaagttctgctactacaacaactacagcacggcgcagccgcggcacttctgccgcgcgtgccgccgctactggacgcacggcggcacgctgcgcgacgtcccggttggcggggcctcgcgccgcgccggtaggggcggcaagcggcgcagggtctcctccgccgagacctcctcgtcgtcgtcgccgccgccgatgcctgcgtcgctcgcggacgcgtgcctgtccgacctcccgtccgtcttcccgttcctcagcgacggcagcttcttcccgcagctcgacctcggcgccgtcgtgcttgcaccgccggccttctcctcctcgtggcggtcggtggccccggacttctacgacgggctcgcgccgtggggcgacatcgccggcctcgacctcagctggacaccaccggggaactag

SEQ ID NO: 42 - protein, 205 aa, Zea mays
MAPAASILSVTAVAGSKRPAASDAELPLLGLDSSSLHQQQGDKAGRKGQDQDHQQQLECPRCRSTNTKFCYYNNYSTAQPRHFCRACRRYWTHGGTLRDVPVGGASRRAGRGGKRRRVSSAETSSSSSPPPMPASLADACLSDLPSVFPFLSDGSFFPQLDLGAVVLAPPAFSSSWRSVAPDFYDGLAPWGDIAGLDLSWTPPGN

SEQ ID NO: 43 - DNA, 1584 bp, Zea mays
tcttttcccatcactgacgacgagaggtggaggttctggccggagcagctgagaggtgcataaaaaatattgtaaaaaaataatcctccactgctagattggctattgggcaagagagagcggagaggggaagggaaggaggcataattccactcacactggccaaccacagggcacagggtagcattctctctcttcgtcctcaagaaatccagcggaagctcatcttcttcaccctcccttctctccacatctcccttcggactactactgcatccaaggagcaaagaattgtggggggtagcaaaggaattggaggtcttgcatggatgcagcccactggcagcagggcctagggctggtgaagcccatggaggagatgctgatggcggccaacgcgggcgccgcaaatccgagccaaagctcgaatccgaacccgccggcgccggcgccgtcgttggcacctggggggctcctgggcggtggcgcgccggcgcccctggcgggcgcgggtagtaccgagcggcgcgcgcggccgcagaaggagaaggcgctcaactgcccgcggtgcaactccaccaacaccaaattctgctactacaacaactacagcctccagcagccacgctacttctgcaagacgtgccgccgctactggacggagggcggatccctccgcaacgtccccgtgggcggcggctcccgcaagaacaagcgctcctcatcgtcggcgtcggcgtctgcctccacctccggctcggtcacgagctcgtccatggccagcacggcgggggccgggtccaagaacccgaagctggcgcacgagggcgcccacgacctcaacctggcgttcccgcaccacggtggcctgcacgcccccgagttcgcggcgttcccgagtctggagagcagcaacgtgtgcaacccgggcggcgggatgacgagcaacggtcggggcggcggcgcggggcccgcggtcggcgcgctctcggcaatggagctgttgcggagctccggctgctacatgccgctgcagatgccgatgccgatggcgatgccaggagattacacggcggcagggttcgcgctcggagagtaccgcacgccgccgcctccaccgtcacagagcgtgctcgggttctctctcgacgcgcacgggccggggtccggtgctaccgcggcggggtacggttccagcgcggggttgcagggcgtgccggagaacgcgggcaggttattgttccccttcgaagacttgaagccgccggttggctctgaaggtgggggtggtgcaaccggtggcgccagcgatggaaatagcagccatactcagtttgaccacaacaacaaggagcaaggcggcggcggcacgggtgccgggcacgacacgccggggttctggagcggcatgatcggcggcagtggcgcttcttggtaatggagaaacatgcacggccggggggcggcgccatgcatggctcatgcaaaggctccctgcgagcgtgcatgggcatgcagtagagacaagggttcaataagaggttggcattggtggtggttgccctgaaaaaaaaaaaaaaaaaa

SEQ ID NO: 44 - DNA, 1113 bp, Zea mays
atggatgcagcccactggcagcagggcctagggctggtgaagcccatggaggagatgctgatggcggccaacgcgggcgccgcaaatccgagccaaagctcgaatccgaacccgccggcgccggcgccgtcgttggcacctggggggctcctgggcggtggcgcgccggcgcccctggcgggcgcgggtagtaccgagcggcgcgcgcggccgcagaaggagaaggcgctcaactgcccgcggtgcaactccaccaacaccaaattctgctactacaacaactacagcctccagcagccacgctacttctgcaagacgtgccgccgctactggacggagggcggatccctccgcaacgtccccgtgggcggcggctcccgcaagaacaagcgctcctcatcgtcggcgtcggcgtctgcctccacctccggctcggtcacgagctcgtccatggccagcacggcgggggccgggtccaagaacccgaagctggcgcacgagggcgcccacgacctcaacctggcgttcccgcaccacggtggcctgcacgcccccgagttcgcggcgttcccgagtctggagagcagcaacgtgtgcaacccgggcggcgggatgacgagcaacggtcggggcggcggcgcggggcccgcggtcggcgcgctctcggcaatggagctgttgcggagctccggctgctacatgccgctgcagatgccgatgccgatggcgatgccaggagattacacggcggcagggttcgcgctcggagagtaccgcacgccgccgcctccaccgtcacagagcgtgctcgggttctctctcgacgcgcacgggccggggtccggtgctaccgcggcggggtacggttccagcgcggggttgcagggcgtgccggagaacgcgggcaggttattgttccccttcgaagacttgaagccgccggttggctctgaaggtgggggtggtgcaaccggtggcgccagcgatggaaatagcagccatactcagtttgaccacaacaacaaggagcaaggcggcggcggcacgggtgccgggcacgacacgccggggttctggagcggcatgatcggcggcagtggcgcttcttggtaa

SEQ ID NO: 45 - protein, 370 aa, Zea mays
MDAAHWQQGLGLVKPMEEMLMAANAGAANPSQSSNPNPPAPAPSLAPGGLLGGGAPAPLAGAGSTERRARPQKEKALNCPRCNSTNTKFCYYNNYSLQQPRYFCKTCRRYWTEGGSLRNVPVGGGSRKNKRSSSSASASASTSGSVTSSSMASTAGAGSKNPKLAHEGAHDLNLAFPHHGGLHAPEFAAFPSLESSNVCNPGGGMTSNGRGGGAGPAVGALSAMELLRSSGCYMPLQMPMPMAMPGDYTAAGFALGEYRTPPPPPSQSVLGFSLDAHGPGSGATAAGYGSSAGLQGVPENAGRLLFPFEDLKPPVGSEGGGGATGGASDGNSSHTQFDHNNKEQGGGGTGAGHDTPGFWSGMIGGSGASW

SEQ ID NO: 46 - DNA, 1913 bp, Zea mays
catccgttcgtttgtttgcccttttttgccggcctcctcgtctcttccggttccggctacaagtgaagtgagctcggccgtctgttgcctagcgtgctgcttcgttcgtcgttggcttgcagcagcttgccgaagagaagaaagcagcaggcccggagctttctgtttgtttggatccgagtgagaagaagatggtggccgagtgccacggaggaggaggagcagcagcaggaggaggggactttctcatcaagctcttcgggatgaccatccccgtgccggagtgcggcgacgccaaggatcttcagcagagcaagagcaacaggtggaccgagcaggatcaggattcccatggcctggagaccgcgcccgcgcccgcgcacacggacacctccgacccgtccccgcagccggaggtcgtggacgccgaggaccccacggagacgcaacagaggccctgcaacggcgacggcgacggcgatgctgccggccagagggagaagctgaagaagcccgacaaggtgctgccgtgcccgcgctgcaacagcatggacaccaagttctgctacttcaacaactacaacgtcaaccagccgcgccacttctgcaagaactgccagcggtactggacggccggtggcgccatgcgcaacgtgcccgtgggggccggccgccgcaagaacaagaacgccgccgcctcgcacttcttccagagggtccgggcgaccaacgccacggtgctcagcttcgggggccatggcggcgcgcctcctgctgcccgcttggacctggacctcgccgagcagctgagccaccagctggccccggtcaggagcgccggcgacgcgggccgcccttgcagcgaaggatcgagcagcagggacggcaacggcagcaggtcttctgtagacgaagctgcagcaaacgcagacgacgggtcagtgcagcagcacccaggcccagcaagcatgaacagcagcggggcaaccgtgtggccgccgtacagctgcgccccagctccggcggcgtacttcccccagggcatcgcgattccgatctacccggccgcaccggcctactggggctgcatggttcccggagcttggagcctgccatggccggtgcagcacccgtcgtcgtcgtcgtcgcccaccaccaccagtgctccttcagtcgtctcgtcatccggggcagctgacgactcatcatcccacgcgctgggcaagcgcccccgggaccgggagggtgacgatgggagaaacggcggcaacgccaaggtgtgggcgcccaagagcatccggatagacgacgtggacgaggtggccaggagctcaatctggtccctcgtcgggatcaaaggcgaccagacgaagcagcaggacgcagcagacgaccacgccggtgggcacagcaagcagctcgggacggtgttcgagcccaagcgcggcgaggccaccaagaaggccatgatgacaagctcgccgctccttcacgcgaaccccgttgcgctcacgcgctccgtggccttccaggaggggtcttgattcttcttcaagtacatccatcagcagcagcaggaatttcctcatgcagcgttccttctcatctgaacttctgaactgaacatgagctgatcagcctgacagtacgtactgaattttgtttttatataaatcatcataggattgtaatatctatctatatgctgagtgggaaacttagctagcgtatgtgtaagtggacttgtaatataggcggcacgcaggatgtacatagaattaattagctatatgggtttgagaaagaccaggagatcaataagaggtccctgggtgtagataaataacattaggggaaggaatgtattgcttcaggtatttcactcttcaaaaaaaaaaaaaaaaaaaaaaaaa

SEQ ID NO: 47 - DNA, 1353 bp, Zea mays
atggtggccgagtgccacggaggaggaggagcagcagcaggaggaggggactttctcatcaagctcttcgggatgaccatccccgtgccggagtgcggcgacgccaaggatcttcagcagagcaagagcaacaggtggaccgagcaggatcaggattcccatggcctggagaccgcgcccgcgcccgcgcacacggacacctccgacccgtccccgcagccggaggtcgtggacgccgaggaccccacggagacgcaacagaggccctgcaacggcgacggcgacggcgatgctgccggccagagggagaagctgaagaagcccgacaaggtgctgccgtgcccgcgctgcaacagcatggacaccaagttctgctacttcaacaactacaacgtcaaccagccgcgccacttctgcaagaactgccagcggtactggacggccggtggcgccatgcgcaacgtgcccgtgggggccggccgccgcaagaacaagaacgccgccgcctcgcacttcttccagagggtccgggcgaccaacgccacggtgctcagcttcgggggccatggcggcgcgcctcctgctgcccgcttggacctggacctcgccgagcagctgagccaccagctggccccggtcaggagcgccggcgacgcgggccgcccttgcagcgaaggatcgagcagcagggacggcaacggcagcaggtcttctgtagacgaagctgcagcaaacgcagacgacgggtcagtgcagcagcacccaggcccagcaagcatgaacagcagcggggcaaccgtgtggccgccgtacagctgcgccccagctccggcggcgtacttcccccagggcatcgcgattccgatctacccggccgcaccggcctactggggctgcatggttcccggagcttggagcctgccatggccggtgcagcacccgtcgtcgtcgtcgtcgcccaccaccaccagtgctccttcagtcgtctcgtcatccggggcagctgacgactcatcatcccacgcgctgggcaagcgcccccgggaccgggagggtgacgatgggagaaacggcggcaacgccaaggtgtgggcgcccaagagcatccggatagacgacgtggacgaggtggccaggagctcaatctggtccctcgtcgggatcaaaggcgaccagacgaagcagcaggacgcagcagacgaccacgccggtgggcacagcaagcagctcgggacggtgttcgagcccaagcgcggcgaggccaccaagaaggccatgatgacaagctcgccgctccttcacgcgaaccccgttgcgctcacgcgctccgtggccttccaggaggggtcttga

SEQ ID NO: 48 - protein, 450 aa, Zea mays
MVAECHGGGGAAAGGGDFLIKLFGMTIPVPECGDAKDLQQSKSNRWTEQDQDSHGLETAPAPAHTDTSDPSPQPEVVDAEDPTETQQRPCNGDGDGDAAGQREKLKKPDKVLPCPRCNSMDTKFCYFNNYNVNQPRHFCKNCQRYWTAGGAMRNVPVGAGRRKNKNAAASHFFQRVRATNATVLSFGGHGGAPPAARLDLDLAEQLSHQLAPVRSAGDAGRPCSEGSSSRDGNGSRSSVDEAAANADDGSVQQHPGPASMNSSGATVWPPYSCAPAPAAYFPQGIAIPIYPAAPAYWGCMVPGAWSLPWPVQHPSSSSSPTTTSAPSVVSSSGAADDSSSHALGKRPRDREGDDGRNGGNAKVWAPKSIRIDDVDEVARSSIWSLVGIKGDQTKQQDAADDHAGGHSKQLGTVFEPKRGEATKKAMMTSSPLLHANPVALTRSVAFQEGS

SEQ ID NO: 49 - DNA, 1781 bp, Zea mays
cccacgcttccgtccatccagactccagagagataaaatctaattcctggacctggagtgcagaccgatcgccgccgccgccgcgttgtttcttgtgtgcgcagctgtggtgatggtgcggtaaaagaaggctgctccatcgctccaacaaaaaaaaaaggtcgagatcgcacgcggcaggcgacaagcagtttccaaagagaagggaagcctagaggaagggagctagagatgggggagggcagagcaggagacggcctcatcaagctgttcgggaagaccatccccgtgccggagacggccgccgtcggcgaggctgccaaggacatccaacaaagcggcggcagcggcagcggcacgactgatccgaaagggcaagagaacaacgttcaggactccacaggctcgcctccgcagcaggaggtcgcggacaccgaagactcgtcggcagacaaacagcagggcgaggcgggcaacccaaaggagaagctcaagaagcccgacaagatcctgccgtgcccgcggtgcaacagcatggacaccaagttctgctactacaacaactacaacatcaaccagccgcgccacttctgcaagaactgccagaggtactggactgccggcggtgccatgcgcaacgtgcccgtgggcgcaggccggcgcaagagcaagagcgcgtcggccacttcccacttcctccagagggtcagggccggtctgcccgtcgacccgctcgtctgcgcggcagccaagactaacggcacggtgctcagcttcggctctgccatgtccagcttagacctcacggagcagatgaaacagctcaaggagaagctcgtcccgatagcgggggacgagcgctcagttggctctcgcaatcaaggaccttctgccaaggcagaagacccggaccggaaggaaaatgttacagcagataaatccgcgagagttgttcagcatccatgcatgacgaacggggtggccatgtggccatttagctgcgcgccaccagtaccggcctctgcctgttacggcccaggcagcatcgcaatcccgttctacccggcagctgctgctgctgctgcctactggggttgcatggttccaggagcttggagtggcgcatggccgcctcactccggccagtccgagacgggctcatccattacctccgcctctccagcagcatccaccaagtccaacatctgcttcacgccaggaaagcaccctagagaccgcgacgaggaaggaggcgccaaaggaaatggcaaggtgtgggtgcccaagacgatccggatcgacgacgtggacgaggtggccaggagctctatcctgtcgctaatcgggatcggcggcgacaaggcaggcaaagatggcggcagaggctgcaagctcgcaagggtttttgagcagaacgaagaggcggcaaggacggcaactcctcactcggcagccatcagcggcttgccgttcttgcaggggaacccagctgcgctctcgcggtcactgaccttccaggaggggtcttgagcctctcgctcgattcacagctgagaattttgtacattagagggttgtatgtaatctaatctaggtggggggtggggctcatagctgccagcggacattgaaactatacaatagatagatatttgtagatagccaatatatatgggtcgggggaggaaaaaaaggataatgagagcaagtccccttatttgtatatattataatggattggaatatttggttaaaaaaaaaaaaaaaaaaaaaaaaaaaaa

SEQ ID NO: 50 - DNA, 1299 bp, Zea mays
atgggggagggcagagcaggagacggcctcatcaagctgttcgggaagaccatccccgtgccggagacggccgccgtcggcgaggctgccaaggacatccaacaaagcggcggcagcggcagcggcacgactgatccgaaagggcaagagaacaacgttcaggactccacaggctcgcctccgcagcaggaggtcgcggacaccgaagactcgtcggcagacaaacagcagggcgaggcgggcaacccaaaggagaagctcaagaagcccgacaagatcctgccgtgcccgcggtgcaacagcatggacaccaagttctgctactacaacaactacaacatcaaccagccgcgccacttctgcaagaactgccagaggtactggactgccggcggtgccatgcgcaacgtgcccgtgggcgcaggccggcgcaagagcaagagcgcgtcggccacttcccacttcctccagagggtcagggccggtctgcccgtcgacccgctcgtctgcgcggcagccaagactaacggcacggtgctcagcttcggctctgccatgtccagcttagacctcacggagcagatgaaacagctcaaggagaagctcgtcccgatagcgggggacgagcgctcagttggctctcgcaatcaaggaccttctgccaaggcagaagacccggaccggaaggaaaatgttacagcagataaatccgcgagagttgttcagcatccatgcatgacgaacggggtggccatgtggccatttagctgcgcgccaccagtaccggcctctgcctgttacggcccaggcagcatcgcaatcccgttctacccggcagctgctgctgctgctgcctactggggttgcatggttccaggagcttggagtggcgcatggccgcctcactccggccagtccgagacgggctcatccattacctccgcctctccagcagcatccaccaagtccaacatctgcttcacgccaggaaagcaccctagagaccgcgacgaggaaggaggcgccaaaggaaatggcaaggtgtgggtgcccaagacgatccggatcgacgacgtggacgaggtggccaggagctctatcctgtcgctaatcgggatcggcggcgacaaggcaggcaaagatggcggcagaggctgcaagctcgcaagggtttttgagcagaacgaagaggcggcaaggacggcaactcctcactcggcagccatcagcggcttgccgttcttgcaggggaacccagctgcgctctcgcggtcactgaccttccaggaggggtcttga

SEQ ID NO: 51 - protein, 432 aa, Zea mays
MGEGRAGDGLIKLFGKTIPVPETAAVGEAAKDIQQSGGSGSGTTDPKGQENNVQDSTGSPPQQEVADTEDSSADKQQGEAGNPKEKLKKPDKILPCPRCNSMDTKFCYYNNYNINQPRHFCKNCQRYWTAGGAMRNVPVGAGRRKSKSASATSHFLQRVRAGLPVDPLVCAAAKTNGTVLSFGSAMSSLDLTEQMKQLKEKLVPIAGDERSVGSRNQGPSAKAEDPDRKENVTADKSARVVQHPCMTNGVAMWPFSCAPPVPASACYGPGSIAIPFYPAAAAAAAYWGCMVPGAWSGAWPPHSGQSETGSSITSASPAASTKSNICFTPGKHPRDRDEEGGAKGNGKVWVPKTIRIDDVDEVARSSILSLIGIGGDKAGKDGGRGCKLARVFEQNEEAARTATPHSAAISGLPFLQGNPAALSRSLTFQEGS

SEQ ID NO: 52 - DNA, 1511 bp, Zea mays
gcacgccccggcccctttgttttgtagctcttgtcagcgaaccttccccctgaaaaccccacccacccttcctctccgatcctcctagtagcgcctcacttgcttcattccgatccattccaccacccatctccctcctcagctcccctctccgctctccctccctcgctcgcgctctccacgcggatcaccggaggctagctagcaagctcgaccggcgcggccgatctagctcgcgcttgttggccgcttcagcttagcagcggcggcacgtcgtcaacctgtgctgtgagaaagactagtagagagagatacgtgtaaggatggctggcgcggggggtgcgacggctgtgaagcagccggcggcgggggcgcccgacgctggggccagcaggagcggggtcggggctggcgccgccggggcgccggtcgccgacccgcgcgccgaggcgctgcggtgcccgcgctgcgactcggccaacaccaagttctgctactacaacaactactcgctgtcgcagccgcggcacttctgcaaggcgtgcaagcgctactggacgcgcgggggcacgctccgcaacgtccccgtcggcgggggctgccgcaagaacaagcgctccaggagcagcggcgggcccggcgccggcgggaggaacgggtcctccgctgctgctgctgctgctgccgccgccgccgccgtcacgtcctcctcggcgccgtcgacgctgtccctcccgctgcatacggggtccctgccgtcgttgtcctcggcgctggggctgcccgggggcgcctcgctcgcgtcgctcctcctcgggaccgctggctctggcggtgaccacctcggcctcttccaggccgccatgcagtcggtggtctcctcggaggcgaccgcctacgagatgcagcagcagcagcagcagcagacgcaggtggaccacctgctaggcctcggctacggaggcgccggcgccggcgccggcgcgcagatccacctcaagccgtggatgcacgaggcacccggtgctggcgcgggcgggatcatggacagcttctacgcgccgctgctgtccagctccctcgtgccgggcctggaggagctgcacgtcaaggcggaggtcgccggtgccggggatcaccagcagaagcccgcgcccggggaccagcagagcgccagctgggagctgccgacgccgtcgtcgtccaacgtcgacgccaacgtcatcgcatctgacgcgctcatggccgccgccgccgcgtccatgaaccccggggttagctccgccacagcacccacggcgccaaccgtcccctcctccttcatgtactggggcaacggcggtattggcggcgctgccgcggcgtggccagacctcaccaactgcggatcttccattgccacgttcttctagccgtctggctgtcccgcactcccgctggcctcgttaacttcgttttttttaccttaattttctggttaattttttcttgactaaaaaaaaaaaaaaaaa

SEQ ID NO: 53 - DNA, 1089 bp, Zea mays
atggctggcgcggggggtgcgacggctgtgaagcagccggcggcgggggcgcccgacgctggggccagcaggagcggggtcggggctggcgccgccggggcgccggtcgccgacccgcgcgccgaggcgctgcggtgcccgcgctgcgactcggccaacaccaagttctgctactacaacaactactcgctgtcgcagccgcggcacttctgcaaggcgtgcaagcgctactggacgcgcgggggcacgctccgcaacgtccccgtcggcgggggctgccgcaagaacaagcgctccaggagcagcggcgggcccggcgccggcgggaggaacgggtcctccgctgctgctgctgctgctgccgccgccgccgccgtcacgtcctcctcggcgccgtcgacgctgtccctcccgctgcatacggggtccctgccgtcgttgtcctcggcgctggggctgcccgggggcgcctcgctcgcgtcgctcctcctcgggaccgctggctctggcggtgaccacctcggcctcttccaggccgccatgcagtcggtggtctcctcggaggcgaccgcctacgagatgcagcagcagcagcagcagcagacgcaggtggaccacctgctaggcctcggctacggaggcgccggcgccggcgccggcgcgcagatccacctcaagccgtggatgcacgaggcacccggtgctggcgcgggcgggatcatggacagcttctacgcgccgctgctgtccagctccctcgtgccgggcctggaggagctgcacgtcaaggcggaggtcgccggtgccggggatcaccagcagaagcccgcgcccggggaccagcagagcgccagctgggagctgccgacgccgtcgtcgtccaacgtcgacgccaacgtcatcgcatctgacgcgctcatggccgccgccgccgcgtccatgaaccccggggttagctccgccacagcacccacggcgccaaccgtcccctcctccttcatgtactggggcaacggcggtattggcggcgctgccgcggcgtggccagacctcaccaactgcggatcttccattgccacgttcttctag

SEQ ID NO: 54 - protein, 362 aa, Zea mays
MAGAGGATAVKQPAAGAPDAGASRSGVGAGAAGAPVADPRAEALRCPRCDSANTKFCYYNNYSLSQPRHFCKACKRYWTRGGTLRNVPVGGGCRKNKRSRSSGGPGAGGRNGSSAAAAAAAAAAAVTSSSAPSTLSLPLHTGSLPSLSSALGLPGGASLASLLLGTAGSGGDHLGLFQAAMQSVVSSEATAYEMQQQQQQQTQVDHLLGLGYGGAGAGAGAQIHLKPWMHEAPGAGAGGIMDSFYAPLLSSSLVPGLEELHVKAEVAGAGDHQQKPAPGDQQSASWELPTPSSSNVDANVIASDALMAAAAASMNPGVSSATAPTAPTVPSSFMYWGNGGIGGAAAAWPDLTNCGSSIATFF

SEQ ID NO: 55 - DNA, 1444 bp, Zea mays
gcaccagcgccgatgcccataaacttcgagagcgaccaccagctgaaacaccccgtcatctcctactgtctatgccatacggcggccgccgcgacgctccctaggctccgagtcactccgagctgggaaggaagagcccgcgttgctcactcgctcgctcgcatgcagctttcccacacggcgccacgcatgcaggagcccgggcgccggcccgtaccgccgttcgcgggcgtcgacctccgccgccccaggggctaccccgctccggcggcgaaggaggcggaggaggagccggccccagctccggcgccgggcggcgacccgtgcccgcggtgcgggtcgcgggacaccaagttctgctactacaacaactacaacacgtcgcagccgcgccacttctgcaagtcgtgccgccgctactggaccaagggcgggtccctccgtaacgtccccgtcgggggcggcacccgcaagagcggctcctcgtcctcggcctcggcaacggcaacgccaacctcgacgggcgccgcacccaagagcccgaggtcgtccaagaactccaagcgtcgccgcgtcggccccgccccggccccggccccggccccggaccccgcccctgccaccgccaccgccaccgctgacgtcgcgaacacggcaccgtcgacggaagcctcagcagcaacgcgtgccgcctcggagaagccagccgcgacggaacccgcagcggcaacgggtgccgcctcggagaagccggccgcgacggagctcgcagcagcggttgtctccacggagaaaccgccggcggccgtcggcggtttcacggacacgtctacggcgcccgagaccgggctcgcagatgtctgcagcggcggcgtgaaggagcttgtgccggacccgggccacttcgagtggccgtcgggctgcgatctggggtcctactggggcaccagcgtgttcgccgacaccgacccggcgctgttcctgaacctgccgtgagccgtgacaacgccacggcgtccatgctcaactaatcaaccatcacacgacccacgagatagatgagatgtgaaccgagcacgctgagcgtcaagctcttgaaggcttgaagctgtgctgtagtttcctgagcttactagcgatctctgcttgtctctgatctgataatctcttactagtcgccgtttgtgctccgatctagtaacggctaacatggtcggctgttacaagtgcatgaacctgtgcttgatcttgagctaaatcatggtgtttttgaggctatgatcgaggatcacaagaacagctgcaaggagaacggcgacttgtaagtcgtgtcgttgtaactcttggtttggttggaggcatatattctgaagccgtctaaagcatgccgcgcaaaaaaaaaaaaaaaaaaaaaaaaaaaaaaaaaaaaaaaaaaaaaaaaaaa

SEQ ID NO: 56 - DNA, 984 bp, Zea mays
atgcccataaacttcgagagcgaccaccagctgaaacaccccgtcatctcctactgtctatgccatacggcggccgccgcgacgctccctaggctccgagtcactccgagctgggaaggaagagcccgcgttgctcactcgctcgctcgcatgcagctttcccacacggcgccacgcatgcaggagcccgggcgccggcccgtaccgccgttcgcgggcgtcgacctccgccgccccaggggctaccccgctccggcggcgaaggaggcggaggaggagccggccccagctccggcgccgggcggcgacccgtgcccgcggtgcgggtcgcgggacaccaagttctgctactacaacaactacaacacgtcgcagccgcgccacttctgcaagtcgtgccgccgctactggaccaagggcgggtccctccgtaacgtccccgtcgggggcggcacccgcaagagcggctcctcgtcctcggcctcggcaacggcaacgccaacctcgacgggcgccgcacccaagagcccgaggtcgtccaagaactccaagcgtcgccgcgtcggccccgccccggccccggccccggccccggaccccgcccctgccaccgccaccgccaccgctgacgtcgcgaacacggcaccgtcgacggaagcctcagcagcaacgcgtgccgcctcggagaagccagccgcgacggaacccgcagcggcaacgggtgccgcctcggagaagccggccgcgacggagctcgcagcagcggttgtctccacggagaaaccgccggcggccgtcggcggtttcacggacacgtctacggcgcccgagaccgggctcgcagatgtctgcagcggcggcgtgaaggagcttgtgccggacccgggccacttcgagtggccgtcgggctgcgatctggggtcctactggggcaccagcgtgttcgccgacaccgacccggcgctgttcctgaacctgccgtga

SEQ ID NO: 57 - protein, 327 aa, Zea  mays
MPINFESDHQLKHPVISYCLCHTAAAATLPRLRVTPSWEGRARVAHSLARMQLSHTAPRMQEPGRRPVPPFAGVDLRRPRGYPAPAAKEAEEEPAPAPAPGGDPCPRCGSRDTKFCYYNNYNTSQPRHFCKSCRRYWTKGGSLRNVPVGGGTRKSGSSSSASATATPTSTGAAPKSPRSSKNSKRRRVGPAPAPAPAPDPAPATATATADVANTAPSTEASAATRAASEKPAATEPAAATGAASEKPAATELAAAVVSTEKPPAAVGGFTDTSTAPETGLADVCSGGVKELVPDPGHFEWPSGCDLGSYWGTSVFADTDPALFLNLP

SEQ ID NO: 58 - DNA, 1294 bp, Zea mays
cgaagctctcctcgccgcactcgcgcctgctcgaccgctccaacaagaccctctcgcgccaccgcagcagcgtcgtcgtcgctacgcggatgctgtcttctcaccgcgagagcctgctgccctacgcgccggggcggcgggcggcggtgctgctcgaccaccggcggtaccgcccgaacgtcgaggtggcgcccagctgcccgcgctgcgactcgcccaacaccaagttctgctactacaacaactacagcctcagccagccccgctacttctgcaagggctgccgccgctactggaccaagggcggctccctccgcaacgtgcccgtcggcggcgggtgccggaagaaccgcagcaggggcaagccggtccgagccgtggccgtcgattccgtggcgcccagcagcggtggcgcggcggcgttcacgcaccgggcgtcggcgtcgtcgtcgtcggcgtcccccgccacgctgcggccggacatgctactggaaggcatgctcggcagcccaagcgagctgtgccagctgacggtgacggacgacgacgcagcggaaaacccggctgtggcgcccgatggacccaggatcgacctggcgttgctgtacgccaagttcttgaaccaacagccgccggcgcccgagcggcgcgccgtcctgccggaatcgctcgtcgtcgacaccttgagcgggtcgtcgtcgagcgacgtgggtcccgtcgtcccatcgcgggatcatcagccgttcacgacgcaagacgacgggctgggcgagctgtccgcggcggcgagcgcagagccgagcgctcctccgccacagtcgtcgtcgtgtgcggaggcgcttattggggcgttcggcatggaccagcgctgctacgactctctgggtttgccaccagacggcggggatctagtcctaccgtcgacgtggcctcttcttggggccaagtacgagctgttcgatccgctacctgaggacgccgtgagccttcaggacggcttcgccggcgacgaggacgtgtggagcggcgccttggcttgtcaagggctggaagcagctctctgcaggccgtaattttttttaactcgcctcttgattttcaccgtgcttgtgattgttttatgcgctcttttctttttcctggttgtgatcgtgttcaggagtaatttatatagagaccatatatacagcatgtccagatcatgcattatgatggatcaacgggtatacatgtgagatatagaaagaacttaataaaattttgacattttgtttgacaaaaaaaaaaaaaaaaa

SEQ ID NO: 59 - DNA, 984 bp, Zea mays
atgctgtcttctcaccgcgagagcctgctgccctacgcgccggggcggcgggcggcggtgctgctcgaccaccggcggtaccgcccgaacgtcgaggtggcgcccagctgcccgcgctgcgactcgcccaacaccaagttctgctactacaacaactacagcctcagccagccccgctacttctgcaagggctgccgccgctactggaccaagggcggctccctccgcaacgtgcccgtcggcggcgggtgccggaagaaccgcagcaggggcaagccggtccgagccgtggccgtcgattccgtggcgcccagcagcggtggcgcggcggcgttcacgcaccgggcgtcggcgtcgtcgtcgtcggcgtcccccgccacgctgcggccggacatgctactggaaggcatgctcggcagcccaagcgagctgtgccagctgacggtgacggacgacgacgcagcggaaaacccggctgtggcgcccgatggacccaggatcgacctggcgttgctgtacgccaagttcttgaaccaacagccgccggcgcccgagcggcgcgccgtcctgccggaatcgctcgtcgtcgacaccttgagcgggtcgtcgtcgagcgacgtgggtcccgtcgtcccatcgcgggatcatcagccgttcacgacgcaagacgacgggctgggcgagctgtccgcggcggcgagcgcagagccgagcgctcctccgccacagtcgtcgtcgtgtgcggaggcgcttattggggcgttcggcatggaccagcgctgctacgactctctgggtttgccaccagacggcggggatctagtcctaccgtcgacgtggcctcttcttggggccaagtacgagctgttcgatccgctacctgaggacgccgtgagccttcaggacggcttcgccggcgacgaggacgtgtggagcggcgccttggcttgtcaagggctggaagcagctctctgcaggccgtaa

SEQ ID NO: 60 - protein, 327 aa, Zea mays
MLSSHRESLLPYAPGRRAAVLLDHRRYRPNVEVAPSCPRCDSPNTKFCYYNNYSLSQPRYFCKGCRRYWTKGGSLRNVPVGGGCRKNRSRGKPVRAVAVDSVAPSSGGAAAFTHRASASSSSASPATLRPDMLLEGMLGSPSELCQLTVTDDDAAENPAVAPDGPRIDLALLYAKFLNQQPPAPERRAVLPESLVVDTLSGSSSSDVGPVVPSRDHQPFTTQDDGLGELSAAASAEPSAPPPQSSSCAEALIGAFGMDQRCYDSLGLPPDGGDLVLPSTWPLLGAKYELFDPLPEDAVSLQDGFAGDEDVWSGALACQGLEAALCRP

SEQ ID NO: 61 - DNA, 1079 bp, Zea mays
ctcggtcgcattctcttcctctcctagtccttcccatgatcccatcccatatatcccagccaggtcacccagagagcgagaagctgattgcgccgcgccctagctagcgctctcggccgcatcgccggacgtctattaacccgccgccgctcgtcctcggcatgcgcggttctgctcgcctgcgagatggcgcctgcagtttccatcctctcggccaccgcctccgccaagcgaaagcgccccgccacttccgacgctgatgagctcccgcacgacgactcctccgcgccccaccagcaggtgcagggccagggccagcaaccgcggcagcagcagcagcttgagtgcccgcgctgccgctccaccaacaccaagttctgctactacaacaactacagcacggcgcagccgcgccacttctgccgcgcgtgccgccgctactggacgcacgggggcacgctgcgcgacgtccccgtcggcggcgcctcgcgccgcgccgccactggcggcggcggcggcaagcggcgcagggtctccgccgagccctcatccccgccgccgtcggtcgcggacgcgtgcctgccgtccgccttcccgttcctcagcgacggcagcttcttcccgcagctcgacctcgtcggcggcgttgcgcttgcacccccggccttctcctcctcgtggcagtcggtgctggtcccggacttgtacgacgggctcgcgccgtgggacgacggagcaacggcggccgcgtggggcgacatcggtggcctcgacctcagctggacactgccggggaactgagtgccagccgccgtcgcgcgtcttgaggggctccggttttagcagtgcggcctcgtcaggttcagttcagttcagttcctagcatgcagagctctcacaattctcctatgtcgcctccactgtttcttctcgtccttactgcgaatgaaatggtggggctgctagttagatcagtcaatcttgatctgtagctggagccagagtgtaagatctgtgcagtgatttatcgtgacaataaaattaagtttagtttgtgtcaaaaaaaaaaaaaaaaaaaa

SEQ ID NO: 62 - DNA, 618 bp, Zea mays
atggcgcctgcagtttccatcctctcggccaccgcctccgccaagcgaaagcgccccgccacttccgacgctgatgagctcccgcacgacgactcctccgcgccccaccagcaggtgcagggccagggccagcaaccgcggcagcagcagcagcttgagtgcccgcgctgccgctccaccaacaccaagttctgctactacaacaactacagcacggcgcagccgcgccacttctgccgcgcgtgccgccgctactggacgcacgggggcacgctgcgcgacgtccccgtcggcggcgcctcgcgccgcgccgccactggcggcggcggcggcaagcggcgcagggtctccgccgagccctcatccccgccgccgtcggtcgcggacgcgtgcctgccgtccgccttcccgttcctcagcgacggcagcttcttcccgcagctcgacctcgtcggcggcgttgcgcttgcacccccggccttctcctcctcgtggcagtcggtgctggtcccggacttgtacgacgggctcgcgccgtgggacgacggagcaacggcggccgctctagaggatccaagcttacgtacgcgtgcatgcgacgtcatagctcttctatag

SEQ ID NO: 63 - protein, 205 aa, Zea mays
MAPAVSILSATASAKRKRPATSDADELPHDDSSAPHQQVQGQGQQPRQQQQLECPRCRSTNTKFCYYNNYSTAQPRHFCRACRRYWTHGGTLRDVPVGGASRRAATGGGGGKRRRVSAEPSSPPPSVADACLPSAFPFLSDGSFFPQLDLVGGVALAPPAFSSSWQSVLVPDLYDGLAPWDDGATAAALEDPSLRTRACDVIALL

SEQ ID NO: 64 - DNA, 1210 bp, Zea mays
gcgagtggagctcttgcgtcttgccactttgccagttgccacgctccgggctcctcggcaaacacacagcaccagctaagctagctcacgcccgccgaggccatatccttcccagcacgccgtagacgccgacgcttgacgccatgcaggagttccaatccatcccggggctggcggggcggctgttcggcggggcagcggccgccggcctccggcgcgcgcaggggcagtgcggcggcgggggtgcctcggccgcggcggccgtgaagtgcccgcggtgcgagtccaccaacaccaagttctgctactacaacaactacaacctgtcgcagccgcgccacttctgcaagggctgccggcggtactggaccaagggcggcgtcctgcgcaacgtgcccgtgggcgggggctgccgcaaggccaagcgctcgtcggcgccgtccacgcccacgcccacgcccacgtcggcggtgggcgacgccaagagcccgcgccgcgcctcggcgtcctcgccccgcggctccatcagcagcggcagcggcagcgcgagccccacgcccaacggcttcgccttctcctccacggccgacgtggtggcgccgccgccggcgccgatattcgccgatcaggcggcggcgctggcgtctctcttcgcgcctccgccgccgctcccggcgttcagcttcgtggcggcgcaggctaaggaggaaacgtcctccacccccgctggcttcgcggcgccgtactcctcagtatccgaggacatggcaccgttcgcgtccctggacgccgctgggatgttcgagatcggcgaggacgcgtcggccgcggcggcgtactggaacgccgggagctgctggacggacgtgccggacccgagcatgatgatgtacctgctaccctaggctgtttgttttaggtacttgtcacgatcgcttgattaaggttaagccgtggttaattaagtgcttcttattaaacgacgacgaccacgagctacgtctccctactagtatattatatgtccacgctccacccgccggatgccttcctctgcagcgtcgatcttgtgtttgtttattggtgatttgtggcttcacagaccgcgtgtaagaaaagctaacgtctgtgttgcactcgtaagctgatcattgccctagcttgcaattgcaacgctgcgatgaaattattcgcaaaaaaaaaaaaaaaa

SEQ ID NO: 65 - DNA, 762 bp, Zea mays
atgcaggagttccaatccatcccggggctggcggggcggctgttcggcggggcagcggccgccggcctccggcgcgcgcaggggcagtgcggcggcgggggtgcctcggccgcggcggccgtgaagtgcccgcggtgcgagtccaccaacaccaagttctgctactacaacaactacaacctgtcgcagccgcgccacttctgcaagggctgccggcggtactggaccaagggcggcgtcctgcgcaacgtgcccgtgggcgggggctgccgcaaggccaagcgctcgtcggcgccgtccacgcccacgcccacgcccacgtcggcggtgggcgacgccaagagcccgcgccgcgcctcggcgtcctcgccccgcggctccatcagcagcggcagcggcagcgcgagccccacgcccaacggcttcgccttctcctccacggccgacgtggtggcgccgccgccggcgccgatattcgccgatcaggcggcggcgctggcgtctctcttcgcgcctccgccgccgctcccggcgttcagcttcgtggcggcgcaggctaaggaggaaacgtcctccacccccgctggcttcgcggcgccgtactcctcagtatccgaggacatggcaccgttcgcgtccctggacgccgctgggatgttcgagatcggcgaggacgcgtcggccgcggcggcgtactggaacgccgggagctgctggacggacgtgccggacccgagcatgatgatgtacctgctaccctag

SEQ ID NO: 66 - protein, 253 aa, Zea mays
MQEFQSIPGLAGRLFGGAAAAGLRRAQGQCGGGGASAAAAVKCPRCESTNTKFCYYNNYNLSQPRHFCKGCRRYWTKGGVLRNVPVGGGCRKAKRSSAPSTPTPTPTSAVGDAKSPRRASASSPRGSISSGSGSASPTPNGFAFSSTADVVAPPPAPIFADQAAALASLFAPPPPLPAFSFVAAQAKEETSSTPAGFAAPYSSVSEDMAPFASLDAAGMFEIGEDASAAAAYWNAGSCWTDVPDPSMMMYLLP

SEQ ID NO: 67 - DNA, 606 bp, Zea mays
gcctcttccctgctgcatctgcacgccgcccacacatgctatttttgttcactgttattgcttcttgctgtggttgttaaaccgacttggtcctgataatcctccgcgttctactgatgtgctgtcgctgcaccatgcgcgtgttgtcgtcagagcctagggctggtgaagcccatggaggagatgctgatggcggccagcgcgggcgctgcaaatccgagccaaggctcgaatccgaacccgccgccggcggcgcccgtaacgggagcgggtagcaccgagcggcgcgcgcggccgcagaaggagaagacgctcacctgcccgcggtgcaactccaccaacaccaaattctgctactacaacaactacagcctccagcagccacgctacttctgcaagacgtgccgccgctactggacggagggcggatccctccgcagcgtccccgtgggcggcggctcccgcaagaacaagcgctcctcctcctcctcctcgtcgtcggcggcggcgtccgcctccaccttctcctcggccacgagctcgtccatggccagcacaccgggggcggcgtccaagagtccggagctggcgcacgacctcaaccta

SEQ ID NO: 68 - DNA, 456 bp, Zea mays
cagagcctagggctggtgaagcccatggaggagatgctgatggcggccagcgcgggcgctgcaaatccgagccaaggctcgaatccgaacccgccgccggcggcgcccgtaacgggagcgggtagcaccgagcggcgcgcgcggccgcagaaggagaagacgctcacctgcccgcggtgcaactccaccaacaccaaattctgctactacaacaactacagcctccagcagccacgctacttctgcaagacgtgccgccgctactggacggagggcggatccctccgcagcgtccccgtgggcggcggctcccgcaagaacaagcgctcctcctcctcctcctcgtcgtcggcggcggcgtccgcctccaccttctcctcggccacgagctcgtccatggccagcacaccgggggcggcgtccaagagtccggagctggcgcacgacctcaaccta

SEQ ID NO: 69 - protein, 152 aa, Zea mays
QSLGLVKPMEEMLMAASAGAANPSQGSNPNPPPAAPVTGAGSTERRARPQKEKTLTCPRCNSTNTKFCYYNNYSLQQPRYFCKTCRRYWTEGGSLRSVPVGGGSRKNKRSSSSSSSSAAASASTFSSATSSSMASTPGAASKSPELAHDLNL

SEQ ID NO: 70 - DNA, 1158 bp, Zea mays
cccccgctactggacacacggcgggaccctccgcatttccccagacggcggcggctgccgcaggaacaagcgcgcctccagctcctcgtccccgattccgggcccctccagcaccgccgccaccagcgccgcgatggagaagaccgtcagcacgcggctgatgctgatggcgaccatcaccatggcgatgccctccccgacggcaggcctgtttgttcccgatgacatgtccccggcattcacgccgacgacgttttttatcggcttcgacgacctcgccggcatggacgagcagtaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgatgacggtggttatgcagcaggaccagccgccggagtgcagcagcagcagattaatggtggtgatcaccagaagcaggacgagaacaaagaggcggggaacggcaaaggcaacgacgacgacggcggcgggtcgtcgtcggtgtacagcttctggatgaacaccagcagcagcgacggggcacaggggtagtgcgccactgccagtgccagccacaggaggaggcgaaagtgctgctgcgaactccctcgcatcatcatctgctggtcggtgcaagtgcagcagacatccttcgtcgacgcaatcaaatcatcaaaaggtggcaacagggagtgaaagggagaagaagttcatccagcgctagttcagtctcaattagaggatagggtgcatgttcatgtttgatttatctgcattgtggtcggtcgtggttgcaagtcatcttgcaccacccttttgttttggttcagtttgcatgcgttcgttcgtcagagttttacttgcagcagtctttggatcgccggcagagattagttaggtatctatgctttgttttcgtcagttcgttctggatatgtcttaatattcctcagttt

SEQ ID NO: 71 - DNA, 753 bp, Zea mays
ccccgctactggacacacggcgggaccctccgcatttccccagacggcggcggctgccgcaggaacaagcgcgcctccagctcctcgtccccgattccgggcccctccagcaccgccgccaccagcgccgcgatggagaagaccgtcagcacgcggctgatgctgatggcgaccatcaccatggcgatgccctccccgacggcaggcctgtttgttcccgatgacatgtccccggcattcacgccgacgacgttttttatcggcttcgacgacctcgccggcatggacgagcagtaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgatgacggtggttatgcagcaggaccagccgccggagtgcagcagcagcagattaatggtggtgatcaccagaagcaggacgagaacaaagaggcggggaacggcaaaggcaacgacgacgacggcggcgggtcgtcgtcggtgtacagcttctggatgaacaccagcagcagcgacggggcacaggggtag

SEQ ID NO: 72 - protein, 250 aa, Zea mays
PRYWTHGGTLRISPDGGGCRRNKRASSSSSPIPGPSSTAATSAAMEKTVSTRLMLMATITMAMPSPTAGLFVPDDMSPAFTPTTFFIGFDDLAGMDEQYQQGFLPFSPLSLSDQAPELAPGGGGDTTPSFLDMLTGGYLDGGGYGGMSGGSDAMDMPFSLPEMGPPTTDPMPFQLQWTSSELDNYINDDGGYAAGPAAGVQQQQINGGDHQKQDENKEAGNGKGNDDDGGGSSSVYSFWMNTSSSDGAQG

SEQ ID NO: 73 - DNA, 163 bp, Zea mays
cctgcgtgcacaacaggatccttcgccttgctactgttcggcgcttccactgcacaacagtattttctacagctaggaaggagccagagcgcccatggacatgacctccaacaccagcaacagcgctgcagcagcatcatcggctccccacaaccaccagccg

SEQ ID NO: 74 - DNA, 69 bp, Zea mays
atggacatgacctccaacaccagcaacagcgctgcagcagcatcatcggctccccacaaccaccagccg

SEQ ID NO: 75 - protein, 23 aa, Zea mays
MDMTSNTSNSAAAASSAPHNHQP

SEQ ID NO: 76 - DNA, 600 bp, Zea mays
gctccagtgggtgtcatcagagcctgacaactacaccaacgacgacggtggttatgcagcaggaccagcagctggagtgcagcatcagcagcaggttgttggcgatcagcagcagcagcagcaggagaacaaagcgaatcagcgcagcaaaaacaacaacggcggcggcggcggtgggtcgtcggtgtacagctactactggaccaacaccagcaacagcgatggggcagaggggtagtgtgtgcctctgccagtgccagagccacaggagacgaaatcgctgctgcaaactccctcgcgacaaagaaggggacgcgattcaccaccgttaagcactagctcagtttgtagaggataggatgcatattcgcgtttgattaatcagcgttgtggtcgtgatggcaagtcatcacgcacatcgccctttttgttcaaatgcatgcgttagttcgttttcatcagagtttacttgctgcagtctatcatcgccggcagagattagttaggtgtctatgcttttatattcatcagtagttctggataataatgtcttcgtattcctcagtttctttgttaagtcgtctaaaactttccatgtgt

SEQ ID NO: 77 - DNA, 238 bp, Zea mays
gctccagtgggtgtcatcagagcctgacaactacaccaacgacgacggtggttatgcagcaggaccagcagctggagtgcagcatcagcagcaggttgttggcgatcagcagcagcagcagcaggagaacaaagcgaatcagcgcagcaaaaacaacaacggcggcggcggcggtgggtcgtcggtgtacagctactactggaccaacaccagcaacagcgatggggcagaggggtag

SEQ ID NO: 78 - protein, 78 aa, Zea mays
LQWVSSEPDNYTNDDGGYAAGPAAGVQHQQQVVGDQQQQQQENKANQRSKNNNGGGGGGSSVYSYYWTNTSNSDGAEG

SEQ ID NO: 79 - DNA, 126 bp, Zea mays
ggcgagccgctgccgtgcccscggtgcggcagccgggagaccaagttctgctacttcaacaactacaacgtgcggcagccgcgccacctctgccgcgcctgccgccgctactggacggccggcggt

SEQ ID NO: 80 - protein, 42 aa, Zea mays
GEPLPCPRCGSRETKFCYFNNYNVRQPRHLCRACRRYWTAGG

SEQ ID NO: 81 - DNA, 782 bp, Zea mays
atgcaacaaacgtggtctcgtgcttttcagggcatcgtcgtccccgaggagcctgggatgggcggatcgtcgtcctcaccgtcagcggaactgatcgcatgccctaggccgcccatgcacgccgcggcggcggatcaccggcgtctgcgcccgcagcacgaccagccgctcaagtgcccccggtgcgagtcgacgcacaccaagttctgctactacaacaactacagcctctcgcagccgcgctacttctgcaagacgtgccgccgctactggaccaagggcggctcgctccgcaacgtcccggtcggtgggggatgccgcaagaacaagcgagccagcgccaagaagcatcccgccgcgacgccgatgcagccacggcacgtggcggagacgggcctccacctgtccttctccggcgtgcagctgccgccgccgccgtcccccgccgccgcggccgacccgctctgcagcctcggccttctggactggaagtacgacccgatcctcgcgggttccggtggcgccgccgctggctcgttggatggcgcgagctccgaggcgcacttcgctggggcgggcatgctgggcatcccaggcggcggcgagtgctacgcgctgagttcgctccggtacgcggccgacctgggcgagcacctgcagctgcaggggctcccgttcgggctacgtgctgagcaggaatcgatggaggtgaaggcggcggcgacggagaggacgctgtcgctggactggtacagcgagacaagccgcgcgccggagagcgc

SEQ ID NO: 82 - protein, 260 aa, Zea mays
MQQTWSRAFQGIVVPEEPGMGGSSSSPSAELIACPRPPMHAAAADHRRLRPQHDQPLKCPRCESTHTKFCYYNNYSLSQPRYFCKTCRRYWTKGGSLRNVPVGGGCRKNKRASAKKHPAATPMQPRHVAETGLHLSFSGVQLPPPPSPAAAADPLCSLGLLDWKYDPILAGSGGAAAGSLDGASSEAHFAGAGMLGIPGGGECYALSSLRYAADLGEHLQLQGLPFGLRAEQESMEVKAAATERTLSLDWYSETSRAPES

SEQ ID NO: 83 - DNA, 2451 bp, Zea mays
cccccgtgccttcgcttttccgcaacagattgctcccggtccactggcttggcgtcccaaccccacatacacgcgcgcaacagttgccagtgcatttataaaaaccgggagattctcctcgaaacacaagctccagcaaatgcaaatcacacggcaagtgactcgatcatcacacgagtcgaacggagccgtaacctttttcattcttcccccgcaggtggccgacccgcctcatcactgttgctagtatccgtccgtccatgcatctgcggctcccacctgtcttgacatcgtgacggatggatgggccaagccgaggtcgagggcttttaaagtagcaacgaccgacccccttcgtccccgcccccctagctagctctctccctgtctgtcaccgccgagtctctcgcagtctgcctctcgctctcgctacggagtccggaccactctctctctctctctctctctctctctctctctctctctctctctctctctctctctctctttctagcagtctagtagctgcgactagcacttccacctactggtgttggtgtttgcttgccttgacgaacgaaggagttggttcgttgcgttccaccttccaaatgctgtcccacgtcgagatggcccccggcgggttcaagctgttcggcaaggtcatcacgcagtgcgccgagagcgccccgccggcgccgccggaggctgctgccgcggcgtcgagggccacggccttcgcgcgggagagggacgacccggacgagcgcgaccagccgatggtgaagcgggaggcggcggcgtccgaccacgacttcgccgccgtggacaagcaccaccagcactccggcggcagcggcgggccgggaccgggaccggacgagagcgacgacagcaaagggcagcagcagcagcagcaccacccgcgcccgcggcatcatcagcatcaccatcagcagcagcaggacaccgtggaggcgcgcgccgcggcggccgcgtcctcggcgccgccgctgccgtgcccgcggtgccggagccggaacaccaagttctgctacttcaacaactacaacgtcaaccagccgcgccacttctgcaaggactgccaccgctactggacggcgggcggcgcgctgcgcaacgtccccgtgggcgccggccgacgcaagaaccggccgctcggccccgtcgtcgccggagccgtgcccgtgcccgtgcccgtgccggcgcaccaccaccaccaccacctgcaccccgctgctgctgctgctgcggcggcggcgggcttcgtcctcgggttccccggccagcagcacccttcgtcgccgacgtcgccgtcgccgtcgccggtctacgccgaccggtggcctgtatgtccggaccggcggttctgagttctggttagggtggtttgacttgcgtaatttgctggtgatgaggcggggaaggctgccttgtctgtctgtacgtgggtgtgcgtgcgtgagaggctggttgatggaggacgccggcatggaaatggcatctgtttccttctcttttctctgcctcgattttcttgtttcccgctaaattagctcggtgtagctgccagtttaacctcctttgcagacgtgtgtaaaaagcgtactgtaatgtaacagcagaccagggacaggtcaaaaactcgaggtcagcgtgcgtgctcaactagtgccgacgaaggtgatgcgatcccatgctgtccagtccagtccttagctcgcatcccgggagtcgttggtgatcatcgcacgaccaaccccgcgttgtacattccttcctgggaaaaggtgacgacggacggagtgctgatgctctgcctctgcgcgcagatggagctcacggcattggcttcgcagcgctagaagcctgcccactgcccagcacgaatgagagagagaatgtgtgtgctctgagagtgagagtagaatggaggaaagacccgtaccatgtcgatgtcgatgtggtgttgaaagccaaaaaggcaggcgatgatgcggttgaccgcgcgcgcaggcgcagaggacacagcctttgctacatggccacggtcacattttccgtgtgtggttggcccagcccaacgcttttcaccacccttttggaaagtttcatcacgtcacgatagggcacctggctctggctggccgccgggcgccttttattttaccggccatcaatgcccgccccccgcccggcgaaagatcgcgcggcctttactaaaaaagcgccgggagataaatattccaaccgtcaacttgtcaggagtctgggtgggaggaagaggaacggaaccaagagaagactactcctactagctgctgccgctttgcgggctttaaattgctcgcaaagtttactttagtacgcgggagcggccgcgagacccgtctctcggctctcgcccgcgtcgtcgcgtgtggttggttg

SEQ ID NO: 84 - DNA, 570 bp, Zea mays
atgctgtcccacgtcgagatggcccccggcgggttcaagctgttcggcaaggtcatcacgcagtgcgccgagagcgccccgccggcgccgccggaggctgctgccgcggcgtcgagggccacggccttcgcgcgggagagggacgacccggacgagcgcgaccagccgatggtgaagcgggaggcggcggcgtccgaccacgacttcgccgccgtggacaagcaccaccagcactccggcggcagcggcgggccgggaccgggaccggacgagagcgacgacagcaaagggcagcagcagcagcagcaccacccgcgcccgcggcatcatcagcatcaccatcagcagcagcaggacaccgtggaggcgcgcgccgcggcggccgcgtcctcggcgccgccgctgccgtgcccgcggtgccggagccggaacaccaagttctgctacttcaacaactacaacgtcaaccagccgcgccacttctgcaaggactgccaccgctactggacggcgggcggcgcgctgcgcaacgtccccgtgggcgccggccgacgcaagaaccggccgctc

SEQ ID NO: 85 - protein, 190 aa, Zea mays
MLSHVEMAPGGFKLFGKVITQCAESAPPAPPEAAAAASRATAFARERDDPDERDQPMVKREAAASDHDFAAVDKHHQHSGGSGGPGPGPDESDDSKGQQQQQHHPRPRHHQHHHQQQQDTVEARAAAAASSAPPLPCPRCRSRNTKFCYFNNYNVNQPRHFCKDCHRYWTAGGALRNVPVGAGRRKNRPL

SEQ ID NO: 86 - DNA, 1900 bp, Zea mays
ttttgccggcctcctcgtctcctccggttccggctacctacctgcgggacgtcgacctcgtccgtctgtcgcctagctagctgcctcggttttcgcttgaagcagcagcagcagcaagccgggagcttctgtgtttgtttcgatcggagtgagaagaagatggttgcggagtgccaaggaggaggagacttcctcatcaagctcttcgggaagaccatccccgtgccggagccggagccggagtccggcgacgccaagatcgtggacgccgatgacgaccccaagaagcacagcgacggcgacgctgccaggcagagggagaagctgagggagcccgacaaggtgctgccgtgcccgcgctgtaacagcgcggacaccaagttctgctacttcaacaactacaacgcgaagcagccgcgccacttctgcaagcgctgccagcgctactggaccgcgggcggcgccatgcgcaacgtgccagtgggggccggccgccgcaagaacaagaacgccgccgcctcgcactcgcacctcctccataggacgacgacgactgccaacggcgcgatgctcagcttcgcccctcccggtcctggccttgcctgcctctgcctggacctcgccgcgcagttcggccacctggccccggtcagggacgccgccgggaccccgtctcgtccttgcacttgcagcgaagggtcgaccgacagggacggcacgtcttccgtcgtagacgaatctgcagcaagcggggacggaccagtgcagctgcaccaccacccagcaagcgttaacaccgggtggccgccatacagcagcccacctccggcggcgccgtatttctcgccgggcatctcgattccggtataccccgccgcgccggggtactggggctgcatggttcccggggcttggagcctgccatggccggtgcagccgcagcccccgtcgtcgcggtcgcaggggcagggcctctcgtcgtcgtcgtcgccgccgccgcccaccaccaccaccatcggtactccttcagtctcatcatcatccggggccgctgactttgactcccacgccctggggctgggcctgggcaagcacccgcgggaccgggacggcgacgacgggaggaagaggaacggcagtgcccatggcagcggcaacgccaaggtgtgggctcccaagaccatccggatagacgatgtggacgaggtggccaggagctccatctggtccctcgtcggggtcagaggcgacagggagcagcagggcgcagccgcgcaggtgacaagctcgggacagtgttcgagcccaggggcctgcaggccaccaagaaggccatggcgacaagctcgccgctccttcacgccaaccccgtcgcgctcacgcgctcggtggcgttccatgagggctcttgattttgccatctcaacatgatgctcctaatcagactgacagcactgatttgttttggataaatcatcagaggacccctcacatgcacgctgattcacatacctctttggttgcgtaggagaatcagactcctcgtatgcattgaccataactcgactcatgtttagtacgctgcatcttaaaaactgagcacacatacactggtgcaacctaatgttatttaggtgcacctgtagatacccagaaaacttacgtattcgggcggccaggctcacacgagcctcgcatgcaagcagagatccaatcacactggtagctacttgtggacggcggggcggcacgcaatttggatgggagaaagaacaggagatcaataagaggttcctgctcgtagataaataaataacattatctaactttttcgttgcgttctctgtgcaagtcctttttttcctcgtgcccttttcactcttttgtttgggacaaattg

SEQ ID NO: 87 - DNA, 1272 bp, Zea mays
atggttgcggagtgccaaggaggaggagacttcctcatcaagctcttcgggaagaccatccccgtgccggagccggagccggagtccggcgacgccaagatcgtggacgccgatgacgaccccaagaagcacagcgacggcgacgctgccaggcagagggagaagctgagggagcccgacaaggtgctgccgtgcccgcgctgtaacagcgcggacaccaagttctgctacttcaacaactacaacgcgaagcagccgcgccacttctgcaagcgctgccagcgctactggaccgcgggcggcgccatgcgcaacgtgccagtgggggccggccgccgcaagaacaagaacgccgccgcctcgcactcgcacctcctccataggacgacgacgactgccaacggcgcgatgctcagcttcgcccctcccggtcctggccttgcctgcctctgcctggacctcgccgcgcagttcggccacctggccccggtcagggacgccgccgggaccccgtctcgtccttgcacttgcagcgaagggtcgaccgacagggacggcacgtcttccgtcgtagacgaatctgcagcaagcggggacggaccagtgcagctgcaccaccacccagcaagcgttaacaccgggtggccgccatacagcagcccacctccggcggcgccgtatttctcgccgggcatctcgattccggtataccccgccgcgccggggtactggggctgcatggttcccggggcttggagcctgccatggccggtgcagccgcagcccccgtcgtcgcggtcgcaggggcagggcctctcgtcgtcgtcgtcgccgccgccgcccaccaccaccaccatcggtactccttcagtctcatcatcatccggggccgctgactttgactcccacgccctggggctgggcctgggcaagcacccgcgggaccgggacggcgacgacgggaggaagaggaacggcagtgcccatggcagcggcaacgccaaggtgtgggctcccaagaccatccggatagacgatgtggacgaggtggccaggagctccatctggtccctcgtcggggtcagaggcgacagggagcagcagggcgcagccgcgcaggtgacaagctcgggacagtgttcgagcccaggggcctgcaggccaccaagaaggccatggcgacaagctcgccgctccttcacgccaaccccgtcgcgctcacgcgctcggtggcgttccatgagggctcttgattttgccatctcaacatga

SEQ ID NO: 88 - protein, 423 aa, Zea mays
MVAECQGGGDFLIKLFGKTIPVPEPEPESGDAKIVDADDDPKKHSDGDAARQREKLREPDKVLPCPRCNSADTKFCYFNNYNAKQPRHFCKRCQRYWTAGGAMRNVPVGAGRRKNKNAAASHSHLLHRTTTTANGAMLSFAPPGPGLACLCLDLAAQFGHLAPVRDAAGTPSRPCTCSEGSTDRDGTSSVVDESAASGDGPVQLHHHPASVNTGWPPYSSPPPAAPYFSPGISIPVYPAAPGYWGCMVPGAWSLPWPVQPQPPSSRSQGQGLSSSSSPPPPTTTTIGTPSVSSSSGAADFDSHALGLGLGKHPRDRDGDDGRKRNGSAHGSGNAKVWAPKTIRIDDVDEVARSSIWSLVGVRGDREQQGAAAQVTSSGQCSSPGACRPPRRPWRQARRSFTPTPSRSRARWRSMRALDFAIST

SEQ ID NO: 89 - DNA, 1496 bp, Zea mays
gggctagggctagggaagcccatggaggagatgctcatggctggaaacgcaaatctaaaccagaatccaaacccaccgccggctgcgccctcggctcccggcgcccagagggcaggcgctcccgccgcggttgcggcagcgccgcctagcgctggcgcgaccggaggagcggggccggagcggcgggcgcggccgcagaaggagaaggcgctcaactgcccgcggtgcaactccaccaataccaagttctgctattacaacaactatagcctccagcagccgcgctacttctgcaagacgtgccgtcgctactggaccgagggcggctcgctccgcaacgttccggtgggcggcggctccaggaagaacaagcgctcgtcctcggccgtgtcgtcggcggctgcggccgccgccgcctccacctccgcggctatgtctggcacggtccccgtggggctcgcggccaagaaccccaagttgatgcacgagggcgcgcacgacctcaacctggcgttccctcaccacaacggccgcgctctgcagccaccggagttcccggcgttcccgagcctggagagcagcagcgtgtgcaaccccggagctgccatgctgggcaacggcgccgccggcaggggcatgggcgcgctctcgggtttggagctgctgaggagcacgggctgctacgtcccactgcagcactttcagctagggatgcccgcggagtacgcagccgcgggattctcgctgggcgagttccgcgttccgccgccgccgcagtctcagagtgtgttcgggttctccctggacacgcacggcacggggggagttggcggcgccgggggttacagtgccgggctgcaggagagcgcggctggcaggatgctcttccccttcgaggacttgaagccggcagtgagcgctgctggcggcggcgcgagcaacggagctgatcatcatcactacgagcacagcaaagatcaagcagcaggcgacggcggcggagccagcggcgtcaccggcggccacgaggctccagcaggattctggagcaatagcatgatcgggaatggcagcagcaatggtggcggaggctcttggtaaatggtaaggcggcgcgccatgcatggctcatgcacgcgccgttgtcagcatgcggagataggggggtagagacaaggatcggagttaataagagggtggaggtgattagtgttcacatatggcgtttgatctctctggttgtttgccttctctttttattttcggttcttggtgttcgtgtgtgtcaatcgacttgggggctaacacagcaggatatacagattgttaaggcggtgaaccgattatttacagcttctcttagccaatccatctctcgttttctgattctgtaatccaaggaacaagctgtcaatcacgttgtgcattgcatgcgcattatgtagagtatgtattaattgagcagctctagctatgttagtgttacaagttaaatc

SEQ ID NO: 90 - DNA, 1101 bp, Zea mays
gggctagggctagggaagcccatggaggagatgctcatggctggaaacgcaaatctaaaccagaatccaaacccaccgccggctgcgccctcggctcccggcgcccagagggcaggcgctcccgccgcggttgcggcagcgccgcctagcgctggcgcgaccggaggagcggggccggagcggcgggcgcggccgcagaaggagaaggcgctcaactgcccgcggtgcaactccaccaataccaagttctgctattacaacaactatagcctccagcagccgcgctacttctgcaagacgtgccgtcgctactggaccgagggcggctcgctccgcaacgttccggtgggcggcggctccaggaagaacaagcgctcgtcctcggccgtgtcgtcggcggctgcggccgccgccgcctccacctccgcggctatgtctggcacggtccccgtggggctcgcggccaagaaccccaagttgatgcacgagggcgcgcacgacctcaacctggcgttccctcaccacaacggccgcgctctgcagccaccggagttcccggcgttcccgagcctggagagcagcagcgtgtgcaaccccggagctgccatgctgggcaacggcgccgccggcaggggcatgggcgcgctctcgggtttggagctgctgaggagcacgggctgctacgtcccactgcagcactttcagctagggatgcccgcggagtacgcagccgcgggattctcgctgggcgagttccgcgttccgccgccgccgcagtctcagagtgtgttcgggttctccctggacacgcacggcacggggggagttggcggcgccgggggttacagtgccgggctgcaggagagcgcggctggcaggatgctcttccccttcgaggacttgaagccggcagtgagcgctgctggcggcggcgcgagcaacggagctgatcatcatcactacgagcacagcaaagatcaagcagcaggcgacggcggcggagccagcggcgtcaccggcggccacgaggctccagcaggattctggagcaatagcatgatcgggaatggcagcagcaatggtggcggaggctcttggtaa

SEQ ID NO: 91 - protein, 366 aa, Zea mays
GLGLGKPMEEMLMAGNANLNQNPNPPPAAPSAPGAQRAGAPAAVAAAPPSAGATGGAGPERRARPQKEKALNCPRCNSTNTKFCYYNNYSLQQPRYFCKTCRRYWTEGGSLRNVPVGGGSRKNKRSSSAVSSAAAAAAASTSAAMSGTVPVGLAAKNPKLMHEGAHDLNLAFPHHNGRALQPPEFPAFPSLESSSVCNPGAAMLGNGAAGRGMGALSGLELLRSTGCYVPLQHFQLGMPAEYAAAGFSLGEFRVPPPPQSQSVFGFSLDTHGTGGVGGAGGYSAGLQESAAGRMLFPFEDLKPAVSAAGGGASNGADHHHYEHSKDQAAGDGGGASGVTGGHEAPAGFWSNSMIGNGSSNGGGGSW

SEQ ID NO: 92 - DNA, 2028 bp, Zea mays
taatttaattttagtaactaaacaagagcatacaatatatctatgtgtgggaggtgttcttatgctgttatttttgcaagctgataccagatgctacctagggttataacagcacgatgagaacattcttcagacctagctagggttacacatgatgaccggcatctaaggacacaagaatttatgtgtcctcaggttctatctactagtacgagatcataggaaatccatgcacatatgggacattaatgctttaagaaatcaagacaagtacaccttgcatgtctctcatgatatcaaaggttcgtcgttgtaactttaacatgtgttcaaaagcgcaactacgcgtagaggagatcatgcatggggccggtctacaactgatccatgtgattccaaaagctttggataacataggcggatcaagctgatcagagatctaaaatctgatctaattgatgcctccatcatgaaatatcataatagcctttggagctttgcatgcagctcgtactttagcctactgtactgtatatgcatgctgaccatgtggtcctttcccgcattccgaggcttccattctgtcacagataatgatttgcgatcacactgccataacgtggtcttgtgcttttcagggcatcgttcccgaggagcctgggatgggatcgtcgtcaccgtcagcggagctgatcgcgtgccctaggccaatgcacgccacggctgccgcggcggaccggcgtctgcgcccgcagcacgaccagccactcaagtgcccgcgatgcgagtccacgcacaccaagttctgctactacaacaactacagtctctcccagccgcgctacttctgcaagacgtgccgccgctactggaccaaaggcggatcgctccgcaacgtcccggttggtgggggatgccgcaagaacaagcgcgccagcgccaagaaaccacccgcgccaccgatgcagccgccgcacatgtcggagacgggcctccacctgtccttctccggcgtgcagcagctgccgccggccgatccgctttgcagcctcggccttttggactggaagtatgacccgatcctcacaggttccggtggcgtcgctggctcgttggatggcgccagctccgagacgcacttcgcagggacgggcatgctgggcatcccaggtggcagcgggggcggcgagtgccacgcgctgagcgcgctccggtacgcggccggcctgggcgagaacctgcaggtgcagggtctcccgtttggggcgcgcgctgagcacgacgcgatggagatgaagcctgcggcgacggacaggctgctctcgctggactggtacagcgagacaagccgcgcgccagagagcgccatcagctcgttgggcgcgctgggcctgtggagcggcatgctcggcggtgcgcaccagcaccatggctcgtcggcggccatctaagaccctagcaaaacgtgcctccatgcttgcgttgcattgcaccagtttgctaatggcgttaacaagtatgagtgactacgtagaaatgcttgtttttttacctgtttttatcttctgtttcgtttcgtcgtttgttttttggacgatccatgtttgccgctcgtcatttcagatgatgtctctgttgttgtaccgcttcaaagttcaaacacttgctgtggttcaaattacagtttgctggtgattgatgagcccaggttttatacgtatggaaatatggattatgtatggaactatgtatctcttctctactcctataaagtatattaattacctagactccactgttgtctcttttatatataaaaaagctcttatacatctttctaatctaactaaaccgttagatcgtaactagtcggttgaccgtatgttacgacagctcacaataatacccacataaattatctacaaaaaaagatctcaatattttttatttattgtctctgctcttatcataaagtcataatgtgttttatgaacgcactggtacaataggcaacaaatcagagacttc

SEQ ID NO: 93 - DNA, 858 bp, Zea mays
atgatttgcgatcacactgccataacgtggtcttgtgcttttcagggcatcgttcccgaggagcctgggatgggatcgtcgtcaccgtcagcggagctgatcgcgtgccctaggccaatgcacgccacggctgccgcggcggaccggcgtctgcgcccgcagcacgaccagccactcaagtgcccgcgatgcgagtccacgcacaccaagttctgctactacaacaactacagtctctcccagccgcgctacttctgcaagacgtgccgccgctactggaccaaaggcggatcgctccgcaacgtcccggttggtgggggatgccgcaagaacaagcgcgccagcgccaagaaaccacccgcgccaccgatgcagccgccgcacatgtcggagacgggcctccacctgtccttctccggcgtgcagcagctgccgccggccgatccgctttgcagcctcggccttttggactggaagtatgacccgatcctcacaggttccggtggcgtcgctggctcgttggatggcgccagctccgagacgcacttcgcagggacgggcatgctgggcatcccaggtggcagcgggggcggcgagtgccacgcgctgagcgcgctccggtacgcggccggcctgggcgagaacctgcaggtgcagggtctcccgtttggggcgcgcgctgagcacgacgcgatggagatgaagcctgcggcgacggacaggctgctctcgctggactggtacagcgagacaagccgcgcgccagagagcgccatcagctcgttgggcgcgctgggcctgtggagcggcatgctcggcggtgcgcaccagcaccatggctcgtcggcggccatctaa

SEQ ID NO: 94 - protein, 285 aa, Zea mays
MICDHTAITWSCAFQGIVPEEPGMGSSSPSAELIACPRPMHATAAAADRRLRPQHDQPLKCPRCESTHTKFCYYNNYSLSQPRYFCKTCRRYWTKGGSLRNVPVGGGCRKNKRASAKKPPAPPMQPPHMSETGLHLSFSGVQQLPPADPLCSLGLLDWKYDPILTGSGGVAGSLDGASSETHFAGTGMLGIPGGSGGGECHALSALRYAAGLGENLQVQGLPFGARAEHDAMEMKPAATDRLLSLDWYSETSRAPESAISSLGALGLWSGMLGGAHQHHGSSAAI

SEQ ID NO: 95 - DNA, 1988 bp, Zea mays
taaagaaaccaaggttgacacgccacaacaggagaagggtaatgaaatggaggttgatgcgccacaaaaggaacatgacgacgaaatgaaaattgacgcacaacaagaggaaaaagatgaacaaatggaagccgtcgcatcaccaacgcatggatatatagaaccagccaatttacctccctcggagtcggagcacaagaaaggagacgagggaccgatggacagtgccgaagataaagcagcatcagatgcaaaggggcagaacgagaagacatcaaatgaggaatcaggccaggacagggcacttaataagaagccagataagatcctaccttgccctcggtgcaacagcatggatacaaagttctgctattacaacaactacaacgttaatcaaccaaggcacttctgcaagaactgccagcggtactggactgcagggggcaccatgaggaacgtacctgttggcgccgggcggcgcaagagtaagaacgcgtcattgcactaccgtcaactactgatggcccctgactgtatgctggggcctagagtggacatatcaaagccagtgctccctgaaggtcttgcatcatctccacctgccccgacacagccagccagtagaaatggaacggtcctaaaatttgggcatgaggtaccattttgcgagtcaatggtgtcggcgctgaacattgatgagcagaacggcaacagccctggaggaccaacagcaagaggtgaaaacagggaagataataataaccctggatccggcacaccaccatacaacggtgtgcctgaaaccatggcccccgtcgtcggcaagaacggggcaccagttcattgtaacggggttgccccagtgcctcagtattaccttggaacccctttcatgtacccttggaatgtaggatggagcaacgtgcctgtgatggtgccaggtaaaagcatgcccgaacctgctcctgctccagagagctgcagtactagctcagctgtatggatgaaccctcccatgatgccaggctcaagacctcctcctagtccagcgtttccataccctctcgtgccacccgggctctggggctgtttttccggatggccagccacggcttggaacgtaccatggaccagaaccaacgtctgcgtgtcgccgccgccgccgccgccgctgtcgccgtcgccatcaagcaacagcagcagctgctcgggcaacgggtctcctactctgggcaagcattccagggacaccaatccgctgagagaggagaaaagagagaagtcgctgtgggttcccaagacgctccggatcgatgaccctgatgacgccgccaagagttcgatatgggccaccctcggcatcaagcccggagaccctggcactttcatcaagcctttccagtccaaggtcgagagcaagggccagaggtcggacgctgctcaggtcttgcaggcgaacccggcggcgatgtcgcgctcacagtcgttccaggagagctcttgacttcatacgcaaagtctgccttatatatagttttggtactgtaatccttacatgctggtagagttttgtggcatgcgaaaagtgacttcgtacctgaagcctatcttattgtttgtttcggtactgtattccttactacatgctagtagagctgtgcggtatgtggaaaggacaacagagagcttcttgtatagccaattagccacctagtggaacttgcaaagagcttccatgagaactctcgagatacttatgtattgtaaatacttgacatactggtgttgtatatacttgacttactggtgttgcaaaggaacttaaaaaggagggactaaaaatttaggtgctgtttgaaatgtaatgcaaatggtaatggtttacacttgattactggcgataataagtttgaatatattggtatcaatttttagtgtgttatctgattacgattgaactaaaataaacattatttaacgttatcagt

SEQ ID NO: 96 - DNA, 1458 bp, Zea mays
atggaggttgatgcgccacaaaaggaacatgacgacgaaatgaaaattgacgcacaacaagaggaaaaagatgaacaaatggaagccgtcgcatcaccaacgcatggatatatagaaccagccaatttacctccctcggagtcggagcacaagaaaggagacgagggaccgatggacagtgccgaagataaagcagcatcagatgcaaaggggcagaacgagaagacatcaaatgaggaatcaggccaggacagggcacttaataagaagccagataagatcctaccttgccctcggtgcaacagcatggatacaaagttctgctattacaacaactacaacgttaatcaaccaaggcacttctgcaagaactgccagcggtactggactgcagggggcaccatgaggaacgtacctgttggcgccgggcggcgcaagagtaagaacgcgtcattgcactaccgtcaactactgatggcccctgactgtatgctggggcctagagtggacatatcaaagccagtgctccctgaaggtcttgcatcatctccacctgccccgacacagccagccagtagaaatggaacggtcctaaaatttgggcatgaggtaccattttgcgagtcaatggtgtcggcgctgaacattgatgagcagaacggcaacagccctggaggaccaacagcaagaggtgaaaacagggaagataataataaccctggatccggcacaccaccatacaacggtgtgcctgaaaccatggcccccgtcgtcggcaagaacggggcaccagttcattgtaacggggttgccccagtgcctcagtattaccttggaacccctttcatgtacccttggaatgtaggatggagcaacgtgcctgtgatggtgccaggtaaaagcatgcccgaacctgctcctgctccagagagctgcagtactagctcagctgtatggatgaaccctcccatgatgccaggctcaagacctcctcctagtccagcgtttccataccctctcgtgccacccgggctctggggctgtttttccggatggccagccacggcttggaacgtaccatggaccagaaccaacgtctgcgtgtcgccgccgccgccgccgccgctgtcgccgtcgccatcaagcaacagcagcagctgctcgggcaacgggtctcctactctgggcaagcattccagggacaccaatccgctgagagaggagaaaagagagaagtcgctgtgggttcccaagacgctccggatcgatgaccctgatgacgccgccaagagttcgatatgggccaccctcggcatcaagcccggagaccctggcactttcatcaagcctttccagtccaaggtcgagagcaagggccagaggtcggacgctgctcaggtcttgcaggcgaacccggcggcgatgtcgcgctcacagtcgttccaggagagctcttga

SEQ ID NO: 97 - protein, 485 aa, Zea mays
MEVDAPQKEHDDEMKIDAQQEEKDEQMEAVASPTHGYIEPANLPPSESEHKKGDEGPMDSAEDKAASDAKGQNEKTSNEESGQDRALNKKPDKILPCPRCNSMDTKFCYYNNYNVNQPRHFCKNCQRYWTAGGTMRNVPVGAGRRKSKNASLHYRQLLMAPDCMLGPRVDISKPVLPEGLASSPPAPTQPASRNGTVLKFGHEVPFCESMVSALNIDEQNGNSPGGPTARGENREDNNNPGSGTPPYNGVPETMAPVVGKNGAPVHCNGVAPVPQYYLGTPFMYPWNVGWSNVPVMVPGKSMPEPAPAPESCSTSSAVWMNPPMMPGSRPPPSPAFPYPLVPPGLWGCFSGWPATAWNVPWTRTNVCVSPPPPPPLSPSPSSNSSSCSGNGSPTLGKHSRDTNPLREEKREKSLWVPKTLRIDDPDDAAKSSIWATLGIKPGDPGTFIKPFQSKVESKGQRSDAAQVLQANPAAMSRSQSFQESS

SEQ ID NO: 98 - DNA, 1788 bp, Zea mays
atagggagtatgccggagaagtggagatagtattagtctgttgctgatttacagttcgtcgagctctctttgccgagtgtcacactcggcaaagccttcgtcgaatgttttctaggctttgctgagacactcgacaaagaggacgattccggtagtgatagatggagtgggtaaaatgagctaaaattactttaggggttatatggacgtcatctacaggttgagaggagtaatttagagtttttctcaataacaaaatactaatatttaagataggagaaaggaccgtattgttatatatggtttgggtgttggacttatagagtgaagttgaaaataatgagtgatatagagaatgagatagggagtatgccggagaagtggagatagtattagtctgttgctgatttggcttagctttacagccaaatttattaattttgctttgagcaaataagacgaacgttttgttttacaatcctagcacaggcgttataggttctgtttgcaacccaagcattatttcaccttcaggatttattcgtttggatccctcccgtagtattttgcatgggatttgatccccacgatacctctaaattccgtcgtaaacaggccgtcagagttcagacacatggtccatatgcattgtttgttgatggtagcatgcaaagagcaagaatcaggaatttgctccatcagtcctctgcctgcttcgtctgcaagagaaagctacgtaacctttgtggctgtgggcctgtgccaccccccgctgctgcttcctgctgtcgtcctcgttcgtccctacctagctgcttacctctctcctgcatgctgcgttgctgccatcacctccgtgccatccgcggcgcgacccggtccgagcagtccctctctctctctctgctctgctccgctcgacaagcactgtgactctgtgagcgacactactgcacaccgccaccgtggccggccggcgtccgctgcgctatatgtatacaagcagctagcaccaagcagcacggctctccaactcacagtgccacaccggcaggcaaggcatcgtcgaccgacggcgcgccgcagcagcagcttctagctccaaaccgaaccaacacgtgctatccaccatggaggcgccgctgcaccagttccccttccccgtgccgccgccgccgcagccgcaggacgcgctgctgcagcaggcggtggcggcgcgcgcgctgatgatggccgggaaaagggcgccgctgcaggctaccgcggcggcgggacgggagcagtgcccgcggtgcgcgtcgcgggacaccaagttctgctactacaacaactacaacacggcgcagccgcggcacttctgccgcgcgtgccgccgctactggacgctggggggatccctccgcaacgtccccgtgggcgggtccacgcgcaagcgccagcgcccggcgcggcccacccgcgccatggccgccgccgccgccacggcggctacgcaaacggcaacaacgaccgcctgcagcccgttcgccgtctctccagcaacaatgctgatacagggcgcggcaggcggcggcctcctcgtcagctcgctgctgctgggctcggcgtccgcgtcaccgctgctcgcgctcggcacagcgccgctgctggaaggccggcttggcttcgacctcggcctcggggacgcagcagcgctagcagccgccgacgggactcctggcgacttggctcaccaccaccagccgctgacgctgggcgccgggccgctcccgtggcccgcagcgacga

SEQ ID NO: 99 - DNA, 775 bp, Zea mays
caagcagcacggctctccaactcacagtgccacaccggcaggcaaggcatcgtcgaccgacggcgcgccgcagcagcagcttctagctccaaaccgaaccaacacgtgctatccaccatggaggcgccgctgcaccagttccccttccccgtgccgccgccgccgcagccgcaggacgcgctgctgcagcaggcggtggcggcgcgcgcgctgatgatggccgggaaaagggcgccgctgcaggctaccgcggcggcgggacgggagcagtgcccgcggtgcgcgtcgcgggacaccaagttctgctactacaacaactacaacacggcgcagccgcggcacttctgccgcgcgtgccgccgctactggacgctggggggatccctccgcaacgtccccgtgggcgggtccacgcgcaagcgccagcgcccggcgcggcccacccgcgccatggccgccgccgccgccacggcggctacgcaaacggcaacaacgaccgcctgcagcccgttcgccgtctctccagcaacaatgctgatacagggcgcggcaggcggcggcctcctcgtcagctcgctgctgctgggctcggcgtccgcgtcaccgctgctcgcgctcggcacagcgccgctgctggaaggccggcttggcttcgacctcggcctcggggacgcagcagcgctagcagccgccgacgggactcctggcgacttggctcaccaccaccagccgctgacgctgggcgccgggccgctcccgtggcccgcagcgacga

SEQ ID NO: 100 - protein, 258 aa, Zea mays
QAARLSNSQCHTGRQGIVDRRRAAAAASSSKPNQHVLSTMEAPLHQFPFPVPPPPQPQDALLQQAVAARALMMAGKRAPLQATAAAGREQCPRCASRDTKFCYYNNYNTAQPRHFCRACRRYWTLGGSLRNVPVGGSTRKRQRPARPTRAMAAAAATAATQTATTTACSPFAVSPATMLIQGAAGGGLLVSSLLLGSASASPLLALGTAPLLEGRLGFDLGLGDAAALAAADGTPGDLAHHHQPLTLGAGPLPWPAAT

SEQ ID NO: 101 - DNA, 1292 bp, Zea mays
cagcagcaaccactccagtgtctcctcgacggcggtgctggcagcgaccaccaccacctgatgcctccgccgtcctgcctggcgccgctgcgcggaggtcctgctgacacggcggcgagcgctccagcgggaggcggctcgtccacctcggtgccgacgacggcgggagcaggggcaggggcgggggcgacgcagcctcgccccgtcgtgtcgatggcggagcgcgccaggctcgtgcgcgtgccgctcccggagcccggcacgctccgctgcccgcgctgcgactccgccaacaccaagttctgctactttaacaactactcgctctcgcagccgcgccacttctgcaaggcgtgccgccgctactggacccgcggcggcgcgctccgcaacgtgcccgtcggcggcgggtgcaggcgcaacaccaagcgctccagcaagaagtcctcccgcggtggcggcgcgggcgccacggcggccacctcctcgtcctccaccacctccacctccaccacggccaccaccaccacgacgagcgcggccatggcggcggccgaggccatcgccagcatgcaggcgcagctgccccacctcggcctcccgcccgcagcggccgccgccgcgctcgaggcctcgctggagggctaccaccaccactacctcccgctccagatgcagccgcagttcctgcagcaggctggcctgcacggctaccatttcgccgacgacggcagcggcgtcctcgcagacgggttcccgaggggcgtcgttgcctcgggccttctcgcgcagctcgccgcggtgaagatggaggagcacagcagcaacggcggaggtgccgtcacggcgcatcaacaagagcagtcgtactcgtactggcccggcagcaccggcggtggcagtgggtggccggcggagttcttgtcggggttcagctcgtcgtcgtcggggaatgtgttgtgagttgcatgtggccgtccaggtccagctggggataaatatgcatacatgcattctgacgaagctgaactagctcactctcctcagttagtttatgtggtttggaacttaattggagtcgtcttcttcaattagggctggtgatcgatcttgttttgggtcgctgcatgtttttttccttacgagttttaagctgcaggttaatgtcgtgtagttggtagtttttgcatgtagtgggaggtgcatgtgttaactcacggatgtgatacgtgtcaggttacttaactatactatgtttaattacatgagaacttggggcatagttgtactag

SEQ ID NO: 102 - DNA, 960 bp, Zea mays
cagcagcaaccactccagtgtctcctcgacggcggtgctggcagcgaccaccaccacctgatgcctccgccgtcctgcctggcgccgctgcgcggaggtcctgctgacacggcggcgagcgctccagcgggaggcggctcgtccacctcggtgccgacgacggcgggagcaggggcaggggcgggggcgacgcagcctcgccccgtcgtgtcgatggcggagcgcgccaggctcgtgcgcgtgccgctcccggagcccggcacgctccgctgcccgcgctgcgactccgccaacaccaagttctgctactttaacaactactcgctctcgcagccgcgccacttctgcaaggcgtgccgccgctactggacccgcggcggcgcgctccgcaacgtgcccgtcggcggcgggtgcaggcgcaacaccaagcgctccagcaagaagtcctcccgcggtggcggcgcgggcgccacggcggccacctcctcgtcctccaccacctccacctccaccacggccaccaccaccacgacgagcgcggccatggcggcggccgaggccatcgccagcatgcaggcgcagctgccccacctcggcctcccgcccgcagcggccgccgccgcgctcgaggcctcgctggagggctaccaccaccactacctcccgctccagatgcagccgcagttcctgcagcaggctggcctgcacggctaccatttcgccgacgacggcagcggcgtcctcgcagacgggttcccgaggggcgtcgttgcctcgggccttctcgcgcagctcgccgcggtgaagatggaggagcacagcagcaacggcggaggtgccgtcacggcgcatcaacaagagcagtcgtactcgtactggcccggcagcaccggcggtggcagtgggtggccggcggagttcttgtcggggttcagctcgtcgtcgtcggggaatgtgttg

SEQ ID NO: 103 - protein, 320 aa, Zea mays
QQQPLQCLLDGGAGSDHHHLMPPPSCLAPLRGGPADTAASAPAGGGSSTSVPTTAGAGAGAGATQPRPVVSMAERARLVRVPLPEPGTLRCPRCDSANTKFCYFNNYSLSQPRHFCKACRRYWTRGGALRNVPVGGGCRRNTKRSSKKSSRGGGAGATAATSSSSTTSTSTTATTTTTSAAMAAAEAIASMQAQLPHLGLPPAAAAAALEASLEGYHHHYLPLQMQPQFLQQAGLHGYHFADDGSGVLADGFPRGVVASGLLAQLAAVKMEEHSSNGGGAVTAHQQEQSYSYWPGSTGGGSGWPAEFLSGFSSSSSGNVL

SEQ ID NO: 104 - DNA, 809 bp, Zea mays
cattttgtctcctcctcgttggccaccatagaggtggggagggaatggagaggaaatattcatatgctcttttgcgaaatctctcttctttgcttgaatataccatgccatctcacttttggtttcattcgctgatatcaatttcacctcgcgtgcctttcttctctttaattgttgtgtgatttggaaatgacgctcctttcttcagcaacaacaagctcaccatggccaccttaagtggcaccggaggaggtggaggcggcaacgtcgacgcgcatgcgcaccatcatcagctgccgtcgatgccacctcctgctcgtgttggggctctcatggctcctcgtcctaacatggcggctgtggtcgccgcgagcggcggcagcacgagcgacggtagtgggccaactggcggtggttccgttatccggtcgggctccatgaccgagcgggctaggctcgccaagatcccgcagccggagccggggctcaagtgcccgcgttgcgagtccaccaacaccaagttttgttactttaacaactactcgctctcccagccgcgccacttctgcaagacgtgtcgtcgctactggactcgtggcggcgcgcttcggaacatccctgtcggtggcgggtgtcgccgcaacaagcgcaccaagtcgtccaactcgtcgtcggccacctccgcttcaagagcgagcgggacatcgtcgtccacctcgtccaccgcgaccggcggcaacagcgccgccagcatcaagctgcctcaccaggggcacggagggcagctgcctttcctagcctcgtgcacc

SEQ ID NO: 105 - DNA, 585 bp, Zea mays
atggccaccttaagtggcaccggaggaggtggaggcggcaacgtcgacgcgcatgcgcaccatcatcagctgccgtcgatgccacctcctgctcgtgttggggctctcatggctcctcgtcctaacatggcggctgtggtcgccgcgagcggcggcagcacgagcgacggtagtgggccaactggcggtggttccgttatccggtcgggctccatgaccgagcgggctaggctcgccaagatcccgcagccggagccggggctcaagtgcccgcgttgcgagtccaccaacaccaagttttgttactttaacaactactcgctctcccagccgcgccacttctgcaagacgtgtcgtcgctactggactcgtggcggcgcgcttcggaacatccctgtcggtggcgggtgtcgccgcaacaagcgcaccaagtcgtccaactcgtcgtcggccacctccgcttcaagagcgagcgggacatcgtcgtccacctcgtccaccgcgaccggcggcaacagcgccgccagcatcaagctgcctcaccaggggcacggagggcagctgcctttcctagcctcgtgcacc

SEQ ID NO: 106 - protein, 195 aa, Zea mays
MATLSGTGGGGGGNVDAHAHHHQLPSMPPPARVGALMAPRPNMAAVVAASGGSTSDGSGPTGGGSVIRSGSMTERARLAKIPQPEPGLKCPRCESTNTKFCYFNNYSLSQPRHFCKTCRRYWTRGGALRNIPVGGGCRRNKRTKSSNSSSATSASRASGTSSSTSSTATGGNSAASIKLPHQGHGGQLPFLASCT

SEQ ID NO: 107 - DNA, 1005 bp, Zea mays
ggcggtaccggcacgggaggcgggcgggagccggagggcctgccgtgcccgcgctgcgagtccgccaacaccaagttctgctactacaacaactacaacctgtcccagccgcgctacttctgccgggcctgccgccgctactggacgcgcgggggagcgctccgcaacgtccccgtcggcggcggcacgcgcaaggccacgcccgccggccgccgcaagcgcgccggcccggcgcccgcaaccgcggcgccgcctgcgcctccgcctccgagcgcgctccacggctcgctgctgcgcccgtacggtgggctgtccttcgccgcgccggccctggcgtcgccgctcgccgccgtggacccggaccgccggctgctggacctcggcggcagcttcacgtcgctgatcgcgccaggggccgacgtcggcgtccacttctccgccgagttcctcgtgggcggactcgcgccggcggccctgcctcgcgcgaccgcctctgtccctgctctgccgccaccgccaccgcagcagcatcagcagccgacgacggtgtcccaggcgttgccggaaggcctgttctggagcatggggtggccggacctgtccatctaagagctccatgcctccgccggtgtcccaggcgcgctcggtgcatcgtccgtcaccggtgcccggctaatgactgctgcgtgcgggtgcatcatcgctaatctagctttttctctttctctcttttagctgtttgaattaacagtagactgtagtgctgcatccaggtgatctttttgaggggggcaagttgtttagtcatcatgaaacttttccagtcaggaaggaactgtgagagagtgatctgatctgctgaggatgtcatgaaaaactctgcatgctcggttgccttgcctgcatgaggacctgtgctgcagtgcgtgagcaattgagcttgaccaaccgataccatcagcagattgttttttggggggtcgaaaacgttgagctgattggtgc

SEQ ID NO: 108 - DNA, 609 bp, Zea mays
ggcggtaccggcacgggaggcgggcgggagccggagggcctgccgtgcccgcgctgcgagtccgccaacaccaagttctgctactacaacaactacaacctgtcccagccgcgctacttctgccgggcctgccgccgctactggacgcgcgggggagcgctccgcaacgtccccgtcggcggcggcacgcgcaaggccacgcccgccggccgccgcaagcgcgccggcccggcgcccgcaaccgcggcgccgcctgcgcctccgcctccgagcgcgctccacggctcgctgctgcgcccgtacggtgggctgtccttcgccgcgccggccctggcgtcgccgctcgccgccgtggacccggaccgccggctgctggacctcggcggcagcttcacgtcgctgatcgcgccaggggccgacgtcggcgtccacttctccgccgagttcctcgtgggcggactcgcgccggcggccctgcctcgcgcgaccgcctctgtccctgctctgccgccaccgccaccgcagcagcatcagcagccgacgacggtgtcccaggcgttgccggaaggcctgttctggagcatggggtggccggacctgtccatctaa

SEQ ID NO: 109 - protein, 202 aa, Zea mays
GGTGTGGGREPEGLPCPRCESANTKFCYYNNYNLSQPRYFCRACRRYWTRGGALRNVPVGGGTRKATPAGRRKRAGPAPATAAPPAPPPPSALHGSLLRPYGGLSFAAPALASPLAAVDPDRRLLDLGGSFTSLIAPGADVGVHFSAEFLVGGLAPAALPRATASVPALPPPPPQQHQQPTTVSQALPEGLFWSMGWPDLSI

SEQ ID NO: 110 - DNA, 498 bp, Zea mays
cttcagtaaccaccgaaccatccacgagccaccgccgctggcctcgcgcaacatgctgccgcctcacgtcgagatgccgaccaccagggcggcgggcatcaagctgttcggcaaggtcatcaccacgcattatcagcagcagcagcagccgccgcagcccgtgcgccacgcgggcgccgcgccggcgtcgtcggggcgcgggagcggcggcggccccggccccgacttgctggaggaggtggcgcgggcgcgcgcggcggcggccgaggcgcggctgccgtgcccgcgctgcttgagccgggacaccaagttctgctacttcaacaactacaacgtgaaccagccgcgccacttctgccgggcctgccaccgctactggacggccggcggcgccatccgcaacgtgccggtcggctccggccgccgcaagaaccgcccggtgctgctmccgccgccgccgccgccgccgcacgctaccaccgccactactaccggcag

SEQ ID NO: 111 - DNA, 446 bp, Zea mays
atgctgccgcctcacgtcgagatgccgaccaccagggcggcgggcatcaagctgttcggcaaggtcatcaccacgcattatcagcagcagcagcagccgccgcagcccgtgcgccacgcgggcgccgcgccggcgtcgtcggggcgcgggagcggcggcggccccggccccgacttgctggaggaggtggcgcgggcgcgcgcggcggcggccgaggcgcggctgccgtgcccgcgctgcttgagccgggacaccaagttctgctacttcaacaactacaacgtgaaccagccgcgccacttctgccgggcctgccaccgctactggacggccggcggcgccatccgcaacgtgccggtcggctccggccgccgcaagaaccgcccggtgctgctmccgccgccgccgccgccgccgcacgctaccaccgccactactaccggcag

SEQ ID NO: 112 - protein, 148 aa, Zea mays
MLPPHVEMPTTRAAGIKLFGKVITTHYQQQQQPPQPVRHAGAAPASSGRGSGGGPGPDLLEEVARARAAAAEARLPCPRCLSRDTKFCYFNNYNVNQPRHFCRACHRYWTAGGAIRNVPVGSGRRKNRPVLLPPPPPPPHATTATTTG

SEQ ID NO: 113 - DNA, 761 bp, Zea mays
tgaatgcctgataccgataggcgatagctagtacgtgatagctccttcagaaaccatcgaaccatccacgagccaccgccgctggcctcgcgcaacatgctgccgcctcacgtcgagatgccgaccaccagggcggcgggcatcaagctgttcggcaaggtcatcaccacgcattatcagcagcagcagcagccgcagcccgtgcgccacgcgggcgccgcgccggcgtcgtcggggcgcgggagcggcggcggcggcggcggcggccccgacttgctggaggaggtggctcgggcgcgcgcggcggcggccgaggcgcggctgccgtgcccgcgctgcttgagccgggacaccaagttctgctacttcaacaactacaacgtgaaccagccgcgccacttctgccgggcctgccaccgctactggacggccggcggcgccatccgcaacgtgccggtcggctccggccgccgcaagaaccgcccggtgctgctccgccgccgccgcacgctgccaccgccactactaccggcagtagtgctactgtcagcgccgacgatagtaacgaccaccgccgctcggcctcggggtctcctccagtgtttacctctgcgttcaccgcgccctactacctcggctcgcccgcccagttcgccacgtcgccgccggcttacgccgctgccgcgcccccggggacgacggggcagtgcttgtggctggtggcggcgaccacaaccacggatcgtctgctgcaacggacg

SEQ ID NO: 114 - DNA, 665 bp, Zea mays
atgctgccgcctcacgtcgagatgccgaccaccagggcggcgggcatcaagctgttcggcaaggtcatcaccacgcattatcagcagcagcagcagccgcagcccgtgcgccacgcgggcgccgcgccggcgtcgtcggggcgcgggagcggcggcggcggcggcggcggccccgacttgctggaggaggtggctcgggcgcgcgcggcggcggccgaggcgcggctgccgtgcccgcgctgcttgagccgggacaccaagttctgctacttcaacaactacaacgtgaaccagccgcgccacttctgccgggcctgccaccgctactggacggccggcggcgccatccgcaacgtgccggtcggctccggccgccgcaagaaccgcccggtgctgctccgccgccgccgcacgctgccaccgccactactaccggcagtagtgctactgtcagcgccgacgatagtaacgaccaccgccgctcggcctcggggtctcctccagtgtttacctctgcgttcaccgcgccctactacctcggctcgcccgcccagttcgccacgtcgccgccggcttacgccgctgccgcgcccccggggacgacggggcagtgcttgtggctggtggcggcgaccacaaccacggatcgtctgctgcaacggacg

SEQ ID NO: 115 - protein, 221 aa, Zea mays
MLPPHVEMPTTRAAGIKLFGKVITTHYQQQQQPQPVRHAGAAPASSGRGSGGGGGGGPDLLEEVARARAAAAEARLPCPRCLSRDTKFCYFNNYNVNQPRHFCRACHRYWTAGGAIRNVPVGSGRRKNRPVLLRRRRTLPPPLLPAVVLLSAPTIVTTTAARPRGLLQCLPLRSPRPTTSARPPSSPRRRRLTPLPRPRGRRGSACGWWRRPQPRIVCCNG

SEQ ID NO: 116 - DNA, 607 bp, Zea mays
tcaaagtttaaccaaccacccttttcgccacttcacaaactttatcagcttttcaattccttgaagaagaagaagaagaaagcctcaaaattcaacccatattctccatagaaacgaaactttatcttcaaattcaatcaagcagcgagggcttgaggtgtggaggagtatttaaaatgcaagacccaatgggctttcaccaaatgaaagcgccggcttttcaagagcaagagcagcagcagctgaaatgcccccgctgtgactcaaccaacaccaaattctgttactacaacaactataacttgtctcagccccgccatttctgcaagaactgccgccgttactggactaaaggcggcgccctccgtaacatacccgtcggtggcggcacccgtaagggcaccaaacgctcctcctcctcctccaccaacaaccctaagcgccagcccaacccctctccagaccccaccccaaaccaaaaaatccctgatccctctccgccgccgccgacatcatcatcatcgtcgatgtttccccagcagattgttttgagctcgggggctcagaattcggacttggatatcgactcgacccggatgtatctgtt

SEQ ID NO: 117 - DNA, 431 bp, Zea mays
atgcaagacccaatgggctttcaccaaatgaaagcgccggcttttcaagagcaagagcagcagcagctgaaatgcccccgctgtgactcaaccaacaccaaattctgttactacaacaactataacttgtctcagccccgccatttctgcaagaactgccgccgttactggactaaaggcggcgccctccgtaacatacccgtcggtggcggcacccgtaagggcaccaaacgctcctcctcctcctccaccaacaaccctaagcgccagcccaacccctctccagaccccaccccaaaccaaaaaatccctgatccctctccgccgccgccgacatcatcatcatcgtcgatgtttccccagcagattgttttgagctcgggggctcagaattcggacttggatatcgactcgacccggatgtatctgtt

SEQ ID NO: 118 - protein, 143 aa, Zea mays
MQDPMGFHQMKAPAFQEQEQQQLKCPRCDSTNTKFCYYNNYNLSQPRHFCKNCRRYWTKGGALRNIPVGGGTRKGTKRSSSSSTNNPKRQPNPSPDPTPNQKIPDPSPPPPTSSSSSMFPQQIVLSSGAQNSDLDIDSTRMYL

SEQ ID NO: 119 - DNA, 1583 bp, Zea mays
agtttcgtcatgcacaagcatgacgaactggtttctctctccgtttcctaattattattttagagtgaatccggcctttttccaatacgaacttcaaatctctggttgattgatcgctgtgattcgcaagtttgtttactacctatgctcatgccctggttgagcatagacacaaaggaagggcatcattataggcatgttcttgaatataccgtgctttgaattccagcctttattaattgattcgttatacatacagatgcagatgcagatgcagcagcagtcaccgctccagtgtctcctcggcagcggcggtggcagcgaccaccaccacctcatgcctcctccgtccggcctggcgccgctgccggggggtcctgctgacacagcggcgagcggtccggcgggaggcggctcgtccacctcggcctcagtgcaagccgcggcgggagcgggggcaggggcgcagcctcgccccgtggtgtcgatggcggagcgcgcccggctcgcgcgcgtgccgctgccggagcccggcacgctccgatgcccgcgctgtgactcgaccaacaccaagttctgctacttcaataactactcgctgtcgcagccgcgccacttctgcaaggcgtgccgccgctactggacccgtggcggcgcgctccgcaacgtgcccgtcggcggcgggtgcaggcgcaacaccaagcgctccagcaagaagtcgtcccgcggcggcggcgcgggcgccacggcggcaacctcctcgtcctcgaccacctccacctccaccacggccactaccaccaccaccacgagcgcggccatggcggcggccgaggccatcgccggcatgcaggcgcagctgccccacctcggcctcccgcccgcagcggccgccgcggcgcttgaggcctcgctggagggctaccaccactacctcccgctccagatgcagccgcagttcctgcagcaggctggcctgcacggctaccatttcgccgacgacggcaccggcgtcctcgcagctgacgggttcccgaggggcgtcgtcgcctcggggctgctcgcgcagctcgcggcggtgaagatggaggagcacggcagcaacggcggaggtgccatcgcggcgcatcatgagcagcagtcctactggcccggcagcaccggcggtggcggtgggtggccggtggagttcttgtcggggttcagctcatcctcgtcggggaatgtgttgtgagttgcatgtggccgcgtccaggtccacctgggcatgcatgatggggggcatgcatgcatggatgctgatgaagctgaactagctcaccatcctcaattagttctagggtttatgtggtgtttggaacttaattggcgtcgtcttcttcaattaggctggtgatgatcttgtgttggggtaaggtttttacatgttttttttcttatgagttttaagctggttaatgtcgtgcagttggtagttttgcatgtagtgggaagtgcatgtgttgactcatggaggttatatgtgtcaggttacttaactatactatgtttaattatatgagaacttggtgggcatagttgta

SEQ ID NO: 120 - DNA, 1038 bp, Zea mays
atgttcttgaatataccgtgctttgaattccagcctttattaattgattcgttatacatacagatgcagatgcagatgcagcagcagtcaccgctccagtgtctcctcggcagcggcggtggcagcgaccaccaccacctcatgcctcctccgtccggcctggcgccgctgccggggggtcctgctgacacagcggcgagcggtccggcgggaggcggctcgtccacctcggcctcagtgcaagccgcggcgggagcgggggcaggggcgcagcctcgccccgtggtgtcgatggcggagcgcgcccggctcgcgcgcgtgccgctgccggagcccggcacgctccgatgcccgcgctgtgactcgaccaacaccaagttctgctacttcaataactactcgctgtcgcagccgcgccacttctgcaaggcgtgccgccgctactggacccgtggcggcgcgctccgcaacgtgcccgtcggcggcgggtgcaggcgcaacaccaagcgctccagcaagaagtcgtcccgcggcggcggcgcgggcgccacggcggcaacctcctcgtcctcgaccacctccacctccaccacggccactaccaccaccaccacgagcgcggccatggcggcggccgaggccatcgccggcatgcaggcgcagctgccccacctcggcctcccgcccgcagcggccgccgcggcgcttgaggcctcgctggagggctaccaccactacctcccgctccagatgcagccgcagttcctgcagcaggctggcctgcacggctaccatttcgccgacgacggcaccggcgtcctcgcagctgacgggttcccgaggggcgtcgtcgcctcggggctgctcgcgcagctcgcggcggtgaagatggaggagcacggcagcaacggcggaggtgccatcgcggcgcatcatgagcagcagtcctactggcccggcagcaccggcggtggcggtgggtggccggtggagttcttgtcggggttcagctcatcctcgtcggggaatgtgttgtga

SEQ ID NO: 121 - protein, 345 aa, Zea mays
MFLNIPCFEFQPLLIDSLYIQMQMQMQQQSPLQCLLGSGGGSDHHHLMPPPSGLAPLPGGPADTAASGPAGGGSSTSASVQAAAGAGAGAQPRPVVSMAERARLARVPLPEPGTLRCPRCDSTNTKFCYFNNYSLSQPRHFCKACRRYWTRGGALRNVPVGGGCRRNTKRSSKKSSRGGGAGATAATSSSSTTSTSTTATTTTTTSAAMAAAEAIAGMQAQLPHLGLPPAAAAAALEASLEGYHHYLPLQMQPQFLQQAGLHGYHFADDGTGVLAADGFPRGVVASGLLAQLAAVKMEEHGSNGGGAIAAHHEQQSYWPGSTGGGGGWPVEFLSGFSSSSSGNVL

SEQ ID NO: 122 - DNA, 1491 bp, Zea mays
agcgggggggcgcatcacaggtccggcaccgacatgcgcagagcgtcaccgtggcactcccactcccaccgcagcaagcgcgcgccgttcgacgccgccctctccgccgccaacaacaagctgccctggccgtcgccgcggcgcccggcgaaccatgccgtcctcctttctgacctcttcttcctcttccgcttcgtcgcacctctcttacctgatccctgctagggcggcgccgccgcctccctttgccatggtacgtacgtaccttaaagagtaccacaaacgtcctgatccgtcgatcgtgtagcgtactctctgatacatctcttccacagttccactcagacatgcatgcatgcacatgcgatgactagctagcggtgtgttcctgttcctggcagggacagggatacggtgccaccagtggcggcggcgtggcgaacgccacgagtgccgcggcggcgcctccgcctccgaggcagggagccagcaggaacgcgggcgcggggcacccgccgctgccgcgcccgccgccgcggcagtgcccgcgctgccggtccgccaacaccaagttctgctactacaacaactacagccgcgagcagccacggtacctctgcaaggcgtgccgccgccactggaccgagggcggcacgctccgcgacgtgcccgtcggcggcggccgcaagaacaggcgcggcgccaagggcggcgccgctgcgaaagggtctgcctcggccgcggccgcggctccggcgcagcagggagggaacgtcctcggcgccgacacgttcccaggggacttgctgcggcagctggtgcagttccagccggacgcggccgtgggcgggggcggctacgccatcgacctgagcgcgtggcaccaaatggtcgctgccacggcgccgccgccgccgccgccgggaactggcggcgatgtcagcagtctcggtctcggagcggcgggggcgggagcgggagcggaggccaactgcgtcgcgttgcagtactggagcgaagacggcatgcccggtcttgatggggcctgctaagtgcgtactactactagagctttgttattactattacggtattaccacaggagtatgttccagcctacttatatatgtgtgcgtgtactgcgtgcgaaacatacatactagttagtatatatgatcagcttgttttctctacttccttgtggcgcgtactatgtattcttagctaaataaaattgtgcgcgtgctaataaactacgctgctccaaccatcctaatgcctaaggtgtggttcacccttgtccgtttagagttctggacgactggaccttgcaaaaattgtgaccatatttttatttcatgctagcttgctacccttctgtcctaaaattaaacgacttcttgagtcgtcggaagtcaaagtatttaaatcttaactaaatttatagaaaacagcattaatatttatgacacaaaattaatatcgatatca

SEQ ID NO: 123 - DNA, 678 bp, Zea mays
ctagcggtgtgttcctgttcctggcagggacagggatacggtgccaccagtggcggcggcgtggcgaacgccacgagtgccgcggcggcgcctccgcctccgaggcagggagccagcaggaacgcgggcgcggggcacccgccgctgccgcgcccgccgccgcggcagtgcccgcgctgccggtccgccaacaccaagttctgctactacaacaactacagccgcgagcagccacggtacctctgcaaggcgtgccgccgccactggaccgagggcggcacgctccgcgacgtgcccgtcggcggcggccgcaagaacaggcgcggcgccaagggcggcgccgctgcgaaagggtctgcctcggccgcggccgcggctccggcgcagcagggagggaacgtcctcggcgccgacacgttcccaggggacttgctgcggcagctggtgcagttccagccggacgcggccgtgggcgggggcggctacgccatcgacctgagcgcgtggcaccaaatggtcgctgccacggcgccgccgccgccgccgccgggaactggcggcgatgtcagcagtctcggtctcggagcggcgggggcgggagcgggagcggaggccaactgcgtcgcgttgcagtactggagcgaagacggcatgcccggtcttgatggggcctgctaa

SEQ ID NO: 124 - protein, 225 aa, Zea mays
LAVCSCSWQGQGYGATSGGGVANATSAAAAPPPPRQGASRNAGAGHPPLPRPPPRQCPRCRSANTKFCYYNNYSREQPRYLCKACRRHWTEGGTLRDVPVGGGRKNRRGAKGGAAAKGSASAAAAAPAQQGGNVLGADTFPGDLLRQLVQFQPDAAVGGGGYAIDLSAWHQMVAATAPPPPPPGTGGDVSSLGLGAAGAGAGAEANCVALQYWSEDGMPGLDGAC

SEQ ID NO: 125 - DNA, 947 bp, Zea mays
cagcgagtgctagctttgttctgattcttagtaaccaagccatgcatatgcctcctcttaccgttctgctaagctaatacatattgaatcgtctgcaaagtaactgcatgcaagagaaatgtttgaaggacctcgtcggtaaatttatctgctcatgtaaattaaatgaacatgttacatttcttttggaatgtttctttagctagtttgtaaaaaaattcactgaaatcttgcagacaaccaaccaaggcatggcgtatgtgttgtgctgaaacgtcgaccggtaccgcggcccaaaaggcaccagcaccacaggcatgctagctatatatatactacaagggcaagtctactggatgcctaatagtacgtgacagctccttcagaaacatcgaaaaccaccgaaccagtcaccaccacgcctcggcctcgcgcaacatgctgcctcatgtcgagatgccggtaccgggcagggcggcggccgcgtgcaccggcgcggcgggcatcaagctgttcggcaaggtcatcaccacgccgccgcaccacgcgggcgccgcgcctccgaggctgcagcaggcggcagcggcgtcggggcgcgggagcgccgacctactggaggaggtggcgcgggcgcgcgcggcggcggccgaggtgcggctgccgtgcccgcgctgcctgagccgggacaccaagttctgctacttcaacaactacaacgtcaaccagccgcgccacttctgccgggcctgccaccgctactggacggccggcggcgccatccgcaacgtgcccatcggctccggccgccgcarraaccgcccggtgctgctcccaccgcggcgccgcacgctgtcaccgccaccgataacagcsgcggtcgggcgcggggtctcctccggtgttcggctctgtgttcaccgcgccctaccagcagctcggcgcgccca

SEQ ID NO: 126 - DNA, 509 bp, Zea mays
atgctgcctcatgtcgagatgccggtaccgggcagggcggcggccgcgtgcaccggcgcggcgggcatcaagctgttcggcaaggtcatcaccacgccgccgcaccacgcgggcgccgcgcctccgaggctgcagcaggcggcagcggcgtcggggcgcgggagcgccgacctactggaggaggtggcgcgggcgcgcgcggcggcggccgaggtgcggctgccgtgcccgcgctgcctgagccgggacaccaagttctgctacttcaacaactacaacgtcaaccagccgcgccacttctgccgggcctgccaccgctactggacggccggcggcgccatccgcaacgtgcccatcggctccggccgccgcarraaccgcccggtgctgctcccaccgcggcgccgcacgctgtcaccgccaccgataacagcsgcggtcgggcgcggggtctcctccggtgttcggctctgtgttcaccgcgccctaccagcagctcggcgcgccca

SEQ ID NO: 127 - protein, 169 aa, Zea mays
  VARIANT 125: Xaa = Any Amino Acid
MLPHVEMPVPGRAAAACTGAAGIKLFGKVITTPPHHAGAAPPRLQQAAAASGRGSADLLEEVARARAAAAEVRLPCPRCLSRDTKFCYFNNYNVNQPRHFCRACHRYWTAGGAIRNVPIGSGRRXNRPVLLPPRRRTLSPPPITAAVGRGVSSGVRLCVHRALPAARRA

SEQ ID NO: 128 - DNA, 1065 bp, Zea mays
cttcctttctactactggtgtttcctcgctttgacggagttggttggttccagtctccaaatggtgtcccacgtcgagatgggcctcgctgccggcgggttcaagctcttcggcaaggtcatcacgcagtgcgccgagagcgctccgccggcctccttggtcgcgcgggagagggacgatccggacgagcgcgaccagccggtggtgaagcgggaggcagcggcggcgtcggacagcgactcggccgtcgtactcgtagacaagcagcggcgctccggcgggccggccgagagcgaggacagcaaggggcagcatcacccgcgcccgcggcagccgcagcagcaggacaccgcggaggcccgcgccgcggcgtcggcgccgccgctgccgtgcccgcggtgccggagccgcaacaccaagttctgctacttcaacaactacaacgtcaaccagccgcgccacttctgcaaggactgccaccgctactggaccgcgggcggcgcgctccgcaacgtccccgtcggcgccggccgccgcaagaaccggcccctcggcgccgcacccatccccttggccgtgtccgtgccggcccagcacctgcagcacccgcagcaccccgctgcggcggcgggccacggcctcggcctccccggccagcacccttcctgcccgacgtcgccgtccccggcctacgccgggcggtggccggtctgcccggaccgccggttctgagctgacgtgacgatccgatcgatggatcgatcgatcgcagacctctcgggagtcggatgtagcgccaggttggttttgacctgcgtaattagcgggcggggtgggggacggaacggaaggctgccttgtttgtactgtacgtgcttgaggcccaaaagggggtatggattattgatggaggacgacatggaaatgtcttatgttttcttctctgtctagcgtgtactagtagctcgtctgtttccttcttttgttgttccctaaccctgcactactagtactagttataagctaccagatgtgtgtaaaaagtgtggtactactaataatgta

SEQ ID NO: 129 - DNA, 732 bp, Zea mays
cttcctttctactactggtgtttcctcgctttgacggagttggttggttccagtctccaaatggtgtcccacgtcgagatgggcctcgctgccggcgggttcaagctcttcggcaaggtcatcacgcagtgcgccgagagcgctccgccggcctccttggtcgcgcgggagagggacgatccggacgagcgcgaccagccggtggtgaagcgggaggcagcggcggcgtcggacagcgactcggccgtcgtactcgtagacaagcagcggcgctccggcgggccggccgagagcgaggacagcaaggggcagcatcacccgcgcccgcggcagccgcagcagcaggacaccgcggaggcccgcgccgcggcgtcggcgccgccgctgccgtgcccgcggtgccggagccgcaacaccaagttctgctacttcaacaactacaacgtcaaccagccgcgccacttctgcaaggactgccaccgctactggaccgcgggcggcgcgctccgcaacgtccccgtcggcgccggccgccgcaagaaccggcccctcggcgccgcacccatccccttggccgtgtccgtgccggcccagcacctgcagcacccgcagcaccccgctgcggcggcgggccacggcctcggcctccccggccagcacccttcctgcccgacgtcgccgtccccggcctacgccgggcggtggccggtctgcccggaccgccggttctga

SEQ ID NO: 130 - protein, 243 aa, Zea mays
LPFYYWCFLALTELVGSSLQMVSHVEMGLAAGGFKLFGKVITQCAESAPPASLVARERDDPDERDQPVVKREAAAASDSDSAVVLVDKQRRSGGPAESEDSKGQHHPRPRQPQQQDTAEARAAASAPPLPCPRCRSRNTKFCYFNNYNVNQPRHFCKDCHRYWTAGGALRNVPVGAGRRKNRPLGAAPIPLAVSVPAQHLQHPQHPAAAAGHGLGLPGQHPSCPTSPSPAYAGRWPVCPDRRF

SEQ ID NO: 131 - DNA, 786 bp, Zea mays
gaatgtgatgccccgcacacatgagatgccattcaataataggaacccccgggcgtcttccctcctcgccaggaaaccctcctcaccgttgcaatatttgcttggcatacttccaatccttccttatatacgtgaacgcacccacctaatcacctgatacctagctgcctgctcgtggcaccagcaccaccgatcgccaagctagctaggacggccgaccgatcgatcgagctgctgcaagggcggccgccggcgactctcgatcgctaggcgctagctgcccgccggcgccggtacgtatatatggcgacgggggacgacgcggtcgggacacggaagggcggcgccggcaccggcggtgcagctggcggcgggggcacgcctaccacgcagacgcaacagcagcagcagcagacgcccccgccgcccgagcagggggtgagctgcccgcgctgcgactccccgaacaccaagttctgctactacaacaactacagcctgtcgcagccgcgccacttctgcaagacgtgccgcaggtactggaccaagggcggcgcgctccgcaacgtgcccgtaggcgggggctgccgcaagaacaagcgctcgcgctccgcggcggccgcgcgcctctcgctcaacctgccgctggaggccgccgccgaccagcaggcggcgaggctgggcttccttggcgccgccgctcatccggtgttgtcgtcgtcgtcgatcggcggcggcggccccgcggccgactaccaccaaccgcaggtgggcgccgccgtcggg

SEQ ID NO: 132 - DNA, 483 bp, Zea mays
atggcgacgggggacgacgcggtcgggacacggaagggcggcgccggcaccggcggtgcagctggcggcgggggcacgcctaccacgcagacgcaacagcagcagcagcagacgcccccgccgcccgagcagggggtgagctgcccgcgctgcgactccccgaacaccaagttctgctactacaacaactacagcctgtcgcagccgcgccacttctgcaagacgtgccgcaggtactggaccaagggcggcgcgctccgcaacgtgcccgtaggcgggggctgccgcaagaacaagcgctcgcgctccgcggcggccgcgcgcctctcgctcaacctgccgctggaggccgccgccgaccagcaggcggcgaggctgggcttccttggcgccgccgctcatccggtgttgtcgtcgtcgtcgatcggcggcggcggccccgcggccgactaccaccaaccgcaggtgggcgccgccgtcggg

SEQ ID NO: 133 - protein, 161 aa, Zea mays
MATGDDAVGTRKGGAGTGGAAGGGGTPTTQTQQQQQQTPPPPEQGVSCPRCDSPNTKFCYYNNYSLSQPRHFCKTCRRYWTKGGALRNVPVGGGCRKNKRSRSAAAARLSLNLPLEAAADQQAARLGFLGAAAHPVLSSSSIGGGGPAADYHQPQVGAAVG

SEQ ID NO: 134 - DNA, 534 bp, Zea mays
cagcaacagcacgctcaacatggccagcagcttccaagtggcggcaacggcggcggcggcggcggcatggacacgcatgcgcatcatcatcatcatcagctgccgccagtgccacctcctcctagcggggctctcatggctcctcgccctgacatgtcggctatggtcgtccctgcgagcggtggtggtggcgggccgactagcggcggcaccgctatccgtccgggctccatgaccgagcgggccaagctcgccaagatcccgcagccggagccggggctcaagtgcccgcgctgcgagtccaccaacaccaagttctgctacttcaacaactactcgctctcccagccgcgccacttctgcaagacgtgccgccggtactggacgcgcggcggcgcgctccggaacgtccccgtgggcggcgggtgccgccgtaacaagcgcaccaagtcgtccaagtccaactcgtcatcggccgcctctgcttcaggcggcgcgggcgggacgtcgtcgtccacctcgtccaccggacgg

SEQ ID NO: 135 - protein, 178 aa, Zea mays
QQQHAQHGQQLPSGGNGGGGGGMDTHAHHHHHQLPPVPPPPSGALMAPRPDMSAMVVPASGGGGGPTSGGTAIRPGSMTERAKLAKIPQPEPGLKCPRCESTNTKFCYFNNYSLSQPRHFCKTCRRYWTRGGALRNVPVGGGCRRNKRTKSSKSNSSSAASASGGAGGTSSSTSSTGR

SEQ ID NO: 136 - DNA, 1574 bp, Zea mays
ccatagaggtggggagggaatggagaggaaatatccctaggctcttttgtgaaatctctcctctttgcttgaatataccaagtcgtctctcttttggtttcattcgctgagatatcaatttcaccttgcgcgcctttcttccctttaattgttgtgtgattgtgtgtttgatttgtaaatgacgttgctttcttcagcaactgcaagctcaccatggccagcttccaagtgacgccggaggaggtggaggcggtggcgtcgacgtgcatgcgcaccatcatcagctaccgccgatgccaccttctgctcctggtggggctctcatggctcctcgccgtgacatggcggctgtggtcgccgcgagcggtggcagcacgagcggcggcggtgggccgactggcggtggttcccctatccgtccgggctccatgaccgagcgggctaggctcgccaagatcccgcagccagagccggggctcaagtgcccgcgttgcgagtccaccaacaccaagttttgctacttcaacaactactcgctctcccagccgcgccacttctgcaagacgtgtcgccggtactggacgcgcggcggcacgctccggaacgtccccgttggcggcgggtgccgccgcaacaagcgcaccaagtcgtccaagtccaactcgtcgtcagccgcctccgcttccagcgcgggtgggacgtcgtcgtccacctcgtccaccgcgaccggtggcagcggcagcgccgccagcatcatgccgcctcaccaggggcacgggggacagccgcagttcctagcctcgttgcaccaccctctcgtcggcggggaccactacagcaccggcgcgtcaagattagggttccccgggctgagctcgctggaccccatggactaccaccagttcggcgccggtgccggaggtgccatcgggctggagcagtggcgcctaccgcagatacaacagttcccattcctaagcagccgccccgacgccgtgcaaccgccaatgtctggcatttacccgttcgaagtggaaggccacggcggggaaggttccggcttcgctggtcacatgcttggtgaccccaaggtgtccggctcagccggactgatcacgcagctggcgtcagtgaagatggaggacaacccaacgtccgcagcgatggcaagcagctcaccgagggagttccttggtctccctaggaacctccaattatggggcgctagcggcaacggcggtgcaagtggcaacaatggaggaggcaccggcaacgctgccggtggcggcgacactattcctccgggtagcagctgggtagacctgtcaggattcaactcgtcttcgtccgggaacgtgctgtgacgtgacgcgccccctgaatctgatctgcatgataagctggtacagttgctgcagatgaatggtagctctatctatttatgcaagcactgaagtttcattttagtcattgcttatgagaaagttccacagaaaaggagttgtggtgcaagattatagagggggggckgsrkggrkgkatmtatrygmtagctgatacaacgacctgttgca

SEQ ID NO: 137 - DNA, 1071 bp, Zea mays
atgccaccttctgctcctggtggggctctcatggctcctcgccgtgacatggcggctgtggtcgccgcgagcggtggcagcacgagcggcggcggtgggccgactggcggtggttcccctatccgtccgggctccatgaccgagcgggctaggctcgccaagatcccgcagccagagccggggctcaagtgcccgcgttgcgagtccaccaacaccaagttttgctacttcaacaactactcgctctcccagccgcgccacttctgcaagacgtgtcgccggtactggacgcgcggcggcacgctccggaacgtccccgttggcggcgggtgccgccgcaacaagcgcaccaagtcgtccaagtccaactcgtcgtcagccgcctccgcttccagcgcgggtgggacgtcgtcgtccacctcgtccaccgcgaccggtggcagcggcagcgccgccagcatcatgccgcctcaccaggggcacgggggacagccgcagttcctagcctcgttgcaccaccctctcgtcggcggggaccactacagcaccggcgcgtcaagattagggttccccgggctgagctcgctggaccccatggactaccaccagttcggcgccggtgccggaggtgccatcgggctggagcagtggcgcctaccgcagatacaacagttcccattcctaagcagccgccccgacgccgtgcaaccgccaatgtctggcatttacccgttcgaagtggaaggccacggcggggaaggttccggcttcgctggtcacatgcttggtgaccccaaggtgtccggctcagccggactgatcacgcagctggcgtcagtgaagatggaggacaacccaacgtccgcagcgatggcaagcagctcaccgagggagttccttggtctccctaggaacctccaattatggggcgctagcggcaacggcggtgcaagtggcaacaatggaggaggcaccggcaacgctgccggtggcggcgacactattcctccgggtagcagctgggtagacctgtcaggattcaactcgtcttcgtccgggaacgtgctgtga

SEQ ID NO: 138 - protein, 356 aa, Zea mays
MPPSAPGGALMAPRRDMAAVVAASGGSTSGGGGPTGGGSPIRPGSMTERARLAKIPQPEPGLKCPRCESTNTKFCYFNNYSLSQPRHFCKTCRRYWTRGGTLRNVPVGGGCRRNKRTKSSKSNSSSAASASSAGGTSSSTSSTATGGSGSAASIMPPHQGHGGQPQFLASLHHPLVGGDHYSTGASRLGFPGLSSLDPMDYHQFGAGAGGAIGLEQWRLPQIQQFPFLSSRPDAVQPPMSGIYPFEVEGHGGEGSGFAGHMLGDPKVSGSAGLITQLASVKMEDNPTSAAMASSSPREFLGLPRNLQLWGASGNGGASGNNGGGTGNAAGGGDTIPPGSSWVDLSGFNSSSSGNVL

SEQ ID NO: 139 - DNA, 1382 bp, Zea mays
  misc_feature 182, 183, 184: n = A,T,C or G
cagcaacagcacgctcaacatggccagcagcttccaagtggcggcaacggcggcggcggcggcggcatggacacgcatgcgcatcatcatcatcatcagctgccgccggtgccacctcctcctagcggggctctcatggctcctcgccctgacatgtcggctatggtccctgctgcgagcgnnngtggtggcgggccgactagcggcggcaccgctatccgtccgggctccatgaccgagcgggccaagctcgccaagatcccgcagccggagccggggctcaagtgcccgcgctgcgagtccaccaacaccaagttctgctacttcaacaactactcgctctcccagccgcgccacttctgcaagacgtgccgccggtactggacgcgcggcggcgcgctccggaacgtccccgtgggcggcgggtgccgccgtaacaagcgcaccaagtcgtccaagtccaactcgtcatcggccgccgcctctgcttcaggcggcgcgggcgggacgtcgtcgtccacctcgtccaccgctaccggcggcagcagcagcgcgggcgccatcatgccgtctcatcaggggcagctgcagccgttcctggcctcgttgcaccaccctcttgccggcggcggcggcggcggggatcactacagcaccggcgcgtcaaggttagggttcccaggactgagctcgctggaccccatggactaccaccagttcggcgcgggcgcgggcgccggcgccagcagcgctgccatcgggctagagcagtggcgtctgccgcatatacagcagttccccttcctaagcggccgccccgaccccgtgcaaccaacaatgtctagcatttacccgttcgacttggaagggcacggcggggacgctcccggcttccccggcgggcacatgctaggtgcctccaaggtgcccggctcagccggactaatcacgcagctggcatcggttaagatggaggacaacccagcgtctgcggcgatggcgagcagctcgccgagggagttccttagcctccctgggaacctccaattctggggcggcggcggcaacaacaacggcggtgcaagtggcaacaatggaggaggcgctggcaacggcggtggtggtggcggcggtggcgctgtcgctccgggcagcagctgggtggacctgtcaggattcaactcgtcttcgtctgggaacgtcctgtgacatgccctctggtctgcatgctagctgcattaaaaaggtagccctattatttatgcaaagccttgaatcttcattttagtcatggcttctgagaaagttcaacagaaaaaaggagttgtggttgcaagattataggggctatatatatctgatagctatcgtctgcggtgacg

SEQ ID NO: 140 - DNA, 1206 bp, Zea mays
cagcaacagcacgctcaacatggccagcagcttccaagtggcggcaacggcggcggcggcggcggcatggacacgcatgcgcatcatcatcatcatcagctgccgccggtgccacctcctcctagcggggctctcatggctcctcgccctgacatgtcggctatggtccctgctgcgagcggtggtggcgggccgactagcggcggcaccgctatccgtccgggctccatgaccgagcgggccaagctcgccaagatcccgcagccggagccggggctcaagtgcccgcgctgcgagtccaccaacaccaagttctgctacttcaacaactactcgctctcccagccgcgccacttctgcaagacgtgccgccggtactggacgcgcggcggcgcgctccggaacgtccccgtgggcggcgggtgccgccgtaacaagcgcaccaagtcgtccaagtccaactcgtcatcggccgccgcctctgcttcaggcggcgcgggcgggacgtcgtcgtccacctcgtccaccgctaccggcggcagcagcagcgcgggcgccatcatgccgtctcatcaggggcagctgcagccgttcctggcctcgttgcaccaccctcttgccggcggcggcggcggcggggatcactacagcaccggcgcgtcaaggttagggttcccaggactgagctcgctggaccccatggactaccaccagttcggcgcgggcgcgggcgccggcgccagcagcgctgccatcgggctagagcagtggcgtctgccgcatatacagcagttccccttcctaagcggccgccccgaccccgtgcaaccaacaatgtctagcatttacccgttcgacttggaagggcacggcggggacgctcccggcttccccggcgggcacatgctaggtgcctccaaggtgcccggctcagccggactaatcacgcagctggcatcggttaagatggaggacaacccagcgtctgcggcgatggcgagcagctcgccgagggagttccttagcctccctgggaacctccaattctggggcggcggcggcaacaacaacggcggtgcaagtggcaacaatggaggaggcgctggcaacggcggtggtggtggcggcggtggcgctgtcgctccgggcagcagctgggtggacctgtcaggattcaactcgtcttcgtctgggaacgtcctgtga

SEQ ID NO: 141 - protein, 402 aa, Zea mays
  VARIANT 61, 62: Xaa = Any Amino Acid
QQQHAQHGQQLPSGGNGGGGGGMDTHAHHHHHQLPPVPPPPSGALMAPRPDMSAMVPAASXXGGGPTSGGTAIRPGSMTERAKLAKIPQPEPGLKCPRCESTNTKFCYFNNYSLSQPRHFCKTCRRYWTRGGALRNVPVGGGCRRNKRTKSSKSNSSSAAASASGGAGGTSSSTSSTATGGSSSAGAIMPSHQGQLQPFLASLHHPLAGGGGGGDHYSTGASRLGFPGLSSLDPMDYHQFGAGAGAGASSAAIGLEQWRLPHIQQFPFLSGRPDPVQPTMSSIYPFDLEGHGGDAPGFPGGHMLGASKVPGSAGLITQLASVKMEDNPASAAMASSSPREFLSLPGNLQFWGGGGNNNGGASGNNGGGAGNGGGGGGGGAVAPGSSWVDLSGFNSSSSGNVL

SEQ ID NO: 142 - DNA, 1075 bp, Zea mays
tgccaccttctgctcctggtggggctctcatggctcctcgccgtgacatggcggctgtggtcgccgcgagcggtggcagcacgagcggcggcggtgggccgactggcggtggtttccctatccgtccgggctccatgaccgagcgggctaggctcgccaagatcccgcagccagagccggggctcaagtgcccgcgttgcgagtccaccaacaccaagttttgctacttcaacaactactcgctctcccagccgcgccacttctgcaagacgtgtcgccggtactggacgcgcggcggcacgctccggaacgtccccgttggcggcgggtgccgccgcaacaagcgcaccaagtcgtccaagtccaactcgtcgtcagccgcctccgcttccagcgcgggtgggacgtcgtcgtccacctcgtccaccgcgaccggtggcagcggcagcgccgccagcatcatgccgcctcaccaggggcacgggggacagccgcagttcctagcctcgttgcaccaccctctcgtcggcggggaccactacagcaccggcgcgtcaagattagggttccccgggctgagctcgctggaccccatggactaccaccagttcggcgccggtgccggaggtgccatcgggctggagcagtggcgcctaccgcagatacaacagttcccattcctaagcagccgccccgacgccgtgcaaccgccaatgtctggcatttacccgttcgaagtggaaggccacggcggggaaggttccggcttcgctggtcacatgcttggtgaccccaaggtgtccggctcagccggactgatcacgcagctggcgtcagtgaagatggaggacaacccaacgtccgcagcgatggcaagcagctcaccgagggagttccttgggtctccctaggaacctcccaattatggggcgctagcggcaacggcggtgcaagtggcaacaatggaggaggcaccggcaacgctgccggtggcggcgacactattcctccgggkrstcaagctggrtwgayctgwcagsakwsmacwmgkcttcgtccgggaacgtgctggacg

SEQ ID NO: 143 - DNA, 909 bp, Zea mays
ccaccttctgctcctggtggggctctcatggctcctcgccgtgacatggcggctgtggtcgccgcgagcggtggcagcacgagcggcggcggtgggccgactggcggtggtttccctatccgtccgggctccatgaccgagcgggctaggctcgccaagatcccgcagccagagccggggctcaagtgcccgcgttgcgagtccaccaacaccaagttttgctacttcaacaactactcgctctcccagccgcgccacttctgcaagacgtgtcgccggtactggacgcgcggcggcacgctccggaacgtccccgttggcggcgggtgccgccgcaacaagcgcaccaagtcgtccaagtccaactcgtcgtcagccgcctccgcttccagcgcgggtgggacgtcgtcgtccacctcgtccaccgcgaccggtggcagcggcagcgccgccagcatcatgccgcctcaccaggggcacgggggacagccgcagttcctagcctcgttgcaccaccctctcgtcggcggggaccactacagcaccggcgcgtcaagattagggttccccgggctgagctcgctggaccccatggactaccaccagttcggcgccggtgccggaggtgccatcgggctggagcagtggcgcctaccgcagatacaacagttcccattcctaagcagccgccccgacgccgtgcaaccgccaatgtctggcatttacccgttcgaagtggaaggccacggcggggaaggttccggcttcgctggtcacatgcttggtgaccccaaggtgtccggctcagccggactgatcacgcagctggcgtcagtgaagatggaggacaacccaacgtccgcagcgatggcaagcagctcaccgagggagttccttgggtctccctag

SEQ ID NO: 144 - protein, 302 aa, Zea mays
PPSAPGGALMAPRRDMAAVVAASGGSTSGGGGPTGGGFPIRPGSMTERARLAKIPQPEPGLKCPRCESTNTKFCYFNNYSLSQPRHFCKTCRRYWTRGGTLRNVPVGGGCRRNKRTKSSKSNSSSAASASSAGGTSSSTSSTATGGSGSAASIMPPHQGHGGQPQFLASLHHPLVGGDHYSTGASRLGFPGLSSLDPMDYHQFGAGAGGAIGLEQWRLPQIQQFPFLSSRPDAVQPPMSGIYPFEVEGHGGEGSGFAGHMLGDPKVSGSAGLITQLASVKMEDNPTSAAMASSSPREFLGSP

SEQ ID NO: 145 - protein, 40 aa, Artificial Sequence
  DOF domain consensus sequence
CPRCXSXDTKFCYFNNYNXXQPRHFCKXCRRYWTXGGTLR

SEQ ID NO: 146 - DNA, 792 bp, Zea mays
  misc_feature 377: n = A,T,C or G
tcttyctcctcctcctcctcctccgcccccgccgcagcctctgcccctgcagcaggcggccggggccgggggcaccsgcggcgagccgctgccgtgcccscggtgcggcagccgggagaccaagttctgctacttcaacaactacaacgtgcggcagccgcgccacctctgccgcgcctgccgccgctactggacggccggcggttgcstccgccgcgtggcctccgcgtccccgggccgccgcaggccgcgcccgaacgccccgatcggccgccgccgccgccgcgccgtccgcctccgcatccgccagggaggtgggcggggagcgttgactcgtcggcggtcgtaggggcggcgggtggcscggkgttgtccgnchmctttcgttcgcacggggccgggggcttccttcccgcgtgacgccgtgacgtgatgccgcgcgcccgggccccaagcctcagtgacgggcacgggccggccggttcgcggtggggaccccgcgcctcgtcgcccttttcacacctactggtcctctccttttggtttgttagattctgttttctatataccattttaggattcttttggtggccaaaaaagatgctgccggcctgctgcgagcgagagttcagtttggtaggccagtcaaaaagtggtagtcgtactagagtagtagtagtagtagactggtagtgctgctgctgaatgctgatgcaaaattcggcagcggtgaattggtgcctggggtcggtcaggtcagttggcagatttcagccaagccatggtttgttg

SEQ ID NO: 147 - DNA, 1070 bp, Zea mays
tccctctgtcggcagtctgcagcaggccagcgccgtcgcgcatgcaggaggcgtcatcggcggcggcgggggccgagcccggccgtcgggcggcgcagcatcagttcgccggcgtggacctccggcggcccaaggggtacgcgccgccggcgccggcggtgagcgagggggacccgtgcccgcggtgtgcgtcgcgggacaccaagttctgctactacaacaactacaacacctcccagccgcgccacttctgcaagggctgccgccgctactggaccaagggcggcacgctgcgcaacgtccccgtcggcggcggcacccgcaagaagccctccttgtcgtcgtcgtcgtcgtcgtcctacgcggccgccgcggacgccgacaggcagcccaagaagaagcccgccagcaagaagcgccgcgtcgtggcgccggccccggagctcgccgccgcggccgacccaggcaagacggcacccaccacgacgacgacgacgacgacgacgagcgagatcaccacggagactggcgcgctggaggactccgactccctggcgcacctgctgctgcagcccgggacagaggacgcggaggccgtcgcgctcgggctcggcctctccgacttcccctccgccgggaaggcggtgctggacgacgaggactcgttcgtgtggcccgccgcgtcgttcgacatgggcgcgtgctgggccggcgcagggttcgccgacccggaccccgcatgcatcttcctcaacctcccgtgacagccacagacggtcacggcgccgaggcgtacgtcctcctgctttgctctgctctgcgcctgatgatgagtgcaaggaaatggagcgctttgattcttctgctattggttgcatctgctaattgatttagcagtggtggtaaggtacacgcacgacgagctgactgcgtggttcttggcttgggccttagtatatgatgatgatgatgatactagttgtatgtgtgacgtgagagacgatcgcgacgacgaccttgaattttagtttctgagatgagaaaggtcaactagtggcctctgtatgact

SEQ ID NO: 148 - DNA, 1538 bp, Zea mays
cctccgccttccttgtgttccgcgcttccacaacagtttaccgcaaggaaggaaccatggacatgaactccaacgccaacaacagcactgccgcagcagcatcggctcccatcaacaaccagcaggaggctgtggtgtcatccccaaccagaaaggagcaagccaggaaccccaagaaggcgcgggcggcgccgcagcaggcgggcggcagcggggagcctaggccgcggcctccgccggacgcggcgcacagctgcccgcgctgctcctccaccaacacaaagttctgctactacaacaactacaacctgacgcagccgcgctacttctgcaagacgtgccgccgctactggacacacggcggcaccctccgcaacgtccccgtcggcggcggctgccgcaggaacaagcgcgcctccagctcctcgtccccgtttccgggcccctccagcaccgccgccaccagcgccgcgatggagaagaccgtcagcacgcggctgatgctgatggcgaccagcaccatggcgatgccctccccgacggcaggcctgtttgttcccgatgacatgtccccggcattcacgccgacgacgggcggtagcggcttcgacgacctcgccggcatggacgagcagcaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgacgacggtggttatgcagcaggaccagccgccggagtgcagcagcagcagcagcagcagcagcagcagattaatggtggtgatcaccagaagcaggacgagaacaaagaggcggggaacggcaaaggcaacgacgacggcggcggcgggtcgtcgtcggtgtacagcttctggatgaacaccagcggcagcgacggggcagaggggtagtgcgccactgccagtgccagccacaggaggaggcgaaagtgctgctgcaaactccctcgcatcatctggtcggtgcaagtgcagcagacatccttcgtcgacgcaatcaaatcatcaaaaggtggcaacagggagtgaaagggggaagaagttccccatccagcgtagaggatagggtgcatgttcatgtttgatttatctgcattgtggtcggtcgtggttgcaagtcatcttgcccccccctttgttttggttcagtttgcatgcgttcgttcgtcagagttttacttgcagcagtctttggatcgccggcagagattagttaggtatttatgctttgttttcgtcagttcgttctggataatgtcttaataattcctcagtttatttctttgttaaaaaaaaaaaaaaaaaaaaaaaaaaaaa

SEQ ID NO: 149 - DNA, 1443 bp, Zea mays
atgcacgaggcagcatcggctcccatcaacaaccagcaggaggctgtggtgtcatccccaaccagaaaggagcaagccaggaaccccaagaaggcgcgggcggcgccgcagcaggcgggcggcagcggggagcctaggccgcggcctccgccggacgcggcgcacagctgcccgcgctgctcctccaccaacacaaagttctgctactacaacaactacaacctgacgcagccgcgctacttctgcaagacgtgccgccgctactggacacacggcggcaccctccgcaacgtccccgtcggcggcggctgccgcaggaacaagcgcgcctccagctcctcgtccccgtttccgggcccctccagcaccgccgccaccagcgccgcgatggagaagaccgtcagcacgcggctgatgctgatggcgaccagcaccatggcgatgccctccccgacggcaggcctgtttgttcccgatgacatgtccccggcattcacgccgacgacgggcggtagcggcttcgacgacctcgccggcatggacgagcagcaccagcagggcttcctgcccttctcgccgctgtccctgtccgaccaggcgccggagctggctcctggaggagggggtgacacgacgccgtctttcctggacatgctgacaggagggtatctcgatggcggcggctacggcggcatgagcggtggcagcgatgcgatggacatgccgttctcgctgcctgagatggggcccccgacaactgatccaatgccgtttcagctccagtggacgtcatcagagcttgacaactacatcaacgacgacggtggttatgcagcaggaccagccgccggagtgcagcagcagcagcagcagcagcagcagcagattaatggtggtgatcaccagaagcaggacgagaacaaagaggcggggaacggcaaaggcaacgacgacggcggcggcgggtcgtcgtcggtgtacagcttctggatgaacaccagcggcagcgacggggcagaggggtagtgcgccactgccagtgccagccacaggaggaggcgaaagtgctgctgcaaactccctcgcatcatctggtcggtgcaagtgcagcagacatccttcgtcgacgcaatcaaatcatcaaaaggtggcaacagggagtgaaagggggaagaagttcaccatccagcgtagaggatagggtgcatgttcatgtttgatttatctgcattgtggtcggtcgtggttgcaagtcatcttgcaccaccctttgttttggttcagtttgcatgcgttcgttcgtcagagttttacttgcagcagtctttggatcgccggcagagattagttaggtatctatgctttgttttcgtcagttcgttctggataatgtcttaataattcctcagtttatttctttgttaactaaaaaaaaaaaaaaaa

SEQ ID NO: 150 - DNA, 1158 bp, Zea mays; identical to SEQ ID NO: 26

SEQ ID NO: 151 - DNA, 1392 bp, Zea mays; identical to SEQ ID NO: 29

SEQ ID NO: 152 - DNA, 618 bp, Zea mays; identical to SEQ ID NO: 41

SEQ ID NO: 153 - DNA, 618 bp, Zea mays; identical to SEQ ID NO: 62

SEQ ID NO: 154 - protein, 240 aa, Zea mays; identical to SEQ ID NO: 9

SEQ ID NO: 155 - protein, 351 aa, Zea mays; identical to SEQ ID NO: 18

SEQ ID NO: 156 - protein, 341 aa, Zea mays
MHEAASAPINNQQEAVVSSPTRKEQARNPKKARAAPQQAGGSGEPRPRPPPDAAHSCPRCSSTNTKFCYYNNYNLTQPRYFCKTCRRYWTHGGTLRNVPVGGGCRRNKRASSSSSPFPGPSSTAATSAAMEKTVSTRLMLMATSTMAMPSPTAGLFVPDDMSPAFTPTTGGSGFDDLAGMDEQHQQGFLPFSPLSLSDQAPELAPGGGGDTTPSFLDMLTGGYLDGGGYGGMSGGSDAMDMPFSLPEMGPPTTDPMPFQLQWTSSELDNYINDDGGYAAGPAAGVQQQQQQQQQQINGGDHQKQDENKEAGNGKGNDDGGGGSSSVYSFWMNTSGSDGAEG

SEQ ID NO: 157 - protein, 385 aa, Zea mays; identical to SEQ ID NO: 27

SEQ ID NO: 158 - protein, 463 aa, Zea mays; identical to SEQ ID NO: 30

SEQ ID NO: 159 - protein, 205 aa, Zea mays; identical to SEQ ID NO: 42

SEQ ID NO: 160 - protein, 205 aa, Zea mays; identical to SEQ ID NO: 63

(19) United States
(12) Patent Application Publication
(10) Pub. No.: US 2010/0175150 A1
(43) Pub. Date: Jul. 8, 2010

(54) DOF (DNA BINDING WITH ONE FINGER) SEQUENCES AND METHODS OF USE

(75) Inventors: Rajeev Gupta, JOhnston, IA (US); Juan Liu, Johnston, IA (US); Kanwarpal S. Dhugga, Johnston, IA (US); Carl R. Simmons, Des Moines, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/725,747

(22) Filed: Mar. 17, 2010

Related U.S. Application Data
(63) Continuation of application No. 11/593,955, filed on Nov. 7, 2006.
(60) Provisional application No. 60/735,645, filed on Nov. 10, 2005.

(51) Int. Cl. A01H 5/00; C12N 15/82; C07K 14/415; C12N 15/29; C12N 15/63; C12N 5/04

(52) U.S. Cl. 800/287; 800/298; 800/320.1; 800/320.3; 800/320.2; 800/320; 800/312; 800/306; 800/322; 800/314; 800/278; 530/379; 800/290; 536/23.6; 435/320.1; 435/419

(57) ABSTRACT

Methods and compositions are provided to improve nitrogen use efficiency in plants or plant parts, increase carbon fixation in a plant or plant part, increase grain yield or biomass production of the plant, and/or increase the stress tolerance of the plant. The compositions and methods of the invention modulate these various phenotypes by modulating the level of at least one Dof (for DNA binding with one finger) polypeptide having a Dof domain or a biologically active variant or fragment of a Dof domain.

### CROSS REFERENCE

This utility application is a continuation of U.S. Utility application Ser. No. 11/593,955, filed Nov. 7, 2006, which claims the benefit of U.S. Provisional Application Ser. No. 60/735,645, filed Nov. 10, 2005, both of which are incorporated herein by reference.

### FIELD OF THE INVENTION

The present invention is drawn to the field of genetics and molecular biology. More particularly, the compositions and methods are directed to modulation of carbon fixation, improving nitrogen use, improving yield and improving stress tolerance in plants.

### BACKGROUND OF THE INVENTION

Grain yield improvements by conventional breeding have nearly reached a plateau in maize. It is natural then to explore some alternative, non-conventional approaches that could be employed to obtain further yield increases. Since the harvest index in maize has remained essentially unchanged during selection for grain yield over the last hundred or so years, the yield improvements have been realized from the increased total biomass production per unit land area (Sinclair, et al., (1998) Crop Science 38:638-643; Duvick, et al., (1999) Crop Science 39:1622-1630 and Tollenaar, et al., (1999) Crop Science 39:1597-1604). This increased total biomass has been achieved by increasing planting density, which has led to adaptive phenotypic alterations, such as a reduction in leaf angle and tassel size, the former to reduce shading of lower leaves and the latter perhaps to increase harvest index (Duvick, et al., (1999) Crop Science 39:1622-1630).

Carbon fixation and nitrogen assimilation are two of the key processes that limit biomass production (Sinclair, et al., (1975) Science 189:565-567; Bhatia, et al., (1976) Science 194:1418-1421; Dhugga, et al., (1989) Crop Science 29:1232-1239 and Sinclair, et al., (1998) Crop Science 38:638-643). The energetic cost of making protein using nitrate nitrogen, which is the main form of nitrogen acquired from the soil in maize, from a unit of photosynthate is approximately twice of that needed to make carbohydrates (Penning, et al., (1974) J. Theor. Biol. 45:339-377 and Sinclair, et al., (1975) Science 189:565-567). In agreement with this, protein concentration in the maize grain has gone down as a result of selection for grain yield at higher planting densities (Duvick, et al., (1999) Crop Science 39:1622-1630). Ideally, grain yield is maximized with minimal amount of applied nitrogen. Aside from increasing fertilizer prices, run-off and leached nitrate cause undesirable environmental effects (Frink, et al., (1999) PNAS 96:1175-1180). Whereas one of the open avenues is to select for reduced grain protein content that might be reflected in increased grain yield because of accumulation of a greater amount of carbohydrates, the other is to increase the rate of photosynthesis (Leakey, et al., (2004) Global Change Biology 10:951-962).

Given the complexity of the metabolic pathways, it is unlikely that single gene alterations will prove fruitful in improving biomass production (Morandini, et al., (2003) Trends in Plant Science 8:70-75). However, synchronous improvement in different components of a whole pathway might allow overcoming, at least to some extent, the complexity of the metabolic pathways. Upstream regulators of gene expression could help accomplish this goal (Morandini, et al., (2003) Trends in Plant Science 8:70-75). A single upstream ‘master-regulatory’ gene, for example, may be utilized to alter the expression of multiple metabolic genes in a pathway (Rabinowicz, et al., (1999) Genetics 153:427-444; DellaPenna (2001) Plant Physiol 125:160-153; Morandini, et al., (2003) Trends in Plant Science 8:70-75). These types of genes are referred to as transcription factors (TF). TF operate at a higher level of molecular hierarchy and play key roles in various biological processes. This is obvious from the fact that approximately 5% of Arabidopsis genome encodes TF (˜1500), which are classified into approximately 50 different groups based on the DNA-binding domains that are specific to each group (Riechmann, et al., (2000) Science 290:2105-2110).

Methods and compositions are needed in the art which can employ such master regulatory sequence to modulate carbon fixation and nitrogen assimilation in plants.

### BRIEF SUMMARY OF THE INVENTION

Compositions of the invention comprise isolated polypeptides comprising an amino acid sequence selected from the group consisting of the amino acid sequence comprising SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 or a variant or fragment thereof.

Compositions also comprise isolated polynucleotides comprising a nucleotide sequence selected from the group consisting of the nucleotide sequence comprising SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153 or a variant or fragment thereof.

Expression cassettes, plants, plant cells, plant parts and seeds comprising these sequences are further provided. In specific embodiment, the polynucleotide is operably linked to a tissue-preferred promoter including, but not limited to, a leaf-preferred promoter, a mesophyll-preferred promoter, a bundle sheath-preferred promoter, a vascular-preferred promoter, a seed-preferred promoter, an endosperm-preferred promoter or an embryo-preferred promoter.

Methods for modulating the level of a Dof polypeptide in a plant or a plant part are provided. The methods comprise introducing into a plant or plant part a heterologous polynucleotide comprising a Dof sequence of the invention. The level of the Dof polypeptide can be increased or decreased. Such method can be used to increase the yield in plants, increase the nitrogen use efficiency of a plant and/or improve the stress response of the plant.

Further compositions of the invention comprise isolated expression cassettes comprising a polynucleotide operably linked to a leaf-preferred promoter or a vascular-preferred promoter, wherein the polynucleotide is selected from the group consisting of (a) a polynucleotide encoding a Dof polypeptide comprising an amino acid sequence set forth in SEQ ID NO: 145; (b) a polynucleotide encoding a Dof polypeptide comprising an amino acid sequence having at least 80% sequence identity to SEQ ID NO: 145, wherein the Dof polypeptide is capable of modulating transcription; (c) a polynucleotide which when expressed in a plant decrease the expression level of a Dof polypeptide comprising an amino acid sequence set forth in SEQ ID NO: 145 and (d) a polynucleotide which when expressed in a plant decrease the expression level of a Dof polypeptide comprising an amino acid sequence having at least 80% sequence identity to SEQ ID NO: 145, wherein the Dof polypeptide is capable of modulating transcription. Plants, plant parts, cells and seeds having this expression cassette are also provided.

Further provide are methods for increasing nitrogen efficiency in a plant, increasing yield in a plant and improving the stress response of a plant. Such methods comprise introducing into the plant a heterologous polynucleotide and expressing the polynucleotide in the plant from an operably linked leaf-preferred promoter or a vascular preferred promoter. In such methods, the expression of the heterologous polynucleotide modulates the level of at least one Dof polypeptide comprising an amino acid sequence set forth in SEQ ID NO: 145 or a biologically active variant or fragment thereof.

### BRIEF DESCRIPTION OF THE DRAWINGS

FIG. 1 provides an alignment of characterized Dof domains from various Dof polypeptides from rice.

FIGS. 2A and 2B provide an alignment of the Dof domain from the various members of the maize Dof family. Conserved regions are highlighted. The consensus Dof domain (SEQ ID NO: 145) is set forth above the alignment.

### DETAILED DESCRIPTION OF THE INVENTION

The present invention will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which this invention pertains having the benefit of the teachings presented in the foregoing descriptions. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions are provided to improve nitrogen use efficiency in plants or plant parts, increase carbon fixation in a plant or plant part, increase yield or biomass production of the plant and/or increase the stress tolerance of the plant. The compositions and methods of the invention modulate these various phenotypes by modulating in a plant the level of at least one Dof polypeptide having a Dof domain or a polypeptide having a biologically active variant or fragment of a Dof domain.

### II. Compositions

A. Dof Polynucleotides and Polypeptides

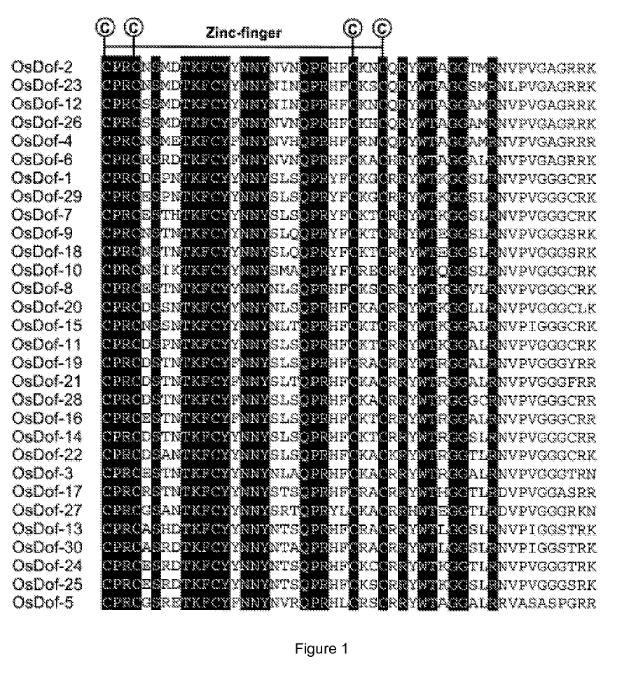

Compositions of the invention include Dof polynucleotides and polypeptides and variants and fragments thereof that are involved in regulating transcription. Dof (for DNA binding with one finger) is a family of DNA binding proteins that have been found in diverse plant species. Members of the Dof family comprise a Dof domain or an active variant or fragment thereof, which is a highly conserved amino acid sequence involved in DNA binding. The Dof domain is characterized by a conserved region of about 50 amino acids with a C2-C2 finger structure, associated with a basic region. The basic region of specific members of the Dof family can bind to DNA sequences with a 5′-T/AAAAG-3′ core. See, for example, Lijavetzky, et al., (2003) BMC Evolutionary Biology 3:17 and Yanagisawa, et al., (1999) Plant J. 17:209. FIG. 1 provides a sequence alignment of Dof domains from several characterized Dof polypeptides. The consensus sequence for the Dof domain is set forth in SEQ ID NO: 145.

As used herein, a “Dof” sequence comprises a polynucleotide encoding or a polypeptide having the conserved Dof domain or a biologically active variant or fragment of the Dof domain. The consensus Dof domain is as follows: C-P-R-C-X-S-X-[DHN]-T-K-F-C-Y-[FY]-N-N-Y-[NS]-X-X-Q-P-R-[HY]-[FL]-C-[KR]-X-C-[RKQH]-R-[YH]-W-T-X-G-G-[TASV]-[LMI]-R (shaded residues are highly conserved among Dof members, X represents any amino acid, and [ ] surrounds the recited amino acids that can be found in that position). SEQ ID NO: 145 sets forth this conserved domain. It is recognized, however, that the conserved sequences set forth in the Dof domain consensus sequence can be altered and still retain Dof activity (i.e., the ability to modulate transcription). See, for example, Yanagisawa, et al., (2001) Plant Cell Physiol. 42:813-22 and Lijavetzky, et al., (2003) BMC Evolutionary Biology 3:17 and FIG. 1. Table 2 also provides representative Dof domains from various maize Dof polypeptides. Biologically active fragments and variants of a Dof domain will continue to retain the ability to modulate transcription when the domain is placed within the context of an appropriate polypeptide.

In one embodiment, the present invention provides isolated Dof polypeptides comprising amino acid sequences as shown in SEQ ID NOS: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160. Further provided are polynucleotides comprising the nucleotide sequence set forth in SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153. The conserved Dof domains in SEQ ID NOS: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 are outlined in Table 1.

The invention encompasses isolated or substantially purified polynucleotide or protein compositions. An “isolated” or “purified” polynucleotide or protein or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

Fragments and variants of the Dof domain or Dof polynucleotides and proteins encoded thereby are also encompassed by the methods and compositions of the present invention. By “fragment” is intended a portion of the polynucleotide or a portion of the amino acid sequence. Fragments of a polynucleotide may encode protein fragments that retain the biological activity of the native protein and hence regulate transcription. Alternatively, fragments that are used for suppressing or silencing (i.e., decreasing the level of expression) of a Dof sequence need not encode a protein fragment, but will retain the ability to suppress expression of the target Dof sequence. In addition, fragments that are useful as hybridization probes generally do not encode fragment proteins retaining biological activity. Thus, fragments of a nucleotide sequence may range from at least about 18 nucleotides, about 20 nucleotides, about 50 nucleotides, about 100 nucleotides and up to the full-length polynucleotide encoding the proteins of the invention.

A fragment of a polynucleotide encoding a Dof domain or a Dof polypeptide will encode at least 15, 25, 30, 50, 100, 150, 200, 250, 275, 300, 352, 350, 375, 400, 425, 450, 475, 480 contiguous amino acids or up to the total number of amino acids present in a full-length Dof domain or Dof protein (i.e., SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160). Fragments of a Dof domain or a Dof polynucleotide that are useful as hybridization probes, PCR primers or as suppression constructs generally need not encode a biologically active portion of a Dof protein or a Dof domain.

A biologically active portion of a Dof domain or a Dof protein can be prepared by isolating a portion of a Dof polynucleotide, expressing the encoded portion of the Dof protein (e.g., by recombinant expression in vitro) and assessing the activity of the encoded portion of the Dof protein. Polynucleotides that are fragments of a Dof domain or a Dof nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, 2,000, 2,050, 2,080 contiguous nucleotides or up to the number of nucleotides present in a full-length Dof domain or in a Dof polynucleotide (i.e., SEQ ID NOS: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153).

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the Dof polypeptides or of a Dof domain. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode a Dof domain or a Dof polypeptide that is capable of regulating transcription or that is capable of reducing the level of expression (i.e., suppressing or silencing) of a Dof polynucleotide. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%,99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, that is, regulate transcription as described herein. Such variants may result from, for example, genetic polymorphism or from human manipulation. Biologically active variants of a Dof protein of the invention or of a Dof domain will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the Dof protein or the consensus Dof domain as determined by sequence alignment programs and parameters described elsewhere herein. A biologically active variant of a Dof protein of the invention or of a Dof domain may differ from that protein by as few as 1-15 amino acid residues, as few as 1-10, such as 6-10, as few as 5, as few as 4, 3, 2 or even 1 amino acid residue.

The polynucleotides of the invention may be altered in various ways including amino acid substitutions, deletions, truncations and insertions. Methods for such manipulations are generally known in the art. For example, amino acid sequence variants and fragments of the Dof proteins or Dof domains can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired activity (i.e., the ability to regulate transcription or decrease the level of expression of a target Dof sequence). In specific embodiments, the mutations that will be made in the DNA encoding the variant does not place the sequence out of reading frame and does not create complementary regions that could produce secondary mRNA structure. See, EP Patent Application Publication Number 75,444.

The deletions, insertions and substitutions of the protein sequences encompassed herein are not expected to produce radical changes in the characteristics of the protein. However, when it is difficult to predict the exact effect of the substitution, deletion or insertion in advance of doing so, one skilled in the art will appreciate that the effect will be evaluated by routine screening assays. For example, the activity of a Dof polypeptide can be evaluated by assaying for the ability of the polypeptide to regulate transcription. Various methods can be used to assay for this activity, including, directly monitoring the level of expression of a target gene at the nucleotide or polypeptide level. Methods for such an analysis are known and include, for example, Northern blots, S1 protection assays, Western blots, enzymatic or colorimetric assays. In specific embodiments, determining if a sequence has Dof activity can be assayed by monitoring for an increase or decrease in the level or activity of target genes, including various enzymes in the carbon fixation and nitrogen assimilation pathways. For example, in specific embodiments, a Dof sequence can modulate transcription of target genes such as the phosphoenolpyruvate carboxylase gene, the cytoplasmic pyruvate ortho-phosphate dikinase gene, nitrate reductase, glutamine synthase, glutamate synthase, glutamate dehydrogenase, isocitrate dehydrogenase and asparagines synthase. See, for example, Yanagisawa, et al., (2002) Trends in Plant Science 7:555-560 and Yanagisawa, et al., (2000) Plant J. 21:281-288, both of which are herein incorporated by reference. Alternatively, methods to assay for a modulation of transcriptional activity can include monitoring for an alteration in the phenotype of the plant. For example, as discussed in further detail elsewhere herein, modulating the level of a Dof polypeptide can result in increased carbon fixation, improved nitrogen use efficiency and grain yield and improved tolerance of the plant to environmental stress, including abiotic stresses such as drought, heat and nitrogen stress. Methods to assay for these changes are discussed in further detail elsewhere herein.

Variant polynucleotides and proteins also encompass sequences and proteins derived from a mutagenic and recombinogenic procedure such as DNA shuffling. With such a procedure, one or more different Dof coding sequences can be manipulated to create a new Dof sequence or Dof domain possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the Dof gene of the invention and other known Dof genes to obtain a new gene coding for a protein with an improved property of interest, such as an increased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458.

The polynucleotides of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire DOF sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99% or greater sequence identity. Functions of orthologs are often highly conserved among species. Thus, isolated polynucleotides that can silence or suppress the expression of a Dof sequence or a polynucleotide that encodes for a Dof protein and which hybridize under stringent conditions to the Dof sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, N.Y.). See also, Innis, et al., eds. (1990) PCR Protocols: A Guide to Methods and Applications (Academic Press, New York); Innis and Gelfand, eds. (1995) PCR Strategies (Academic Press, New York) and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments or other oligonucleotides and may be labeled with a detectable group such as ³²P or any other detectable marker. Thus, for example, probes for hybridization can be made by labeling synthetic oligonucleotides based on the DOF polynucleotides of the invention. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.).

For example, the entire Dof polynucleotide or a polynucleotide encoding a Dof domain disclosed herein, or one or more portions thereof, may be used as a probe capable of specifically hybridizing to corresponding Dof polynucleotide and messenger RNAs. To achieve specific hybridization under a variety of conditions, such probes include sequences that are unique among Dof polynucleotide sequences and are optimally at least about 10 nucleotides in length and most optimally at least about 20 nucleotides in length. Such probes may be used to amplify corresponding Dof polynucleotide from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York). See, Sambrook, et al., (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller (1988) supra. A PAM120 weight residue table, a gap length penalty of 12 and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2 and the BLOSUM62 scoring matrix or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

B. Plants

In specific embodiments, the invention provides plants, plant cells and plant parts having altered levels (i.e., an increase or decrease) of a Dof sequence. In some embodiments, the plants and plant parts have stably incorporated into their genome at least one heterologous polynucleotide encoding a Dof polypeptide comprising the Dof domain as set forth in SEQ ID NO: 145 or a biologically active variant or fragment thereof. In one embodiment, the polynucleotide encoding the Dof polypeptide is set forth in any one of SEQ ID NOS: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152, 153 or a biologically active variant or fragment thereof.

In yet other embodiments, plants and plant parts are provided in which the heterologous polynucleotide stably integrated into the genome of the plant or plant part comprises a polynucleotide which when expressed in a plant decreases the level of a Dof polypeptide comprising a Dof domain as set forth in SEQ ID NO: 145 or an active variant or fragment thereof. Sequences that can be used to suppress expression of a Dof polypeptide include, but are not limited to, any of the sequence set forth in SEQ ID NOS: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 144, 146, 147, 148, 149, 150, 151, 152 or 153 or variants or fragments thereof.

In specific embodiments, the heterologous polynucleotide in the plant or plant part is operably linked to a tissue-preferred promoter, such as a seed-preferred promoter (i.e., an endosperm-preferred promoter or an embryo-preferred promoter), a vascular-preferred promoter or a leaf-preferred promoter (i.e., a bundle sheath-preferred promoter or a mesophyll-preferred promoter).

As discussed in further detail elsewhere herein, such plants, plant cells and plant parts can have an altered phenotype including, for example, a modulation in carbon fixation, improved nitrogen use efficiency, improved yield or an improved stress tolerance.

As used herein, the term plant includes plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the invention, provided that these parts comprise the introduced or heterologous polynucleotides disclosed herein.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus effiotii), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which an alteration, such as transformation or introduction of a polypeptide, has occurred, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed.

C. Polynucleotide Constructs

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures and the like.

The various polynucleotides employed in the methods and compositions of the invention can be provided in expression cassettes for expression in the plant of interest. The cassette will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the DOF polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a Dof polynucleotide and a transcriptional and translational termination region (i.e., termination region) functional in plants. The regulatory regions (i.e., promoters, transcriptional regulatory regions and translational termination regions) and/or the Dof polynucleotide may be native/analogous to the host cell or to each other. Alternatively, the regulatory regions and/or the Dof polynucleotides may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of Dof in the plant or plant cell. Thus, the phenotype of the plant or plant cell can be altered.

The termination region may be native with the transcriptional initiation region, may be native with the operably linked Dof polynucleotide of interest, may be native with the plant host or may be derived from another source (i.e., foreign or heterologous) to the promoter, the Dof polynucleotide of interest, the plant host or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20) and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

Tissue-preferred promoters can be utilized to target enhanced expression within a particular plant tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Promoters that direct expression in various types of leaf cells can also be employed. For example, mesophyll-preferred promoters are known in the art and include, but are not limited to, the promoter for the C₄ phosphoenolpyruvate carboxylase gene (Gowik, et al., (2004) The Plant Cell 16:1077-1090); the promoter of the chlorophyll a/b binding protein gene (cab) (Hudspeth, et al., (1992) Plant Physiology 98:458-464); the Arabidopsis promoter pRbcS2b (Moon, et al., “A novel screening approach for selective non-cell autonomous proteins” American Society of Plant Biologists Abs #864); the promoters of the cytosolic fructose-1,6-bisphosphatase genes (cy-FBPase genes) (US Patent Application Publication Number 2002/120955) and the ribulose-1,5-bisphosphate carboxylase small subunit (rbcS) gene promoters from rice and maize (Schaffner, et al., (1991) The Plant Cell 9:997-1012), each of these references is herein incorporated by reference.

Other leaf-preferred promoters of interest include bundle sheath-preferred promoters. Bundle sheath-preferred promoters are known in the art and include, but are not limited to, a modified form of the promoter from ppcA (Stockhaus, (1997) Plant Cell 9:479) and the ZjPck promoter which directs expression in bundle sheath cells and in vascular cells (Nomura, (2005) Plant and Cell Physiology 46(5):754-761, each of these references is herein incorporated by reference.

Vascular-preferred promoters are also known in the art including, but not limited to, promoters of US Patent Application Publication Number 2004/0163146.

Various promoters that are induced by light can be used in the methods and compositions of the invention. Such promoter as known in the art and include, but are not limited to, the promoters from cab or rubisco (Simpson, et al., (1985) EMBO J 4:2723-2729 and Timko, et al., (1985) Nature 318:579-582).

Seed-preferred promoters include both seed-specific promoters (those promoters active during seed development such as promoters of seed storage proteins), as well as, seed-germinating promoters (those promoters active during seed germination). See, Thompson, et al., (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see, WO 00/11177 and U.S. Pat. No. 6,225,529, herein incorporated by reference), PCNA2 (U.S. patent application Ser. No. 10/388,359, filed Mar. 13, 2003) and CKX1-2 (US Patent Application Publication Number 2002/0152500). For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also, WO 00/12733, where seed-preferred promoters from end1 and end2 genes are disclosed and WO 01/21783 and U.S. Pat. No. 6,403,862, where the Zm40 promoter is disclosed, both herein incorporated by reference.

Embryo-specific promoters include Globulin 1 (Glb-1), ESR (US Patent Application Publication Number 2004/0210960) and lecI (U.S. patent application Ser. No. 09/718,754, filed Nov. 22, 2000). Additional embryo specific promoters are disclosed in Sato, et al., (1996) Proc. Natl. Acad. Sci. 93:8117-8122; Nakase, et al., (1997) Plant J 12:235-56 and Postma-Haarsma, et al., (1999) Plant Mol. Biol. 39:257-71. Endosperm-preferred promoters include the Gamma-zein, promoter, eppI and eep2 as disclosed in US Patent Application Publication Number 2004/0237147. Additional endosperm-specific promoters are disclosed in Albani, et al., (1985) EMBO 3:1505-15; Albani, et al., (1999) Theor. Appl. Gen. 98:1253-62; Albani, et al., (1993) Plant J. 5:353-55; Mena, et al., (1998) The Plant Journal 116:53-62 and Wu, et al., (1998) Plant Cell Physiology 39:885-889. Immature ear tissue-preferred promoters can also be employed.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Ad. USA 86:5400-5404; Fuerst, et al., (1989) Proc. Natl. Acad. Sci. USA 86:2549-2553; Deuschle, et al., (1990) Science 248:480-483; Gossen (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

In certain embodiments the polynucleotides of the present invention can be stacked with any combination of polynucleotide sequences of interest in order to create plants with a desired trait. A trait, as used herein, refers to the phenotype derived from a particular sequence or groups of sequences. The combinations generated can also include multiple copies of any one of the polynucleotides of interest. The polynucleotides of the present invention can also be stacked with traits desirable for disease or herbicide resistance (e.g., fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides providing agronomic traits such as male sterility (e.g., see, U.S. Pat. No. 5,583,210), stalk strength, flowering time or transformation technology traits such as cell cycle regulation or gene targeting (e.g., WO 99/61619, WO 00/17364 and WO 99/25821); the disclosures of which are herein incorporated by reference.

These stacked combinations can be created by any method including, but not limited to, cross-breeding plants by any conventional or TopCross methodology or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 99/25821, WO 99/25854, WO 99/25840, WO 99/25855 and WO 99/25853, all of which are herein incorporated by reference.

D. Method of Introducing

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722) and ballistic particle acceleration (see, for example, U.S. Pat. No. 4,945,050; U.S. Pat. No. 5,879,918; U.S. Pat. Nos. 5,886,244 and 5,932,782; Tomes, et al., (1995) in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

In specific embodiments, the Dof sequences or variants and fragments thereof can be provided to a plant using a variety of transient transformation methods. Such transient transformation methods include, but are not limited to, the introduction of the Dof protein or variants and fragments thereof directly into the plant or the introduction of the Dof transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, the Dof polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the a Dof sequence or a variant or fragment thereof may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 99/25821, WO 99/25854, WO 99/25840, WO 99/25855 and WO 99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown and either pollinated with the same transformed strain or different strains and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

### III. Methods of Use

A. Methods for Modulating Expression of at Least One Dof Sequence or a Variant or Fragment Therefore in a Plant or Plant Part

A “modulated level” or “modulating level” of a polypeptide in the context of the methods of the present invention refers to any increase or decrease in the expression, concentration or activity of a gene product, including any relative increment in expression, concentration or activity. Any method or composition that modulates expression of a target gene product, either at the level of transcription or translation or modulates the activity of the target gene product can be used to achieve modulated expression, concentration, activity of the target gene product. In general, the level is increased or decreased by at least 1%, 5%, 10%, 20%, 30%, 40%, 50%, 60%, 70%, 80%, 90% or greater relative to an appropriate control plant, plant part, or cell. Modulation in the present invention may occur during and/or subsequent to growth of the plant to the desired stage of development. In specific embodiments, the polypeptides of the present invention are modulated in monocots, particularly maize.

The expression level of a polypeptide having a Dof domain or a biologically active variant or fragment thereof may be measured directly, for example, by assaying for the level of the Dof polypeptide in the plant, or indirectly, for example, by measuring the level of the polynucleotide encoding the protein or by measuring the activity of the Dof polypeptide in the plant. Methods for determining the activity of the Dof polypeptide are described elsewhere herein.

In specific embodiments, the polypeptide or the polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984, all of which are herein incorporated by reference. See also, WO 98/49350, WO 99/07865, WO 99/25821 and Beetham, et al., (1999) Proc. Natl. Acad. Sci. USA 96:8774-8778, herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions and substitutions of nucleotides into the genome. While the methods of the present invention do not depend on additions, deletions and substitutions of any particular number of nucleotides, it is recognized that such additions, deletions or substitutions comprises at least one nucleotide.

In one embodiment, the activity and/or level of a Dof polypeptide is increased. An increase in the level and/or activity of the Dof polypeptide can be achieved by providing to the plant a Dof polypeptide or a biologically active variant or fragment thereof. As discussed elsewhere herein, many methods are known in the art for providing a polypeptide to a plant including, but not limited to, direct introduction of the Dof polypeptide into the plant or introducing into the plant (transiently or stably) a polynucleotide construct encoding a polypeptide having Dof activity. It is also recognized that the methods of the invention may employ a polynucleotide that is not capable of directing in the transformed plant the expression of a protein or an RNA. Thus, the level and/or activity of a Dof polypeptide may be increased by altering the gene encoding the Dof polypeptide or its promoter. See, e.g., Kmiec, U.S. Pat. No. 5,565,350; Zarling, et al., PCT/US93/03868. Therefore mutagenized plants that carry mutations in Dof genes, where the mutations increase expression of the Dof gene or increase the activity of the encoded Dof polypeptide are provided.

In other embodiments, the activity and/or level of the Dof polypeptide of the invention is reduced or eliminated by introducing into a plant a polynucleotide that inhibits the level or activity of a polypeptide. The polynucleotide may inhibit the expression of Dof directly, by preventing translation of the Dof messenger RNA, or indirectly, by encoding a polypeptide that inhibits the transcription or translation of a Dof gene encoding a Dof protein. Methods for inhibiting or eliminating the expression of a gene in a plant are well known in the art and any such method may be used in the present invention to inhibit the expression of at least one Dof sequence in a plant. In other embodiments of the invention, the activity of a Dof polypeptide is reduced or eliminated by transforming a plant cell with a sequence encoding a polypeptide that inhibits the activity of the Dof polypeptide. In other embodiments, the activity of a Dof polypeptide may be reduced or eliminated by disrupting the gene encoding the Dof polypeptide. The invention encompasses mutagenized plants that carry mutations in Dof genes, where the mutations reduce expression of the Dof gene or inhibit the Dof activity of the encoded Dof polypeptide.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy, et al., (1988) Proc. Natl. Acad. Sci. USA 85:8805-8809 and U.S. Pat. Nos. 5,107,065; 5,453,566 and 5,759,829); cosuppression (e.g., Taylor, (1997) Plant Cell 9:1245; Jorgensen, (1990) Trends Biotech. 8(12):340-344; Flavell, (1994) Proc. Natl. Acad. Sci. USA 91:3490-3496; Finnegan, et al., (1994) Bio/Technology 12:883-888 and Neuhuber, et al., (1994) Mol. Gen. Genet. 244:230-241); RNA interference (Napoli, et al., (1990) Plant Cell 2:279-289; U.S. Pat. No. 5,034,323; Sharp, (1999) Genes Dev. 13:139-141; Zamore, et al., (2000) Cell 101:25-33 and Montgomery, et al., (1998) Proc. Natl. Acad. Sci. USA 95:15502-15507), virus-induced gene silencing (Burton, et al., (2000) Plant Cell 12:691-705 and Baulcombe, (1999) Curr. Op. Plant Bio. 2:109-113); target-RNA-specific ribozymes (Haseloff, et al., (1988) Nature 334:585-591); hairpin structures (Smith, et al., (2000) Nature 407:319-320; WO 99/53050; WO 02/00904; WO 98/53083; Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7, US Patent Application Publication Number 2003/0175965; Panstruga, et al., (2003) Mol. Biol. Rep. 30:135-140; Wesley, et al., (2001) Plant J. 27:581-590; Wang and Waterhouse, (2001) Curr. Opin. Plant Biol. 5:146-150; US Patent Application Publication Number 2003/0180945 and WO 02/00904, all of which are herein incorporated by reference); ribozymes (Steinecke, et al., (1992) EMBO J. 11:1525 and Perriman, et al., (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345 and WO 00/42219); transposon tagging (Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928; Bensen, et al., (1995) Plant Cell 7:75-84; Mena, et al., (1996) Science 274:1537-1540 and U.S. Pat. No. 5,962,764), each of which is herein incorporated by reference and other methods or combinations of the above methods known to those of skill in the art.

It is recognized that with the polynucleotides of the invention, antisense constructions, complementary to at least a portion of the messenger RNA (mRNA) for the Dof sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used.

The polynucleotides of the present invention may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

Thus, many methods may be used to reduce or eliminate the activity of a Dof polypeptide or a biologically active variant or fragment thereof. In addition, combinations of methods may be employed to reduce or eliminate the activity of at least one Dof polypeptide. It is further recognized that the level of a single Dof sequence can be modulated to produce the desired phenotype. Alternatively, is may be desirable to modulate (increase and/or decrease) the level of expression of multiple sequences having a Dof domain or a biologically active variant or fragment thereof. To decrease the level of a single Dof sequence (or highly related Dof sequences) suppression constructs can be employed that target the suppression of a specific Dof sequence or a specific subset of Dof sequences. Alternatively, if it is desirable to suppress a wide range of Dof sequences, the suppression constructs could employ sequences that are highly conserved among Dof family members, such as the Dof domain.

As discussed above, a variety of promoters can be employed to modulate the level of the Dof sequence. In one embodiment, the expression of the heterologous polynucleotide which modulates the level of at least one Dof polypeptide can be regulated by a tissue-preferred promoter, particularly, a leaf-preferred promoter (i.e., mesophyll-preferred promoter or a bundle sheath preferred promoter) and/or a seed-preferred promoter (i.e., an endosperm-preferred promoter or an embryo-preferred promoter).

B. Methods to Modulate Carbon Fixation, Nitrogen Assimilation, Yield and/or Stress Tolerance in a Plant

Nitrogen assimilation is essential to the growth and development of plants, and therefore, large quantities of nitrogen fertilizers are used on plants to maximize crop yields. Such nitrogen fertilizers, however, aside from constituting the single most expense farm input, have negative impacts on the environment. Accordingly, methods and compositions are provided to increase the ability of a plant or plant part to assimilate nitrogen and thereby improve plant yields. Such methods comprise modulating the level of at least one Dof polynucleotide having a Dof domain in a plant or plant part and thereby increasing nitrogen assimilation (increased nitrogen use efficiency) and/or plant yield.

An increase in nitrogen assimilation can be assayed by determining the nitrogen content of the plant or plant part. For example, increasing the level of nitrogen assimilation can comprise an increase in overall nitrogen content of the plant or plant part of about 0.1%, 0.5%, 1%, 3% 5%, 10%, 15%, 20%, 25%, 30%, 35%, 40%, 45%, 50%, 55%, 60%, 65%, 70%, 75%, 80%, 85%, 90%, 95% or greater when compared to a control plant or plant part. Alternatively, the increased level of the nitrogen content can include about a 0.5 fold, 1 fold, 2 fold, 4 fold, 8 fold, 16 fold or 32 fold increase in overall increase in nitrogen level in the plant or a plant part when compared to a control plant or plant part. Methods to assay for the level of nitrogen are known. See, for example, Yanagisawa, et al., (2004) PNAS 101:7833-7838 and Stitt, et al., (1989) Methods Enzymol. 174:518-552, both of which are herein incorporated by reference in their entirety.

An increase in nitrogen assimilation can also be assayed by determining the level of amino acids in a plant or plant part. “Increasing the level of an amino acid” includes any increase in amino acid level in the plant or plant part. For example, increasing the level of an amino acid can comprise an increase in overall amino acid content of the plant or plant part of about 0.1%, 0.5%, 1%, 3% 5%, 10%, 15%, 20%, 25%, 30%, 35%, 40%, 45%, 50%, 55%, 60%, 65%, 70%, 75%, 80%, 85%, 90%, 95% or greater when compared to a control plant or plant part. Alternatively, the increased level of the amino acid can include about a 0.5 fold, 1 fold, 2 fold, 4 fold, 8 fold, 16 fold or 32 fold increase in overall increase in amino acid level in the plant or a plant part when compared to a control plant or plant part.

It is further recognized that the increase in the level of an amino acid need not be an overall increase in amino acid level, but also includes a change in the level of a single amino acid or a combination of amino acids. In this embodiment, the increase in amino acid level need not be an overall increase in amino acid concentration, but also includes a change in the ratio of various amino acids. For example, an increase in amino acid content could be reflected through an elevated level of glutamine or glutamate, which are good markers for nitrogen utilization. See, for example, Stitt, et al., (1999) Plant Cell Environ. 22:583-621, Matt, et al., (2002) Plant J. 30:663-677 and Foyer, et al., (2003) J. Exp. Bot. 54:585-593 and Yanagisawa, et al., (2004) PNAS 101:7833-7838, all of which are herein incorporated by reference.

An increase in nitrogen assimilation (increase in nitrogen use efficiency) can also be assayed by monitoring the tolerance of the plant to nitrogen stress. Such assays are discussed in further detail elsewhere herein. Briefly, a modulation in nitrogen assimilation can be assayed by determining if the plant or plant part displays better growth under low nitrogen conditions when compared to a control plant or plant part. Such a phenotype could comprise the lack of leaf discoloration under low nitrogen growth conditions. See, for example, Yanagisawa, et al., (2004) Proc. Natl. Acad. Sci 101:7833-7838, herein incorporated by reference.

The methods and compositions further can be used to increase yield in a plant. As used herein, the term “improved yield” means any improvement in the yield of any measured plant product. The improvement in yield can comprise a 0.1%, 0.5%, 1%, 3%, 5%, 10%, 15%, 20%, 30%, 40%, 50%, 60%, 70%, 80%, 90% or greater increase in measured plant product. Alternatively, the increased plant yield can comprise about a 0.5 fold, 1 fold, 2 fold, 4 fold, 8 fold, 16 fold or 32 fold increase in measured plant products. For example, an increase in the bu/acre yield of soybeans or corn derived from a crop having the present treatment as compared with the bu/acre yield from untreated soybeans or corn cultivated under the same conditions would be considered an improved yield.

Methods are also provided to improve stress tolerance of a plant. The methods of the invention comprise modulating the level of a polypeptide having a Dof domain in a plant or plant part and thereby increasing the stress tolerance of a plant.

As used herein, abiotic stress tolerance includes, but is not limited to, increased yield, growth, biomass, health or other measure that, when compared to an appropriate control plant, indicates tolerance to a stress which includes, but is not limited to, heat stress, salt stress, cold stress (including cold stress during germination), heat stress, water stress (including but not limited to drought stress) and nitrogen stress (including high and low nitrogen).

“Heat tolerance” is defined herein as a measure of the ability of the plant to grow under conditions where heat or warmer temperature would detrimentally affect the growth, vigor, yield and/or size, of an appropriate control plant. Plants exhibiting an improved heat tolerance grow better under conditions of heat stress than non-heat tolerant plants.

“Cold tolerance” is defined herein as a measure of the ability of a plant to grow under conditions where cold or cooler temperature would detrimentally affect the growth, vigor, yield and/or size of an appropriate control plant. Plants exhibiting an improved cold tolerance grow better under conditions of cold stress than non-cold tolerant plants.

“Drought” as defined herein refers to a period of dryness that, especially when prolonged, can cause damage to crops or prevent their successful growth (i.e., decreased vigor, growth, size, root length and/or and various other physiologic and physical measures). Plants exhibiting an improved drought tolerance grow better under conditions of drought stress than non-drought tolerant plants.

“Nitrogen stress” is defined herein as either an increase or decrease in the presence of nitrogen that can cause damage to crops or prevent their successful growth (i.e., decreased vigor, growth, size, root length and/or and various other physiologic and physical measures). Plants that exhibit an improved tolerance to nitrogen stress grow better under conditions of low and/or high nitrogen stress than the appropriate control plants from the same species. Methods to assay for improved tolerance to nitrogen stress are known. See, for example, Sepehri, et al., (2003) Journal of Biol. Sciences 3:578-584; Henry, et al., (1992) Int J Plant Sci 153:178-85; Banzinger, et al., (2000) Breeding for Drought and Nitrogen Stress Tolerance in Maize: From Theory to Practice. Mexico, D.F.:CIMMYT; Dhugga and Waines, (1989) Crop Science 29:1232 and Sicher, et al., (2005) Pysiologia Plantarum 123:219, each of which is herein incorporated by reference.

Accordingly, various methods to increase nitrogen assimilation, increase yield and/or increase the stress tolerance of a plant are provided. In one embodiment, increasing nitrogen assimilation and/or increase yield and/or increasing the stress tolerance of a plant or plant part comprises introducing into the plant or plant part a heterologous polynucleotide and expressing the heterologous polynucleotide in the plant or plant part. In this method, the expression of the heterologous polynucleotide modulates the level of at least one Dof polypeptide in the plant or plant part, where the Dof polypeptide comprises a Dof domain having an amino acid sequence set forth in SEQ ID NO: 145 or a variant or fragment of the domain.

In specific embodiments, modulation of the level of the Dof polypeptide comprises an increase in the level of at least one Dof polypeptide. In such methods, the heterologous polynucleotide introduced into the plant encodes a polypeptide having a Dof domain or a biologically active variant or fragment thereof. In specific embodiments, the heterologous polynucleotide comprises the sequence set forth in at least one SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153 and/or a biologically active variant or fragment thereof.

In other embodiments, modulating the level of at least one Dof polypeptide comprises decreasing in the level of at least one Dof polypeptide. In such methods, the heterologous polynucleotide introduced into the plant need not encode a functional Dof polypeptide, but rather the expression of the polynucleotide results in the decreased expression of a Dof polypeptide comprising a Dof domain or a biologically active variant or fragment of the Dof domain. In specific embodiments, the Dof polypeptide having the decreased level is set forth in at least one of SEQ ID NOS: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 or a biologically active variant or fragment thereof.

Exemplary promoters that can be used to modulate the level of a Dof polypeptide are described elsewhere herein. In one embodiment, the expression of the heterologous polynucleotide to modulate the level of at least one Dof polypeptide is regulated by a tissue-preferred promoter, particularly, a vascular-preferred promoter, a leaf-preferred promoter (i.e., mesophyll-preferred promoter or a bundle sheath preferred promoter) and/or a seed-preferred promoter (i.e., an endosperm-preferred promoter or an embryo-preferred promoter).

### EXPERIMENTAL

### Example 1

### Sequence Analysis and Expression Data for Maize Dof Sequences

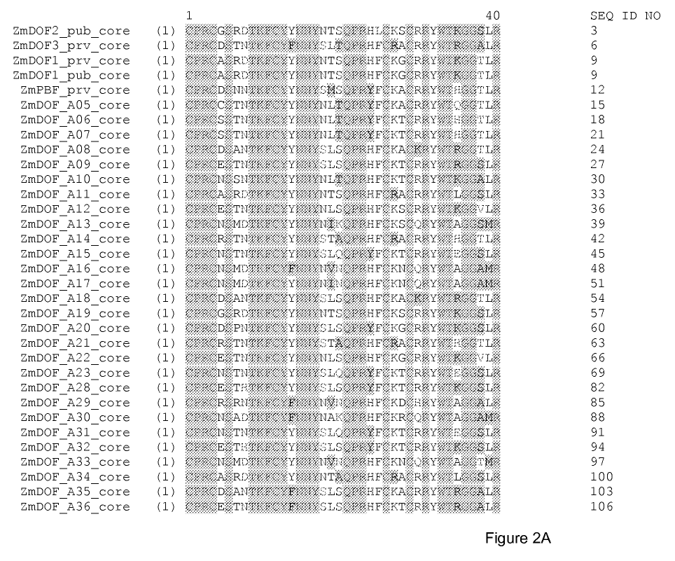

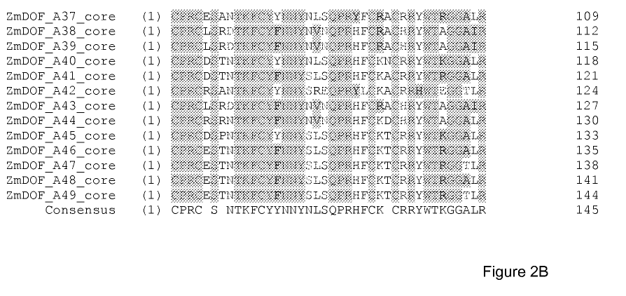

A sequence analysis of the Dof sequences set forth in SEQ ID NOS: 1-144 and 146 was performed. FIG. 2 provides a summary of the Dof domain encoded by SEQ ID NOS: 1-144 and 146. The alignment set forth in FIG. 2 was generated using the “Needle” program in the publicly available EMBOSS suite of tools. This program uses the Needleman-Wunsch algorithm. For proteins, the GAP default parameters (i.e., a gap penalty of 8) were used. See, also, emboss.sourceforge.net/apps/needle.html.

Table 1 provides a summary of the sequences having the highest sequence identity and similarity to the polypeptides encoded by SEQ ID NOS: 1-144 and 146 and 147-160. Table 2 provides a summary of the overall percent sequence identity shared between the polypeptides encoded by SEQ ID NOS: 1-144 and 146. The alignment data provided in Table 2 was generated using the VNT19.0 AlignX tool (Feb. 4, 2002) which is a component of the Vector NTI Suite 7.1.

Table 3 provides a summary of the expression data of the maize Dof sequences and provides the mean parts per million for the indicated tissue with classic MPSS data.

TABLE 1

| | | | | | | Translation |
|---|---|---|---|---|---|---|
| | | | Translation | | Translation | Top Swiss- |
| | | | Top | | Top Swiss- | Prot Hit |
| | | Translation | Swiss-Prot | | Prot Hit Best | Best HSP |
| Assigned | | Top Swiss- | Hit | Translation Top Swiss-Prot | HSP Percent | Percent |
| Gene Name | SEQ ID NO | Prot Hit ID | E-value | Hit Description | Identity | Similarity |
| ZmDOF2_pub | 1, 2, 3 | MNBA_MAIZE | 6.00E−42 | (P38564) DNA-binding protein MNB1A | 46 | 54 |
| ZmDOF3_prv | 4, 5, 6 | MNBA_MAIZE | 3.00E−24 | (P38564) DNA-binding protein MNB1A | 40 | 52 |
| ZmDOF1_prv | 7, 8, 9, 147, | MNBA_MAIZE | 1.00E−139 | (P38564) DNA-binding protein MNB1A | 100 | 100 |
| | 154 | | | | | |
| ZmPBF_prv | 10, 11, 12 | DAG2_ARATH | 2.00E−30 | (Q9ZPY0) DOF zinc finger protein DAG2 (Dof | 33 | 44 |
| | | | | affecting germination 2) | | |
| ZmDOF_A05 | 13, 14, 15, | DAG1_ARATH | 8.00E−29 | (Q43385) DOF zinc finger protein DAG1 (Dof | 35 | 47 |
| | 148, 155 | | | affecting germination 1) (Transcription factor BBFa) | | |
| | | | | (AtBBFa) (rolB domain B factor a) | | |
| ZmDOF_A06 | 16, 17, 18 | DAG2_ARATH | 9.00E−27 | (Q9ZPY0) DOF zinc finger protein DAG2 (Dof | 78 | 87 |
| | | | | affecting germination 2) | | |
| ZmDOF_A07 | 19, 20, 21, | DAG2_ARATH | 9.00E−27 | (Q9ZPY0) DOF zinc finger protein DAG2 (Dof | 78 | 87 |
| | 149, 156 | | | affecting germination 2) | | |
| ZmDOF_A08 | 22, 23, 24 | DAG2_ARATH | 6.00E−26 | (Q9ZPY0) DOF zinc finger protein DAG2 (Dof | 45 | 54 |
| | | | | affecting germination 2) | | |
| ZmDOF_A09 | 25, 26, 37, | DAG2_ARATH | 2.00E−28 | (Q9ZPY0) DOF zinc finger protein DAG2 (Dof | 55 | 64 |
| | 150, 157 | | | affecting germination 2) | | |
| ZmDOF_A10 | 28, 29, 30, | DAG1_ARATH | 7.00E−25 | (Q43385) DOF zinc finger protein DAG1 (Dof | 39 | 60 |
| | 151, 158 | | | affecting germination 1) (Transcription factor BBFa) | | |
| | | | | (AtBBFa) (rolB domain B factor a) | | |
| ZmDOF_A11 | 31, 32, 33 | MNBA_MAIZE | 6.00E−27 | (P38564) DNA-binding protein MNB1A | 59 | 67 |
| ZmDOF_A12 | 34, 35, 36 | MNBA_MAIZE | 1.00E−30 | (P38564) DNA-binding protein MNB1A | 46 | 55 |
| ZmDOF_A13 | 37, 38, 39 | DAG2_ARATH | 7.00E−24 | (Q9ZPY0) DOF zinc finger protein DAG2 (Dof | 58 | 75 |
| | | | | affecting germination 2) | | |
| ZmDOF_A14 | 40, 41, 42, | DAG2_ARATH | 5.00E−25 | (Q9ZPY0) DOF zinc finger protein DAG2 (Dof | 60 | 75 |
| | 152, 159 | | | affecting germination 2) | | |
| ZmDOF_A15 | 43, 44, 45 | DAG2_ARATH | 3.00E−48 | (Q9ZPY0) DOF zinc finger protein DAG2 (Dof | 37 | 48 |
| | | | | affecting germination 2) | | |
| ZmDOF_A16 | 46, 47, 48 | DAG1_ARATH | 2.00E−23 | (Q43385) DOF zinc finger protein DAG1 (Dof | 38 | 58 |
| | | | | affecting germination 1) (Transcription factor BBFa) | | |
| | | | | (AtBBFa) (rolB domain B factor a) | | |
| ZmDOF_A17 | 49, 50, 51 | DAG2_ARATH | 5.00E−24 | (Q9ZPY0) DOF zinc finger protein DAG2 (Dof | 57 | 78 |
| | | | | affecting germination 2) | | |
| ZmDOF_A18 | 52, 53, 54 | DAG2_ARATH | 9.00E−27 | (Q9ZPY0) DOF zinc finger protein DAG2 (Dof | 64 | 73 |
| | | | | affecting germination 2) | | |
| ZmDOF_A19 | 55, 56, 57 | MNBA_MAIZE | 4.00E−53 | (P38564) DNA-binding protein MNB1A | 45 | 54 |
| ZmDOF_A20 | 58, 59, 60 | DAG1_ARATH | 9.00E−26 | (Q43385) DOF zinc finger protein DAG1 (Dof | 68 | 82 |
| | | | | affecting germination 1) (Transcription factor BBFa) | | |
| | | | | (AtBBFa) (rolB domain B factor a) | | |
| ZmDOF_A21 | 61, 62, 63, | DAG2_ARATH | 5.00E−24 | (Q9ZPY0) DOF zinc finger protein DAG2 (Dof | 56 | 70 |
| | 153, 160 | | | affecting germination 2) | | |
| ZmDOF_A22 | 64, 65, 66 | MNBA_MAIZE | 6.00E−28 | (P38564) DNA-binding protein MNB1A | 33 | 46 |
| ZmDOF_A23 | 67, 68, 69 | DAG2_ARATH | 7.00E−35 | (Q9ZPY0) DOF zinc finger protein DAG2 (Dof | 49 | 60 |
| | | | | affecting germination 2) | | |
| ZmDOF_A24 | 70, 71, 72 | DAG2_ARATH | 2.00E−04 | (Q9ZPY0) DOF zinc finger protein DAG2 (Dof | 68 | 79 |
| | | | | affecting germination 2) | | |
| ZmDOF_A25 | 73, 74, 75 | — | — | — | — | — |
| ZmDOF_A26 | 76, 77, 78 | FUS_BOVIN | 2.00E−04 | (Q28009) RNA-binding protein FUS (Pigpen protein) | 36 | 43 |
| ZmDOF_A27 | 79, 80 | MNBA_MAIZE | 3.00E−16 | (P38564) DNA-binding protein MNB1A | 73 | 80 |
| ZmDOF_A28 | 81, 82 | DAG1_ARATH | 5.00E−28 | (Q43385) DOF zinc finger protein DAG1 (Dof | 64 | 77 |
| | | | | affecting germination 1) (Transcription factor BBFa) | | |
| | | | | (AtBBFa) (rolB domain B factor a) | | |
| ZmDOF_A29 | 83, 84, 85 | MNBA_MAIZE | 3.00E−22 | (P38564) DNA-binding protein MNB1A | 56 | 63 |
| ZmDOF_A30 | 86, 87, 88 | DAG2_ARATH | 4.00E−22 | (Q9ZPY0) DOF zinc finger protein DAG2 (Dof | 51 | 72 |
| | | | | affecting germination 2) | | |
| ZmDOF_A31 | 89, 90, 91 | DAG2_ARATH | 3.00E−42 | (Q9ZPY0) DOF zinc finger protein DAG2 (Dof | 34 | 46 |
| | | | | affecting germination 2) | | |
| ZmDOF_A32 | 92, 93, 94 | DOF56_ARATH | 9.00E−36 | Dof zinc finger protein DOF5.6 (AtDOF5.6) | 41 | 50 |
| ZmDOF_A33 | 95, 96, 97 | MNBA_MAIZE | 5.00E−23 | (P38564) DNA-binding protein MNB1A | 39 | 50 |
| ZmDOF_A34 | 98, 99, 100 | MNBA_MAIZE | 3.00E−27 | (P38564) DNA-binding protein MNB1A | 40 | 50 |
| ZmDOF_A35 | 101, 102, 103 | MNBA_MAIZE | 1.00E−26 | (P38564) DNA-binding protein MNB1A | 38 | 47 |
| ZmDOF_A36 | 104, 105, 106 | DAG2_ARATH | 8.00E−27 | (Q9ZPY0) DOF zinc finger protein DAG2 (Dof | 52 | 69 |
| | | | | affecting germination 2) | | |
| ZmDOF_A37 | 107, 108, 109 | MNBA_MAIZE | 1.00E−24 | (P38564) DNA-binding protein MNB1A | 53 | 64 |
| ZmDOF_A38 | 110, 111, 112 | MNBA_MAIZE | 1.00E−22 | (P38564) DNA-binding protein MNB1A | 54 | 59 |
| ZmDOF_A39 | 113, 114, 115 | MNBA_MAIZE | 3.00E−23 | (P38564) DNA-binding protein MNB1A | 54 | 59 |
| ZmDOF_A40 | 116, 117, 118 | DAG2_ARATH | 1.00E−28 | (Q9ZPY0) DOF zinc finger protein DAG2 (Dof | 65 | 82 |
| | | | | affecting germination 2) | | |
| ZmDOF_A41 | 119, 120, 121 | MNBA_MAIZE | 8.00E−27 | (P38564) DNA-binding protein MNB1A | 39 | 48 |
| ZmDOF_A42 | 122, 123, 124 | MNBA_MAIZE | 2.00E−21 | (P38564) DNA-binding protein MNB1A | 46 | 61 |
| ZmDOF_A43 | 125, 126, 127 | MNBA_MAIZE | 2.00E−21 | (P38564) DNA-binding protein MNB1A | 53 | 60 |
| ZmDOF_A44 | 128, 129, 130 | MNBA_MAIZE | 8.00E−22 | (P38564) DNA-binding protein MNB1A | 53 | 63 |
| ZmDOF_A45 | 131, 132, 133 | DAG1_ARATH | 5.00E−29 | (Q43385) DOF zinc finger protein DAG1 (Dof | 52 | 68 |
| | | | | affecting germination 1) (Transcription factor BBFa) | | |
| | | | | (AtBBFa) (rolB domain B factor a) | | |
| ZmDOF_A46 | 134, 135 | DAG2_ARATH | 3.00E−26 | (Q9ZPY0) DOF zinc finger protein DAG2 (Dof | 70 | 85 |
| | | | | affecting germination 2) | | |
| ZmDOF_A47 | 136, 137, 138 | DAG2_ARATH | 8.00E−26 | (Q9ZPY0) DOF zinc finger protein DAG2 (Dof | 53 | 68 |
| | | | | affecting germination 2) | | |
| ZmDOF_A48 | 139, 140, 141 | DAG2_ARATH | 1.00E−25 | (Q9ZPY0) DOF zinc finger protein DAG2 (Dof | 70 | 85 |
| | | | | affecting germination 2) | | |
| ZmDOF_A49 | 142, 143, 144 | DAG2_ARATH | 8.00E−26 | (Q9ZPY0) DOF zinc finger protein DAG2 (Dof | 70 | 85 |
| | | | | affecting germination 2) | | |

TABLE 2

| SEQ ID | 9 | 3 | 56 | 33 | 80 | 39 | 97 | 47 | 88 | 50 | 42 | 63 | 78 | 75 | 100 | 112 | 115 | 127 | 85 | 130 | 109 | 24 | 54 | 124 | 30 |
|---|---|---|---|---|---|---|---|---|---|---|---|---|---|---|---|---|---|---|---|---|---|---|---|---|---|
| 9 | | 47 | 51 | 28 | 72 | 26 | 26 | 25 | 27 | 28 | 29 | 28 | 8 | 5 | 32 | 40 | 30 | 35 | 45 | 31 | 31 | 25 | 25 | 29 | 23 |
| 3 | | | 79 | 27 | 74 | 29 | 27 | 28 | 31 | 29 | 30 | 32 | 7 | 4 | 34 | 46 | 33 | 40 | 53 | 33 | 30 | 30 | 28 | 33 | 23 |
| 56 | | | | 23 | 71 | 22 | 21 | 23 | 27 | 21 | 27 | 28 | 12 | 5 | 24 | 34 | 26 | 33 | 29 | 25 | 32 | 27 | 24 | 26 | 19 |
| 33 | | | | | 75 | 18 | 23 | 21 | 25 | 21 | 31 | 28 | 5 | 0 | 38 | 37 | 25 | 32 | 32 | 28 | 31 | 28 | 29 | 32 | 20 |
| 80 | | | | | | 69 | 71 | 74 | 71 | 69 | 72 | 72 | 0 | 0 | 79 | 81 | 81 | 81 | 78 | 78 | 75 | 67 | 67 | 64 | 69 |
| 39 | | | | | | | 44 | 32 | 32 | 33 | 23 | 26 | 8 | 13 | 20 | 32 | 23 | 30 | 30 | 25 | 25 | 24 | 21 | 24 | 15 |
| 97 | | | | | | | | 32 | 32 | 31 | 24 | 23 | 4 | 5 | 23 | 35 | 27 | 32 | 38 | 30 | 25 | 24 | 22 | 23 | 14 |
| 47 | | | | | | | | | 62 | 48 | 25 | 26 | 4 | 15 | 24 | 40 | 30 | 40 | 36 | 35 | 33 | 24 | 26 | 30 | 17 |
| 88 | | | | | | | | | | 44 | 27 | 29 | 0 | 8 | 27 | 43 | 33 | 40 | 50 | 42 | 33 | 24 | 24 | 32 | 18 |
| 50 | | | | | | | | | | | 26 | 25 | 3 | 7 | 26 | 37 | 29 | 36 | 38 | 31 | 29 | 26 | 25 | 28 | 18 |
| 42 | | | | | | | | | | | | 72 | 0 | 0 | 29 | 36 | 25 | 31 | 38 | 29 | 32 | 32 | 31 | 33 | 26 |
| 63 | | | | | | | | | | | | | 0 | 0 | 29 | 40 | 27 | 32 | 40 | 31 | 36 | 31 | 29 | 33 | 25 |
| 78 | | | | | | | | | | | | | | 30 | 0 | 0 | 0 | 0 | 0 | 0 | 4 | 6 | 7 | 13 | 6 |
| 75 | | | | | | | | | | | | | | | 0 | 0 | 0 | 0 | 0 | 0 | 0 | 17 | 14 | 20 | 9 |
| 100 | | | | | | | | | | | | | | | | 36 | 25 | 34 | 31 | 26 | 34 | 34 | 31 | 32 | 22 |
| 112 | | | | | | | | | | | | | | | | | 94 | 76 | 59 | 51 | 50 | 39 | 40 | 38 | 38 |
| 115 | | | | | | | | | | | | | | | | | | 68 | 58 | 41 | 37 | 28 | 28 | 34 | 26 |
| 127 | | | | | | | | | | | | | | | | | | | 61 | 48 | 45 | 36 | 36 | 34 | 35 |
| 85 | | | | | | | | | | | | | | | | | | | | 83 | 59 | 46 | 47 | 39 | 35 |
| 130 | | | | | | | | | | | | | | | | | | | | | 41 | 33 | 34 | 34 | 27 |
| 109 | | | | | | | | | | | | | | | | | | | | | | 34 | 34 | 37 | 27 |
| 24 | | | | | | | | | | | | | | | | | | | | | | | 77 | 40 | 23 |
| 54 | | | | | | | | | | | | | | | | | | | | | | | | 38 | 22 |
| 124 | | | | | | | | | | | | | | | | | | | | | | | | | 24 |
| 30 | | | | | | | | | | | | | | | | | | | | | | | | | |
| 133 | | | | | | | | | | | | | | | | | | | | | | | | | |
| 60 | | | | | | | | | | | | | | | | | | | | | | | | | |
| 94 | | | | | | | | | | | | | | | | | | | | | | | | | |
| 82 | | | | | | | | | | | | | | | | | | | | | | | | | |
| 45 | | | | | | | | | | | | | | | | | | | | | | | | | |
| 69 | | | | | | | | | | | | | | | | | | | | | | | | | |
| 91 | | | | | | | | | | | | | | | | | | | | | | | | | |
| 27 | | | | | | | | | | | | | | | | | | | | | | | | | |
| 6 | | | | | | | | | | | | | | | | | | | | | | | | | |
| 106 | | | | | | | | | | | | | | | | | | | | | | | | | |
| 135 | | | | | | | | | | | | | | | | | | | | | | | | | |
| 138 | | | | | | | | | | | | | | | | | | | | | | | | | |
| 144 | | | | | | | | | | | | | | | | | | | | | | | | | |
| 141 | | | | | | | | | | | | | | | | | | | | | | | | | |
| 103 | | | | | | | | | | | | | | | | | | | | | | | | | |
| 122 | | | | | | | | | | | | | | | | | | | | | | | | | |
| 36 | | | | | | | | | | | | | | | | | | | | | | | | | |
| 66 | | | | | | | | | | | | | | | | | | | | | | | | | |
| 118 | | | | | | | | | | | | | | | | | | | | | | | | | |
| 12 | | | | | | | | | | | | | | | | | | | | | | | | | |
| 12 | | | | | | | | | | | | | | | | | | | | | | | | | |
| 115 | | | | | | | | | | | | | | | | | | | | | | | | | |
| 18 | | | | | | | | | | | | | | | | | | | | | | | | | |
| 72 | | | | | | | | | | | | | | | | | | | | | | | | | |

| SEQ ID | 133 | 60 | 94 | 82 | 45 | 69 | 91 | 27 | 6 | 106 | 135 | 138 | 144 | 141 | 103 | 122 | 36 | 66 | 118 | 12 | 12 | 115 | 18 | 72 |
|---|---|---|---|---|---|---|---|---|---|---|---|---|---|---|---|---|---|---|---|---|---|---|---|---|
| 9 | 38 | 27 | 27 | 27 | 24 | 37 | 26 | 25 | 27 | 37 | 44 | 24 | 24 | 25 | 27 | 28 | 29 | 30 | 42 | 24 | 24 | 26 | 26 | 19 |
| 3 | 40 | 26 | 25 | 28 | 25 | 43 | 28 | 28 | 24 | 40 | 48 | 27 | 27 | 28 | 31 | 31 | 34 | 34 | 46 | 23 | 23 | 26 | 26 | 18 |
| 56 | 35 | 25 | 23 | 28 | 23 | 36 | 25 | 24 | 24 | 32 | 38 | 25 | 25 | 23 | 26 | 25 | 30 | 31 | 38 | 20 | 20 | 22 | 22 | 17 |
| 33 | 31 | 26 | 23 | 22 | 24 | 39 | 25 | 23 | 24 | 27 | 29 | 24 | 24 | 22 | 23 | 22 | 26 | 28 | 35 | 22 | 22 | 26 | 24 | 17 |
| 80 | 62 | 60 | 64 | 64 | 62 | 62 | 62 | 66 | 73 | 68 | 68 | 68 | 68 | 68 | 69 | 69 | 67 | 64 | 72 | 67 | 67 | 67 | 62 | 75 |
| 39 | 28 | 18 | 22 | 22 | 17 | 35 | 16 | 19 | 16 | 28 | 30 | 17 | 20 | 17 | 19 | 17 | 22 | 24 | 33 | 20 | 20 | 21 | 20 | 13 |
| 97 | 31 | 19 | 22 | 23 | 19 | 32 | 19 | 21 | 20 | 25 | 29 | 18 | 22 | 17 | 19 | 18 | 21 | 23 | 35 | 22 | 22 | 18 | 18 | 12 |
| 47 | 34 | 22 | 25 | 26 | 25 | 36 | 24 | 21 | 23 | 27 | 29 | 22 | 23 | 21 | 22 | 20 | 21 | 24 | 37 | 21 | 21 | 23 | 24 | 15 |
| 88 | 34 | 22 | 25 | 26 | 26 | 38 | 23 | 24 | 23 | 35 | 40 | 25 | 26 | 26 | 24 | 22 | 26 | 28 | 36 | 22 | 22 | 24 | 26 | 18 |
| 50 | 30 | 23 | 23 | 24 | 23 | 36 | 24 | 21 | 22 | 27 | 29 | 22 | 22 | 20 | 24 | 22 | 26 | 30 | 35 | 20 | 20 | 23 | 20 | 13 |
| 42 | 35 | 34 | 25 | 28 | 30 | 39 | 29 | 31 | 37 | 33 | 36 | 31 | 31 | 28 | 32 | 32 | 31 | 37 | 36 | 28 | 28 | 30 | 31 | 26 |
| 63 | 34 | 29 | 24 | 27 | 28 | 41 | 28 | 27 | 36 | 33 | 39 | 30 | 30 | 28 | 31 | 31 | 29 | 33 | 36 | 25 | 25 | 29 | 31 | 25 |
| 78 | 0 | 11 | 6 | 4 | 5 | 0 | 4 | 7 | 7 | 0 | 0 | 6 | 7 | 3 | 6 | 4 | 2 | 5 | 0 | 21 | 21 | 94 | 70 | 67 |
| 75 | 0 | 9 | 9 | 9 | 17 | 0 | 17 | 13 | 5 | 0 | 0 | 5 | 5 | 0 | 0 | 6 | 4 | 5 | 0 | 0 | 0 | 26 | 26 | 26 |
| 100 | 32 | 28 | 28 | 30 | 29 | 37 | 30 | 26 | 31 | 34 | 35 | 30 | 30 | 27 | 27 | 24 | 31 | 34 | 34 | 24 | 24 | 27 | 26 | 18 |
| 112 | 41 | 38 | 40 | 38 | 37 | 36 | 35 | 44 | 50 | 40 | 39 | 40 | 40 | 40 | 44 | 39 | 39 | 42 | 47 | 34 | 34 | 38 | 39 | 36 |
| 115 | 32 | 26 | 28 | 28 | 25 | 31 | 23 | 30 | 31 | 32 | 37 | 27 | 27 | 26 | 33 | 30 | 27 | 28 | 35 | 22 | 22 | 24 | 26 | 18 |
| 127 | 32 | 36 | 31 | 28 | 33 | 34 | 34 | 32 | 39 | 36 | 40 | 34 | 34 | 35 | 37 | 32 | 36 | 38 | 38 | 28 | 28 | 30 | 30 | 22 |
| 85 | 45 | 46 | 39 | 40 | 32 | 39 | 35 | 30 | 53 | 31 | 32 | 37 | 37 | 31 | 34 | 29 | 41 | 46 | 52 | 34 | 34 | 36 | 34 | 52 |
| 130 | 34 | 31 | 32 | 31 | 27 | 33 | 29 | 28 | 34 | 28 | 32 | 30 | 30 | 29 | 28 | 26 | 31 | 35 | 41 | 25 | 25 | 24 | 24 | 18 |
| 109 | 38 | 30 | 26 | 26 | 29 | 46 | 30 | 27 | 31 | 41 | 53 | 28 | 28 | 28 | 31 | 31 | 30 | 31 | 45 | 30 | 30 | 31 | 27 | 17 |
| 24 | 40 | 27 | 27 | 29 | 23 | 43 | 24 | 26 | 26 | 43 | 53 | 27 | 29 | 26 | 29 | 30 | 26 | 31 | 37 | 26 | 26 | 22 | 25 | 17 |
| 54 | 37 | 27 | 24 | 24 | 24 | 41 | 24 | 25 | 24 | 39 | 45 | 23 | 24 | 22 | 29 | 28 | 26 | 32 | 36 | 25 | 25 | 23 | 24 | 17 |
| 124 | 45 | 32 | 30 | 34 | 29 | 46 | 28 | 27 | 33 | 37 | 38 | 27 | 27 | 24 | 29 | 28 | 27 | 26 | 39 | 26 | 26 | 27 | 26 | 19 |
| 30 | 40 | 28 | 25 | 26 | 19 | 34 | 20 | 18 | 22 | 28 | 31 | 22 | 24 | 20 | 22 | 23 | 21 | 24 | 32 | 18 | 18 | 20 | 19 | 11 |
| 133 | | 42 | 37 | 39 | 36 | 40 | 40 | 42 | 38 | 40 | 44 | 41 | 41 | 38 | 40 | 40 | 40 | 48 | 48 | 33 | 33 | 32 | 33 | 21 |
| 60 | | | 29 | 31 | 26 | 45 | 24 | 22 | 24 | 36 | 41 | 25 | 26 | 26 | 27 | 26 | 25 | 29 | 41 | 25 | 25 | 24 | 26 | 16 |
| 94 | | | | 86 | 31 | 45 | 31 | 26 | 26 | 37 | 43 | 26 | 26 | 24 | 30 | 30 | 28 | 29 | 38 | 25 | 25 | 26 | 27 | 18 |
| 82 | | | | | 34 | 46 | 32 | 29 | 30 | 37 | 44 | 28 | 28 | 27 | 32 | 33 | 29 | 33 | 37 | 26 | 26 | 27 | 29 | 18 |
| 45 | | | | | | 87 | 59 | 26 | 24 | 37 | 44 | 26 | 27 | 26 | 27 | 27 | 28 | 27 | 36 | 22 | 22 | 23 | 21 | 13 |
| 69 | | | | | | | 68 | 40 | 40 | 40 | 44 | 42 | 42 | 43 | 40 | 40 | 44 | 43 | 47 | 39 | 39 | 43 | 39 | 32 |
| 91 | | | | | | | | 25 | 24 | 37 | 42 | 26 | 27 | 26 | 29 | 29 | 29 | 27 | 36 | 23 | 23 | 24 | 22 | 13 |
| 27 | | | | | | | | | 34 | 43 | 47 | 36 | 38 | 33 | 32 | 33 | 27 | 29 | 41 | 22 | 22 | 23 | 26 | 20 |
| 6 | | | | | | | | | | 56 | 65 | 39 | 40 | 36 | 37 | 40 | 23 | 27 | 43 | 20 | 20 | 22 | 23 | 15 |
| 106 | | | | | | | | | | | 75 | 88 | 87 | 74 | 50 | 50 | 42 | 43 | 48 | 32 | 32 | 32 | 34 | 32 |
| 135 | | | | | | | | | | | | 84 | 84 | 96 | 54 | 53 | 46 | 47 | 55 | 37 | 37 | 38 | 39 | 42 |
| 138 | | | | | | | | | | | | | 99 | 83 | 44 | 45 | 28 | 28 | 34 | 22 | 22 | 22 | 23 | 17 |
| 144 | | | | | | | | | | | | | | 84 | 43 | 43 | 28 | 28 | 34 | 22 | 22 | 23 | 25 | 18 |
| 141 | | | | | | | | | | | | | | | 43 | 43 | 26 | 26 | 36 | 21 | 21 | 21 | 22 | 16 |
| 103 | | | | | | | | | | | | | | | | 91 | 26 | 28 | 42 | 25 | 25 | 26 | 25 | 20 |
| 122 | | | | | | | | | | | | | | | | | 27 | 29 | 43 | 26 | 26 | 26 | 24 | 19 |
| 36 | | | | | | | | | | | | | | | | | | 76 | 45 | 22 | 22 | 24 | 25 | 17 |
| 66 | | | | | | | | | | | | | | | | | | | 44 | 24 | 24 | 26 | 27 | 18 |
| 118 | | | | | | | | | | | | | | | | | | | | 37 | 37 | 34 | 35 | 23 |
| 12 | | | | | | | | | | | | | | | | | | | | | 100 | 41 | 43 | 36 |
| 12 | | | | | | | | | | | | | | | | | | | | | | 41 | 43 | 36 |
| 115 | | | | | | | | | | | | | | | | | | | | | | | 64 | 59 |
| 18 | | | | | | | | | | | | | | | | | | | | | | | | 95 |
| 72 | | | | | | | | | | | | | | | | | | | | | | | | |

TABLE 3

| | | | | Transcript | | | |
|---|---|---|---|---|---|---|---|
| | | | | maximum | | | |
| | | | 17mer | ppm sample | | | |
| SEQ ID NO | Assigned | | Tag | description | Transcript | Root | |
| for Assigned | Gene | | SEQ ID | (Classic MPSS | maximum | (mean | |
| Gene | Name | MPSS GATC-17mer Tag | NO | Data Only) | ppm | ppm) | Mesocotyl |
| 7, 8, 9 | ZmDOF1_prv | GATCGCGACGACGACCT | 146 | Silk R1 Pollinated 6 h | 1159 | 157.8 | 178.0 |
| 1, 2, 3 | ZmDOF2_pub | GATCTGATTGGGGTGCT | 147 | Stalk VT | 12 | 0.0 | 0.0 |
| 4, 5, 6 | ZmDOF3_prv | GATCATGCACAAATATT | 148 | Embryo R2 | 9 | 0.0 | 0.0 |
| 10, 11, 12 | ZmPBF_prv | GATCATCAGTTCCTATG | 149 | Endosperm R5 | 61 | 0.0 | 0.0 |
| 13, 14, 15 | ZmDOF_A05 | GATCAGCAGCAGCAGCA | 150 | Kernel R3 | 49 | 3.3 | 6.0 |
| 16, 17, 18 | ZmDOF_A06 | GATCGCCGGCAGAGATT | 151 | Endosperm R2 | 288 | 2.9 | 0.0 |
| 19, 20, 21 | ZmDOF_A07 | GATCGCCGGCAGAGATT | 152 | Endosperm R2 | 288 | 2.9 | 0.0 |
| 22, 23, 24 | ZmDOF_A08 | GATCCTCCATTGCCACG | 153 | Root V2 | 51 | 20.2 | 0.0 |
| 25, 26, 27 | ZmDOF_A09 | GATCACACGGTGTCTGG | 154 | Aeriel Vegetative V2 | 35 | 0.7 | 0.0 |
| | | | | Cold, Freezing | | | |
| 28, 29, 30 | ZmDOF_A10 | GATCACTTCAGCCTTCA | 155 | Apical meristem Vn | 451 | 7.3 | 8.0 |
| 34, 35, 36 | ZmDOF_A12 | GATCATTGCGCTGCTGC | 156 | Stalk VT | 156 | 17.8 | 26.0 |
| 37, 38, 39 | ZmDOF_A13 | GATCAGGTTTCCAGCGC | 157 | Aeriel Vegetative V2 | 184 | 15.0 | 47.0 |
| | | | | Cold, Freezing | | | |
| 40, 41, 42 | ZmDOF_A14 | GATCTGTGCAGTGATTT | 158 | Pericarp R3 | 865 | 135.8 | 339.0 |
| 43, 44, 45 | ZmDOF_A15 | GATCGGCGGCAGTGGCG | 159 | Tassel V10-V12 | 86 | 8.9 | 9.0 |
| 46, 47, 48 | ZmDOF_A16 | GATCAATAAGAGGTCCC | 160 | Aeriel Vegetative V2 | 157 | 11.0 | 6.0 |
| | | | | Cold, Freezing | | | |
| 49, 50, 51 | ZmDOF_A17 | GATCGGCGGCGACAAGG | 161 | Aeriel Vegetative Vn | 88 | 3.9 | 0.0 |
| 52, 53, 54 | ZmDOF_A18 | GATCCATTCCACCACCC | 162 | Root V2 | 13 | 1.8 | 0.0 |
| 55, 56, 57 | ZmDOF_A19 | GATCACAAGAACAGCTG | 163 | Stem, Sheath V7-V8 | 37 | 5.3 | 16.0 |
| 58, 59, 60 | ZmDOF_A20 | GATCAACGGGTATACAT | 164 | Silk R1 Pollinated 12 h | 80 | 0.4 | 0.0 |
| 61, 62, 63 | ZmDOF_A21 | GATCTGTGCAGTGATTT | 165 | Pericarp R3 | 865 | 135.8 | 339.0 |
| 64, 65, 66 | ZmDOF_A22 | GATCTTGTGTTTGTTTA | 166 | Aeriel Vegetative Vn | 42 | 0.2 | 13.0 |
| 67, 68, 69 | ZmDOF_A23 | GATCCCTCCGCAGCGTC | 167 | — | na | — | — |
| 70, 71, 72 | ZmDOF_A24 | GATCGCCGGCAGAGATT | 168 | Endosperm R2 | 288 | 2.9 | 0.0 |
| 73, 74, 75 | ZmDOF_A25 | GATCCTTCGCCTTGCTA | 169 | Immature ear V12 | 18 | 0.0 | 0.0 |
| 76, 77, 78 | ZmDOF_A26 | GATCAGCAGCAGCAGCA | 170 | Kernel R3 | 49 | 3.3 | 6.0 |
| 79, 80 | ZmDOF_A27 | GATCGGCCGCCGCCGCC | 171 | Aeriel Vegetative V2 | 5 | 0.0 | 0.0 |
| | | | | Cold, Freezing | | | |
| 81, 82 | ZmDOF_A28 | GATCCTCGCGGGTTCCG | 172 | — | na | — | — |
| 83, 84, 85 | ZmDOF_A29 | GATCATCGCACGACCAA | 173 | Embryo R4 | 7 | 0.0 | 0.0 |
| 86, 87, 88 | ZmDOF_A30 | GATCAATAAGAGGTTCC | 174 | Leaf V6-V8 | 20 | 1.3 | 0.0 |
| 89, 90, 91 | ZmDOF_A31 | GATCTCTCTGGTTGTTT | 175 | Immature ear V19 | 161 | 48.0 | 13.0 |
| 92, 94, 95 | ZmDOF_A32 | GATCCATGTTTGCCGCT | 175 | Immature ear R1 | 51 | 11.4 | 11.0 |
| 95, 96, 97 | ZmDOF_A33 | GATCGATGACCCTGATG | 176 | Embryo R5 | 5 | 0.4 | 0.0 |
| 98, 99, 100 | ZmDOF_A34 | GATCCCTCCGCAACGTC | 177 | — | na | — | — |
| 104, 102, 103 | ZmDOF_A35 | GATCTTGTTTTGGGTCG | 178 | Tassel Spikelet VT | 47 | 2.2 | 0.0 |
| 104, 105, 106 | ZmDOF_A36 | GATCCCGCAGCCGGAGC | 179 | — | na | — | — |
| 107, 108, 109 | ZmDOF_A37 | GATCTGCTGAGGATGTC | 180 | Immature ear V12 | 99 | 4.6 | 11.0 |
| 119, 120, 121 | ZmDOF_A41 | GATCTTGTGTTGGGGTA | 181 | Immature ear Vn | 136 | 3.8 | 32.0 |
| 122, 123, 124 | ZmDOF_A42 | GATCAGCTTGTTTTCTC | 182 | Root V6-V8 | 277 | 42.9 | 13.0 |
| 128, 129, 130 | ZmDOF_A44 | GATCGCAGACCTCTCGG | 183 | Tassel Vn | 20 | 1.2 | 4.0 |
| 131, 132, 133 | ZmDOF_A45 | GATCGAGCTGCTGCAAG | 184 | Tassel Spikelet | 1 | 0.0 | 0.0 |
| 137, 138 | ZmDOF_A46 | GATCCCGCAGCCGGAGC | 185 | — | na | — | — |
| 139, 140, 141 | ZmDOF_A48 | GATCCCGCAGCCGGAGC | 186 | — | na | — | — |

| SEQ ID NO | | | | | | | | | | | |
|---|---|---|---|---|---|---|---|---|---|---|---|
| for Assigned | | | Apical | Immature | | | | | | Tassel | |
| Gene | Leaf | Stalk | Meristem | Ear | Ovary | Embryo | Endosperm | Pericarp | Silk | Spikelet | Pollen |
| 7, 8, 9 | 39.5 | 264.8 | 126.8 | 306.2 | 228.0 | 43.1 | 16.3 | 256.7 | 446.7 | 73.8 | 38.0 |
| 1, 2, 3 | 0.0 | 0.0 | 0.0 | 0.0 | 0.0 | 0.0 | 0.0 | 0.0 | 0.0 | 0.0 | 0.0 |
| 4, 5, 6 | 0.0 | 0.0 | 0.0 | 0.0 | 0.0 | 0.0 | 0.0 | 0.0 | 0.0 | 0.0 | 0.0 |
| 10, 11, 12 | 0.0 | 0.0 | 0.0 | 0.0 | 0.0 | 2.1 | 29.4 | 10.0 | 0.0 | 0.0 | 0.0 |
| 13, 14, 15 | 1.8 | 3.3 | 2.0 | 2.5 | 2.0 | 3.6 | 1.8 | 0.7 | 3.2 | 0.7 | 0.0 |
| 16, 17, 18 | 0.0 | 0.1 | 0.0 | 0.0 | 0.0 | 7.4 | 125.0 | 40.0 | 0.0 | 0.0 | 0.0 |
| 19, 20, 21 | 0.0 | 0.1 | 0.0 | 0.0 | 0.0 | 7.4 | 125.0 | 40.0 | 0.0 | 0.0 | 0.0 |
| 22, 23, 24 | 1.9 | 8.0 | 1.8 | 17.0 | 3.0 | 3.9 | 0.0 | 0.3 | 2.8 | 2.8 | 0.0 |
| 25, 26, 27 | 7.3 | 0.0 | 0.0 | 0.0 | 0.0 | 0.2 | 0.0 | 0.0 | 0.3 | 2.2 | 0.0 |
| 28, 29, 30 | 5.8 | 0.9 | 296.0 | 152.5 | 20.5 | 9.1 | 4.2 | 0.0 | 0.0 | 0.5 | 0.0 |
| 34, 35, 36 | 12.8 | 31.9 | 33.3 | 34.7 | 11.5 | 0.5 | 7.4 | 22.7 | 17.5 | 37.5 | 0.0 |
| 37, 38, 39 | 18.0 | 6.8 | 22.3 | 5.2 | 0.0 | 2.2 | 4.9 | 5.0 | 6.5 | 9.8 | 0.0 |
| 40, 41, 42 | 52.0 | 125.8 | 2.3 | 7.2 | 32.0 | 40.7 | 39.3 | 199.0 | 48.0 | 80.3 | 0.0 |
| 43, 44, 45 | 1.9 | 5.1 | 24.0 | 15.7 | 40.5 | 1.4 | 0.2 | 15.3 | 0.0 | 7.2 | 0.0 |
| 46, 47, 48 | 5.2 | 9.1 | 0.0 | 1.5 | 3.5 | 1.3 | 0.0 | 40.3 | 15.8 | 4.3 | 0.0 |
| 49, 50, 51 | 9.6 | 3.3 | 0.5 | 0.0 | 2.0 | 0.9 | 4.2 | 3.7 | 2.3 | 2.3 | 0.0 |
| 52, 53, 54 | 0.5 | 0.3 | 0.0 | 0.0 | 2.0 | 0.0 | 0.0 | 0.0 | 0.0 | 0.0 | 0.0 |
| 55, 56, 57 | 2.8 | 6.7 | 2.5 | 4.2 | 0.5 | 0.5 | 0.2 | 6.0 | 4.5 | 0.7 | 0.0 |
| 58, 59, 60 | 3.2 | 2.8 | 2.0 | 0.8 | 23.5 | 0.0 | 12.4 | 13.7 | 42.0 | 8.2 | 0.0 |
| 61, 62, 63 | 52.0 | 125.8 | 2.3 | 7.2 | 32.0 | 40.7 | 39.3 | 199.0 | 48.0 | 80.3 | 0.0 |
| 64, 65, 66 | 1.0 | 7.1 | 6.0 | 2.3 | 1.5 | 0.0 | 0.0 | 2.0 | 8.5 | 7.5 | 0.0 |
| 67, 68, 69 | — | — | — | — | — | — | — | — | — | — | — |
| 70, 71, 72 | 0.0 | 0.1 | 0.0 | 0.0 | 0.0 | 7.4 | 125.0 | 40.0 | 0.0 | 0.0 | 0.0 |
| 73, 74, 75 | 0.0 | 0.0 | 0.0 | 0.0 | 0.0 | 0.3 | 0.0 | 0.3 | 0.0 | 0.0 | 0.0 |
| 76, 77, 78 | 1.8 | 3.3 | 2.0 | 2.5 | 2.0 | 3.6 | 1.8 | 0.7 | 3.2 | 0.7 | 0.0 |
| 79, 80 | 0.0 | 0.0 | 0.0 | 0.0 | 0.0 | 0.0 | 0.0 | 0.0 | 0.0 | 0.0 | 0.0 |
| 81, 82 | — | — | — | — | — | — | — | — | — | — | — |
| 83, 84, 85 | 0.0 | 0.0 | 0.0 | 0.0 | 0.0 | 0.0 | 0.0 | 0.0 | 0.0 | 0.0 | 0.0 |
| 86, 87, 88 | 0.1 | 0.0 | 0.0 | 0.0 | 0.0 | 0.0 | 0.0 | 0.0 | 0.0 | 0.2 | 0.0 |
| 89, 90, 91 | 25.5 | 57.9 | 28.8 | 27.0 | 14.5 | 14.9 | 6.0 | 16.0 | 1.7 | 12.8 | 0.0 |
| 92, 94, 95 | 10.5 | 6.3 | 3.8 | 7.0 | 2.0 | 15.0 | 0.9 | 2.0 | 0.0 | 1.2 | 0.0 |
| 95, 96, 97 | 0.1 | 0.0 | 0.0 | 0.0 | 0.0 | 0.5 | 0.3 | 0.0 | 0.0 | 1.2 | 0.0 |
| 98, 99, 100 | — | — | — | — | — | — | — | — | — | — | — |
| 104, 102, 103 | 0.3 | 0.6 | 14.0 | 5.0 | 2.0 | 4.9 | 0.0 | 1.3 | 0.0 | 8.0 | 0.0 |
| 104, 105, 106 | — | — | — | — | — | — | — | — | — | — | — |
| 107, 108, 109 | 0.5 | 1.9 | 17.3 | 7.3 | 9.5 | 4.8 | 1.2 | 0.0 | 1.2 | 0.3 | 0.0 |
| 119, 120, 121 | 18.9 | 3.4 | 65.8 | 64.0 | 26.0 | 9.8 | 0.0 | 4.0 | 1.2 | 11.8 | 0.0 |
| 122, 123, 124 | 3.8 | 8.1 | 1.0 | 0.0 | 2.0 | 0.0 | 1.9 | 14.0 | 12.7 | 3.7 | 0.0 |
| 128, 129, 130 | 0.5 | 3.9 | 0.0 | 1.0 | 8.0 | 0.3 | 0.3 | 0.7 | 0.5 | 7.5 | 0.0 |
| 131, 132, 133 | 0.0 | 0.0 | 0.0 | 0.0 | 0.0 | 0.0 | 0.0 | 0.0 | 0.0 | 0.2 | 0.0 |
| 137, 138 | — | — | — | — | — | — | — | — | — | — | — |
| 139, 140, 141 | — | — | — | — | — | — | — | — | — | — | — |

### Example 2

### Overexpression of Dof Sequences to Modulate Nitrogen Assimilation in Maize

Immature maize embryos from greenhouse donor plants are bombarded with a plasmid containing a Dof sequence (such as Zm-DOF1, 9, 10, 11, 14, 15, 16, 17, 18, 20, 21 or 22) under the control of the UBI promoter and the selectable marker gene PAT (Wohlleben, et al., (1988) Gene 70:25-37), which confers resistance to the herbicide Bialaphos. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the Dof sequence operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂ and 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun (U.S. Pat. No. 5,240,855). All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for an increase in nitrogen use efficiency, increase yield or an increase in stress tolerance.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose and 1.0 ml/l of 0.1 mM abscisic acid (brought to volume with polished D-I H₂O after adjusting to pH 5.6); 3.0 g/l Gelrite (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/l myo-inositol and 40.0 g/l sucrose (brought to volume with polished D-I H₂O after adjusting pH to 5.6) and 6 g/l bacto-agar (added after bringing to volume with polished D-I H₂O), sterilized and cooled to 60° C.

### Example 3

### Suppression of Multiple Dof Sequences in Maize

Multiple maize Dof sequences can be targeted for suppression using an RNAi construct which is designed to target a nucleotide sequence encoding the Dof domain sequence from, for example, Zm-Dof1. Briefly, to target multiple Dof sequences, the DNA sequence encoding the Dof domain (or a sequence having at least 70%, 80%, 90% or greater sequence identity to the Dof domain) is employed and used to make inverted repeats in a vector. For example, a constant having Zm-Dof 1 (Dof Domain):: ADH1 intron 1::ATTB2:: Zm-Dof 1 (Dof Domain can be employed).

For Agrobacterium-mediated transformation of maize with one or more suppression constructs that specifically target at least one Dof sequence, the method of Zhao is employed (U.S. Pat. No. 5,981,840 and PCT Patent Publication Number WO 98/32326, the contents of which are hereby incorporated by reference). Briefly, immature embryos are isolated from maize and the embryos contacted with a suspension of Agrobacterium, where the bacteria are capable of transferring the Dof suppression sequence operably linked to a seed-preferred promoter to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step) and calli grown on selective medium are cultured on solid medium to regenerate the plants.

A decrease of Dof sequence expression can be measured directly by assaying for the level of Dof transcripts or the decrease in expression can be measured by assaying for an increase in nitrogen assimilation, an increase in a stress response or an increase in yield.

### Example 4

### Suppression of Individual Target Dof Sequences in Maize

An individual Dof sequence or, alternatively, a subset of Dof sequences, can be targeted for suppression by using an RNAi construct which is designed to target the desired subset of Dof sequences. For example, DOF1, DOF10 and DOF14 can be individually targeted. Briefly, to suppress individual Dof genes the DNA sequence specific to each Dof (i.e., 3′ UTR, 5′ UTR or specific regions of the CDS) can be used to make the inverted repeats. For example, the Dof 1 3′ UTR can be targeted, a region of the Dof 14 coding sequence or a region of the Dof 10 coding sequence. For example, a construct comprising Zm-Dof 1 (3′ UTR):: ADH1 intron 1:: ATTB2:: Zm-Dof 1 (3′ UTR) or a construct comprising Zm Dof 14:: ADH1 intron 1:: ATTB2:: Zm Dof 14 or a construct comprising Zm Dof 10:: ADH1 intron 1:: ATTB2:: Zm Dof 10 are constructed.

For Agrobacterium-mediated transformation of maize with one or more suppression constructs that specifically target at least one Dof sequence, the method of Zhao is employed (U.S. Pat. No. 5,981,840 and PCT Patent Publication Number WO 98/32326, the contents of which are hereby incorporated by reference). Briefly, immature embryos are isolated from maize and the embryos contacted with a suspension of Agrobacterium, where the bacteria are capable of transferring the Dof suppression sequence operably linked to a seed-preferred promoter to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step) and calli grown on selective medium are cultured on solid medium to regenerate the plants.

A decrease of Dof sequence expression can be measured directly by assaying for the level of Dof transcripts or the decrease in expression can be measured by assaying for an increase in nitrogen assimilation, an increase in a stress response or an increase in yield.

### Example 5

### Modulating Nitrogen Assimilation in Soybean

Soybean embryos are bombarded with a plasmid containing the suppression cassette for at least one Dof sequence operably linked to a leaf-preferred promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050).

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette comprising the suppression cassette for the Dof sequence operably linked to the leaf-preferred promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Manipulation of ZM-DOFs in Maize

The coding regions of several ZM-DOFs were driven by ZM-UBI PRO, a strong constitutive promoter, for overexpression in maize. A terminator sequence (NOS or PINII) was used downstream of the DOFs coding region. In all the transgenic events ‘UBI:MOPAT:PINII’ was used as a herbicide resistant selectable marker and in some of these transgenic ‘ZM-LTP2PRO:RFP:PINII’ was used to sort out the transgenic seeds from the segregating non-transgenic seeds. In some cases other promoter such as ZM-PEPC1 and ZM-GOS2 were also used to drive a mesophyll cell specific and a weak constitutive expression of ZM-DOFs, respectively. All these vectors were transformed in to introEF09B genotype following Agrobacterium-mediated maize transformation protocols. In all the overexpression transgenic events the molecular analysis in terms of transgene copy number, transgene expression and actin control was performed in T0 events. In each construct the T0 events were sorted for high, medium and low transgene expression level within that construct (see, Table 4). Single-copy transgene expressing (and/or RFP expressing seeds) events were selected to advance for further experimentations. ZM-DOF1 allele from B73 inbred when driven by ZM-UBI promoter appeared to be lethal in several different transformation experiments. Hence, the published (Yanagisawa and Izui, (1992) JBC 288:16028) ZM-DOF1 allele from the maize inbred H84 was cloned by RT-PCR. DOF1 allele from B73 and H84 inbreds are 97% identical at protein level. Phosphorylation sites prediction analysis reveal that serine at 47 position, which is a putative phosphorylation site with 100% probability, is present in B73 but missing in H84 allele of ZM-DOF1. Post translational modification (such as phosphorylation) of transcription factors have been known to modify their activities. Transformation experiments with the ZM-DOF1 allele from H84 inbred are currently in progress. RNAi vectors for Zm-DOF1, 7, 10 and 14 were also generated. Two of the DOF1 RNAi vectors (PHP26339 and 26340) were dropped from the list as the molecular analysis of T0 transgenic events didn't show any significant reduction in endogenous DOF1 mRNA. Single transgene copy and transgene expressing plants from 11 different ZM-DOFs related PHPs are currently in the genetic nursery (GN) to bulk up the seeds for further experiments and test crosses for field evaluation in future. Six PHPs are currently in transformation pipe line and T0 events are expected to be in the green house by end of this year. In addition, the Agrobacteria containing 12 different DOF related PHPs are ready for maize transformation. The details of all DOF-related PHPs and their current status are summarized Table 4.

TABLE 4

| | PHP# or | | |
|---|---|---|---|
| EST | pRG# | PROMOTER | GENE |
| cpls1s.pk012.e5 (partial) | PHP25990 | ZM-UBI | ZM-DOF1-B73 |
| cpls1s.pk012.e5 (partial) | PHP25991 | ZM-PEPC1 | ZM-DOF1-B73 |
| cta1n.pk0008.h10 | PHP25995 | ZM-UBI | ZM-DOF14 |
| p0031.ccmay70rb | PHP25996 | ZM-UBI | ZM-DOF17 |
| csc1c.pk006.n23 | PHP25997 | ZM-UBI | ZM-DOF20 |
| p0095.cwsbj71ra | PHP25998 | ZM-UBI | ZM-DOF16 |
| p0111.cipmh48r | PHP26337 | ZM-UBI | ZM-DOF9 |
| cpf1c.pk006.i22a | PHP26338 | ZM-UBI | ZM-DOF10 |
| cpls1s.pk012.e5 (partial) | PHP26339 | ZM-UBI | ZM-DOF1 RNAi |
| cpls1s.pk012.e5 (partial) | PHP26340 | ZM-UBI | ZM-DOF1 RNAi |
| cie1s.pk002.a5 | PHP27070 | ZM-UBI | ZM-DOF10 |
| | | | RNAi |
| cpf1c.pk006.i22a | PHP26910 | ZM-UBI | ZM-DOF11 |
| cen1.pk0109.b4:fis | PHP27748 | ZM-UBI | ZM-DOF7 |
| cen1.pk0109.b4:fis | PHP27749 | ZM-GOS2 | ZM-DOF7 |
| cco1n.pk082.a18 | PHP28364 | ZM-UBI | ZM-DOF12 |
| cds3f.pk001.j20 | PHP28365 | ZM-UBI | ZM-DOF13 |
| cfp5n.pk066.b23 | PHP28866 | ZM-UBI | ZM-DOF5 (7-2) |
| cfp5n.pk066.b23 | PHP28863 | ZM-GOS2 | ZM-DOF5 (7-2) |
| No EST, by RT-PCR | PHP28864 | ZM-UBI | ZM-DOF1-H84 |
| No EST, by RT-PCR | PHP28865 | ZM-PEPC1 | ZM-DOF1-H84 |
| cbn10.pk0039.e7 | PHP26911 | ZM-UBI | ZM-DOF15 |
| cest1s.pk002.f23 | PHP26924 | ZM-UBI | ZM-DOF21 |
| cepe7.pk0012.h2 | PHP26912 | ZM-UBI | ZM-DOF18 |
| cco1n.pk072.j23 | PHP26913 | ZM-UBI | ZM-DOF22 |
| cta1n.pk0008.h10 | PHP26923 | ZM-UBI | ZM-DOF14 |
| | | | RNAi |
| cen1.pk0109.b4:fis | PHP27750 | ZM-UBI | ZM-DOF7 RNAi |
| cpls1s.pk012.e5 (partial) | pRG974 | 35S-TET-OP | ZM-DOF1-B73 |
| cfp7n.pk005.a14 | pRG1020 | ZM-UBI | ZM-DOF8 |
| cfp3n.pk069.i19 | pRG1029 | ZM-UBI | ZM-DOF28 |
| cfp7n.pk069.o21 | pRG1021 | ZM-UBI | ZM-DOF30 |
| cfp1n.pk071.e24 | pRG1022 | ZM-UBI | ZM-DOF33 |
| cfp2n.pk002.k24 | pRG1023 | ZM-UBI | ZM-DOF34 |

Continued detailed phenotypic analysis (visual, molecular, biochemical/enzymatic, physiological, stress etc.) of these maize transgenic events is ongoing.

### Example 7

### Overexpression of ZM-DOFs in Arabidopsis

In addition to overexpressing ZM-DOFs in maize, Arabidopsis transgenic lines overexpressing ZM-DOFs under the control a constitutive ZM-UBI promoter were generated. In some cases a week constitutive (ZM-GOS2) or a mysophill cell specific promoter (ZM-PEPC1) was also used to drive the expression of ZM-DOFs. A terminator sequence (NOS or PINII) was used downstream of the DOFs coding region. In all the transgenic events ‘UBI:MOPAT:PINII’ was used as a herbicide resistant selectable marker. These overexpression vectors were transformed in to Arabidopsis thaliana ecotype Columbia-0 by Agrobacterium mediated ‘Floral-Dip’ method (Clough and Bent, (1998) Plant Journal 16:735). T0 seeds were screened for T1 transformants in soil for herbicide resistance. For molecular analysis of the transgenic T1 events, RT-PCRs were conducted to detect the transgene expression, actin control and the presence of genomic DNA in the RNA preparations. In each construct the T1 events were sorted for high, medium and low transgene expression level within that construct (see attached, Table 5). Transgene expressing events were advanced for further studies. In total Arabidopsis transgenic lines were generated for 26 different overexpression PHPs representing 22 different ZM-DOFs. The status of various ZM-DOFs overexpression experiments in Arabidopsis is summarized in the following table.

TABLE 5

| EST | PHP#/pRG# | PROMOTER | GENE |
|---|---|---|---|
| RT-PCR | PHP25990 | ZM-UBI | ZM-DOF1-B73 |
| RT-PCR | PHP25991 | ZM-PEPC1 | ZM-DOF1-B73 |
| No EST, by RT-PCR | PHP28864 | ZM-UBI | ZM-DOF1-H84 |
| No EST, by RT-PCR | PHP28865 | ZM-PEPC1 | ZM-DOF1-H84 |
| cfp5n.pk066.b23 | PHP28863 | ZM-GOS2 | ZM-DOF5 |
| cfp5n.pk066.b23 | PHP28866 | ZM-UBI | ZM-DOF5 |
| cen1.pk0109.b4:fis | PHP27748 | ZM-UBI | ZM-DOF7 |
| cen1.pk0109.b4:fis | PHP27749 | ZM-GOS2 | ZM-DOF7 |
| cfp7n.pk005.a14 | pRG1020 | ZM-UBI | ZM-DOF8 |
| p0111.cipmh48r | PHP26337 | ZM-UBI | ZM-DOF9 |
| cpf1c.pk006.i22a | PHP26338 | ZM-UBI | ZM-DOF10 |
| cpf1c.pk006.i22a | PHP26910 | ZM-UBI | ZM-DOF11 |
| cco1n.pk082.a18 | PHP28364 | ZM-UBI | ZM-DOF12 |
| cds3f.pk001.j20 | PHP28365 | ZM-UBI | ZM-DOF13 |
| cta1n.pk0008.h10 | PHP25995 | ZM-UBI | ZM-DOF14 |
| cbn10.pk0039.e7 | PHP26911 | ZM-UBI | ZM-DOF15 |
| p0095.cwsbj71ra | PHP25998 | ZM-UBI | ZM-DOF16 |
| p0031.ccmay70rb | PHP25996 | ZM-UBI | ZM-DOF17 |
| cepe7.pk0012.h2 | PHP26912 | ZM-UBI | ZM-DOF18 |
| csc1c.pk006.n23 | PHP25997 | ZM-UBI | ZM-DOF20 |
| cest1s.pk002.f23 | PHP26924 | ZM-UBI | ZM-DOF21 |
| cco1n.pk072.j23 | PHP26913 | ZM-UBI | ZM-DOF22 |
| cfp3n.pk069.i19 | pRG1029 | ZM-UBI | ZM-DOF28 |
| cfp7n.pk069.o21 | pRG1021 | ZM-UBI | ZM-DOF30 |
| cfp1n.pk071.e24 | pRG1022 | ZM-UBI | ZM-DOF33 |
| cfp2n.pk002.k24 | pRG1023 | ZM-UBI | ZM-DOF34 |

Continued detailed phenotypic analysis (visual, molecular, biochemical/enzymatic, physiological, stress etc.) of these Arabidopsis transgenic lines is ongoing.

### Example 8

### UBI PRO::ZM-DOF1-H84 (PHP28865) Overexpression in Arabidopsis Up-Regulates AtPEPC1 and AtPPDK1

In an initial experiment to overexpress in Arabidopsis the ZM-DOF1 allele from B73 maize inbred under the control of ZM-UBI promoter did not yield any transgene expressing events. This observation was consistent with that found in maize transformation experiments. A clone of the published ZM-DOF1 (Yanagisawa and Izui, (1992) JBC 288:16028) allele from H84 maize inbred by RT-PCR on young leaf RNA was prepared. DOF1 allele from B73 and H84 inbreds are 97% identical at protein level. Over-expression of ZM-DOF1 allele from H84 inbred with ZM-UBI promoter resulted in several transgene expressing events and several of the events showed a significant up-regulation of AtPEPC1 (At3g14940) and AtPPDK1 (At5g08570), which is consistent with the results reported in a published study (Yanagisawa, et al., (2004) PNAS 101:7833). Experiments also generated several transgenic Arabidopsis lines overexpressing ZM-DOF1 alleles from both B73 and H84 inbreds under the control of a mesopyhl cell specific ZM-PEPC1 promoter having no detectable significant difference in AtPEPC1, AtPPDK1 expression levels.

### Example 9

### ZM-DOF7 is an Endosperm Specific Gene

Initial Lynx MPSS expression analysis showed that ZM-DOF7 is expressed only in endosperm libraries. In order to confirm this observation, a saturated RT-PCRs (35 cycles) were conducted on total RNA isolated from various organs of maize inbred B73. Total RNAs isolated from V3 seedlings, immature ear, mature leaf, endosperm (14DAP), young leaf, internode and roots were used to perform RT-PCR experiment with ZM-DOF7 gene specific (P894BC#11843 and P895BC#188944) and AT-Actin2 (P815BC#99547 and P816BC#99548) primers. The RT-PCR data reveals that ZM-DOF7 is expressed in endosperm (14DAP) only among different organs tested.

### Example 10

### Sub-Cellular Localization of ZM-DOF10 and ZM-DOF14

In order to determine the sub-cellular localization, ZM-DOF10 and ZM-DOF14 were tagged with RFP and driven by a strong constitutive ZM-UBI promoter. A vector expressing RFP alone under the control of ZM-UBI promoter was also used as a control. These three constructs were bombarded into the onion epidermal cells for RFP fusion protein localization. The results clearly indicate the ZM-DOF10-RFP and ZM-DOF14-RFP are predominantly localized in nucleus whereas RFP alone is present more or less everywhere (e.g., cytosol). Initial Lynx MPSS expression analysis showed that ZM-DOF10 is expressed in apical meristems and immature ear libraries. To further determine the spatial expression pattern of ZM-DOF10, in-situ hybridization experiments were performed on V3 shoot apical meristem (SAM) and immature ear tips and bases. The comparison of the data from hybridization of sense and antisense strands of ZM-DOF10 suggests that DOF10 was expressed in specific cell layers in V3 SAM whereas in immature ear this gene is expressed only in the tip (actively growing cells, meristem).

### Example 11

### Variants of Dof Sequences

A. Variant Nucleotide Sequences of Dof Sequences That Do Not Alter the Encoded Amino Acid Sequence

The Dof nucleotide sequence set forth in SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153 is used to generate variant nucleotide sequences having the nucleotide sequence of the open reading frame with about 70%, 76%, 81%, 86%, 92% and 97% nucleotide sequence identity when compared to the starting unaltered ORF nucleotide sequence of the appropriate SEQ ID NO. These functional variants are generated using a standard codon table. While the nucleotide sequence of the variant is altered, the amino acid sequence encoded by the open reading frame does not change.

B. Variant Amino Acid Sequences of a Dof Sequence

Variant amino acid sequences of Dof sequence are generated. In this example, one amino acid is altered. Specifically, the open reading frame set forth in SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 is reviewed to determined the appropriate amino acid alteration. The selection of the amino acid to change is made by consulting the protein alignment (with the other orthologs and other gene family members from various species). See, FIG. 1 and Table 1. An amino acid is selected that is deemed not to be under high selection pressure (not highly conserved) and which is rather easily substituted by an amino acid with similar chemical characteristics (i.e., similar functional side-chain). Using the protein alignment set forth in FIG. 1, Table 1 and the consensus sequence set forth in SEQ ID NO: 145, an appropriate amino acid can be changed. Once the targeted amino acid is identified, the procedure outlined in Example 6A is followed. Variants having about 70%, 75%, 81%, 86%, 92% and 97% nucleic acid sequence identity to SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160 are generated using this method.

C. Additional Variant Amino Acid Sequences of Dof Sequences

In this example, artificial protein sequences are created having 82%, 87%, 92% and 97% identity relative to the reference protein sequence. This latter effort requires identifying conserved and variable regions from the alignment set forth in FIGS. 1 and 2 and then the judicious application of an amino acid substitutions table. These parts will be discussed in more detail below.

Largely, the determination of which amino acid sequences are altered is made based on the conserved regions among Dof protein or among the other Dof proteins. See, FIGS. 1, 2 and Table 1. Based on the sequence alignment, the various regions of the Dof sequences that can likely be altered are represented in lower case letters, while the conserved regions are represented by capital letters. It is recognized that conservative substitutions can be made in the conserved regions below without altering function. In addition, one of skill will understand that functional variants of the Dof sequence of the invention can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 6.

TABLE 6
Substitution Table

| | | Rank of | |
|---|---|---|---|
| | | Order | |
| Amino | Strongly Similar and | to | |
| Acid | Optimal Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involved a 50:50 substitution of the two alternate optimal substitutions.

The variant amino acid sequences are written as output. Perl script is used to calculate the percent identities. Using this procedure, variants of Dof sequences are generating having about 82%, 87%, 92% and 97% amino acid identity to the starting unaltered ORF nucleotide sequence of the corresponding SEQ ID NO.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

That which is claimed:

1. An isolated polypeptide comprising an amino acid sequence selected from the group consisting of:
(a) the amino acid sequence comprising SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160;
(b) the amino acid sequence comprising at least 90% sequence identity to SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 167, 158, 159 or 160 wherein said polypeptide has the ability to modulate transcription; and,
(c) the amino acid sequence comprising at least 40 consecutive amino acids of SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160, wherein said polypeptide retains the ability to modulate transcription.

2. An isolated polynucleotide comprising a nucleotide sequence selected from the group consisting of:
(a) the nucleotide sequence comprising SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153;
(b) the nucleotide sequence encoding an amino acid sequence comprising SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144, 154, 155, 156, 157, 158, 159 or 160;
(c) the nucleotide sequence comprising at least 90% sequence identity to SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153 wherein said polynucleotide encodes a polypeptide having the ability to modulate transcription or the expression of the polynucleotide in a plant decrease the expression of at least one Dof polypeptide;
(d) the nucleotide sequence comprising at least 40 consecutive nucleotides of SEQ ID NO: 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 136, 137, 139, 140, 142, 143, 146, 147, 148, 149, 150, 151, 152 or 153 or a complement thereof, wherein said polynucleotide encodes a polypeptide having the ability to modulate transcription or the expression of the polynucleotide in a plant decrease the expression of at least one Dof polypeptide;
(e) the nucleotide sequence that hybridizes under stringent conditions to the complement of the nucleotide sequence of a) or b), wherein said stringent conditions comprise hybridization in 50% formamide, 1 M NaCl, 1% SDS at 37° C. and a wash in 0.1×SSC at 60° C. to 65° C., wherein said polynucleotide encodes a polypeptide having the ability to modulate transcription or the expression of the polynucleotide in a plant decrease the expression of at least one Dof polypeptide; and,
(f) the nucleotide sequence encoding an amino acid sequence having at least 90% sequence identity to SEQ ID NO: 3, 6, 9, 12, 15, 18, 21, 24, 27, 30, 33, 36, 39, 42, 45, 48, 51, 54, 57, 60, 63, 66, 69, 72, 75, 78, 80, 82, 85, 88, 91, 94, 97, 100, 103, 106, 109, 112, 115, 118, 121, 124, 127, 130, 133, 135, 138, 141, 144. 154, 155, 156, 157, 158, 159 or 160 wherein said polynucleotide encodes a polypeptide having has the ability to modulate transcription or expression of the polynucleotide in a plant decrease the expression of at least one Dof polypeptide.

3. An expression cassette comprising the polynucleotide of claim 2.

4. The expression cassette of claim 3, wherein said polynucleotide is operably linked to a promoter that drives expression in a plant.

5. The expression cassette of claim 4, wherein said polynucleotide is operably linked to a tissue-preferred promoter, a constitutive promoter or an inducible promoter.

6. The expression cassette of claim 5, wherein said tissue-preferred promoter is a leaf-preferred promoter, a mesophyll-preferred promoter, a bundle sheath-preferred promoter, a seed-preferred promoter, an endosperm-preferred promoter or an embryo-preferred promoter.

7. A plant or plant part comprising a polynucleotide operably linked to a promoter that drives expression in the plant, wherein said polynucleotide comprises the nucleotide sequence of claim 2.

8. The plant or plant part of claim 7, wherein said plant is a monocot.

9. The plant or plant part of claim 8, wherein said monocot is maize, wheat, rice, barley, sorghum or rye.

10. The plant or plant part of claim 8, wherein said monocot is maize.

11. The plant or plant part of claim 7, wherein said plant is a dicot.

12. The plant or plant part of claim 11, wherein the dicot is soybean, Brassica, sunflower, cotton or alfalfa.

13. The plant or plant part of any one of claims 7 to 12, wherein said polynucleotide is stably incorporated into the genome of the plant.

14. The plant part of any one of claims 7 to 13, wherein said plant part is a cell.

15. A seed having stably incorporated into its genome the polynucleotide of claim 2.

16. A method for modulating the level of a Dof polypeptide in a plant or a plant part comprising introducing into said plant or plant part a heterologous polynucleotide comprising a nucleotide sequence of claim 2 and expressing said heterologous polynucleotide.

17. The method of claim 16, wherein said polynucleotide is stably integrated into the genome of the plant or plant part.

18. The method of claim 16 or 17, wherein said plant is a dicot.

19. The method of claim 18, wherein said dicot is soybean, Brassica, sunflower, cotton or alfalfa.

20. The method of claim 16 or 17, wherein said plant is a monocot.

21. The method of claim 20, wherein said monocot is maize, wheat, rice, barley, sorghum or rye.

22. The method of any one of claims 16-21, wherein said polynucleotide is operably linked to a tissue-preferred promoter, a constitutive promoter or an inducible promoter.

23. The method of claim 22, wherein said tissue-preferred promoter is a leaf-preferred promoter, a mesophyll-preferred promoter, a bundle sheath-preferred promoter, a seed-preferred promoter, an endosperm-preferred promoter or an embryo-preferred promoter.

24. The method of any one of claims 16-23, wherein the level of the Dof polypeptide is increased.

25. The method of any one of claims 16-23, wherein the level of the Dof polypeptide is decreased.

26. The method of any one of claims 16-25, wherein the yield of the plant is increased.

27. The method of any one of claims 16-25, wherein the nitrogen use efficiency in said plant or plant part is increased.

28. The method of any one of claims 16-25 wherein the stress response of the plant is improved.

29. A method for increasing nitrogen use efficiency in a plant comprising
a) introducing into said plant a heterologous polynucleotide; and,
b) expressing said polynucleotide in the plant from an operably linked leaf-preferred promoter or a vascular preferred promoter;
wherein expression of said heterologous polynucleotide modulates the level of at least one Dof polypeptide comprising an amino acid sequence set forth in SEQ ID NO: 148 or a biologically active variant or fragment thereof, wherein said biologically active variant comprises at least 80% sequence identity to SEQ ID NO: 145 and said Dof polypeptide is capable of modulating transcription.

30. A method for increasing yield in a plant comprising
a) introducing into said plant a heterologous polynucleotide; and,
b) expressing said polynucleotide in the plant from an operably linked leaf-preferred promoter or a vascular preferred promoter;
wherein expression of said heterologous polynucleotide modulates the level of at least one Dof polypeptide comprising an amino acid sequence set forth in SEQ ID NO: 145 or a biologically active variant or fragment thereof, wherein said biologically active variant comprises at least 80% sequence identity to SEQ ID NO: 145 and said Dof polypeptide is capable of modulating transcription.

31. A method for improving the stress response of a plant comprising
a) introducing into said plant a heterologous polynucleotide; and,
b) expressing said polynucleotide in the plant from an operably linked leaf-preferred promoter or a vascular preferred promoter;
wherein expression of said heterologous polynucleotide modulates the level of at least one Dof polypeptide comprising an amino acid sequence set forth in SEQ ID NO: 145 or a biologically active variant or fragment thereof, wherein said biologically active variant comprises at least 80% sequence identity to SEQ ID NO: 145 and said Dof polypeptide is capable of modulating transcription.

32. The method of claim 29, 30 or 31, wherein said heterologous polynucleotide encodes a Dof polypeptide.

33. The method of claim 29, 30 or 31, wherein expression of said heterologous polynucleotide decreases the level of at least one Dof polynucleotide.

34. The method of claim 29, 30, 31, 32 or 33, wherein said leaf-preferred promoter comprises a bundle sheath-preferred promoter or a mesophyll-preferred promoter.

35. An isolated expression cassette comprising a polynucleotide operably linked to a heterologous leaf-preferred promoter or a vascular preferred promoter, wherein said polynucleotide is selected from the group consisting of:
a) a polynucleotide encoding a Dof polypeptide comprising an amino acid sequence set forth in SEQ ID NO: 145;
b) a polynucleotide encoding a Dof polypeptide comprising an amino acid sequence having at least 80% sequence identity to SEQ ID NO: 145, said Dof polypeptide is capable of modulating transcription; and,
c) a polynucleotide which when expressed in a plant decrease the expression level of a Dof polypeptide comprising an amino acid sequence set forth in SEQ ID NO: 145; and,
d) a polynucleotide which when expressed in a plant decrease the expression level of a Dof polypeptide comprising an amino acid sequence having at least 80% sequence identity to SEQ ID NO: 145, said Dof polypeptide is capable of modulating transcription.

36. A plant or plant part comprising a heterologous expression cassette of claim 35.

37. The plant or plant part of claim 36, wherein said plant is a monocot.

38. The plant or plant part of claim 37, wherein said monocot is maize, wheat, rice, barley, sorghum or rye.

39. The plant or plant part of claim 36, wherein said plant is a dicot.

40. The plant or plant part of claim 39, wherein the dicot is soybean, Brassica, sunflower, cotton or alfalfa.

41. The plant or plant part of any one of claims 35 to 40, wherein said polynucleotide is stably incorporated into the genome of the plant.

42. The plant part of any one of claims 35 to 41, wherein said plant part is a cell.

43. A seed having stably incorporated into its genome the expression cassette of claim 35.
